SEQUENCE LISTING (1406 sequences)

SEQ ID NO: 1 - protein, 945 aa, Artificial Sequence
  Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 2 - protein, 315 aa, Artificial Sequence
  Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 3-493 - skipped (491 entries)

SEQ ID NO: 494 - protein, 5 aa, Unknown
  Synthetic peptide
  MISC_FEATURE: Silk polypeptide block sequence
SGAGG

SEQ ID NO: 495 - protein, 5 aa, Unknown
  Synthetic peptide
  MISC_FEATURE: Silk polypeptide block sequence
GSGAG

SEQ ID NO: 496 - protein, 5 aa, Unknown
  Synthetic peptide
  MISC_FEATURE: Silk polypeptide block sequence
GGSGA

SEQ ID NO: 497-1114 - skipped (618 entries)

SEQ ID NO: 1115 - protein, 174 aa, Deinopis spinosa
GAGYGAGAGAGGGAGAGTGYGGGAGYGTGSGAGYGAGVGYGAGAGAGGGAGAGAGGGTGAGAGGGAGAGYGAGTGYGAGAGAGGGAGAGAGAGAGAGAGAGSGAGAGYGAGAGYGAGAGAGGVAGAGAAGGAGAAGGAGAAGGAGAAGGAGAGAGAGSGAGAGAGGGARAGAGG

SEQ ID NO: 1116-1225 - skipped (110 entries)

SEQ ID NO: 1226 - protein, 149 aa, Latrodectus hesperus
GGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGA

SEQ ID NO: 1227-1233 - skipped (7 entries)

SEQ ID NO: 1234 - protein, 161 aa, Trichonephila clavipes
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 1235-1238 - skipped (4 entries)

SEQ ID NO: 1239 - protein, 186 aa, Nephilingis cruentata
GAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAA

SEQ ID NO: 1240-1245 - skipped (6 entries)

SEQ ID NO: 1246 - protein, 132 aa, Uloborus diversus
GSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAA

SEQ ID NO: 1247-1248 - skipped (2 entries)

SEQ ID NO: 1249 - protein, 198 aa, Uloborus diversus
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 1250-1311 - skipped (62 entries)

SEQ ID NO: 1312 - protein, 190 aa, Araneus ventricosus
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 1313-1344 - skipped (32 entries)

SEQ ID NO: 1345 - protein, 166 aa, Dolomedes tenebrosus
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 1346-1381 - skipped (36 entries)

SEQ ID NO: 1382 - protein, 177 aa, Nephilingis cruentata
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 1383 - protein, 174 aa, Nephilingis cruentata
GGAGQGGYGGLGGQGAGAAAAAAGGAGQGGYGGQGAGQGAAAAAASGAGQGGYGGPGAGQGAGAAAAAAGGAGQGGYGGLGGQGAGQGAGAAAAAAGGAGQGGYGGQGAGQGAAAAAAGGAGQGGYGGLGSGQGGYGGQGAGAAAAAGGAGQGGYGGLGGQGAGQGAGAAAAAA

SEQ ID NO: 1384 - protein, 181 aa, Aliatypus gulosus
GAASSSSTIITTKSASASAAADASAAATASAASRSSANAAASAFAQSFSSILLESGYFCSIFGSSISSSYAAAIASAASRAAAESNGYTTHAYACAKAVASAVERVTSGADAYAYAQAISDALSHALLYTGRLNTANANSLASAFAYAFANAAAQASASSASAGAASASGAASASGAGSAS

SEQ ID NO: 1385 - protein, 126 aa, Plectreurys tristis
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 1386 - protein, 239 aa, Plectreurys tristis
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 1387 - protein, 182 aa, Araneus gemmoides
GNVGYQLGLKVANSLGLGNAQALASSLSQAVSAVGVGASSNAYANAVSNAVGQVLAGQGILNAANAGSLASSFASALSSSAASVASQSASQSQAASQSQAAASAFRQAASQSASQSDSRAGSQSSTKTTSTSTSGSQADSRSASSSASQASASAFAQQSSASLSSSSSFSSAFSSATSISAV

SEQ ID NO: 1388 - protein, 180 aa, Argiope aurantia
GSLASSFASALSASAASVASSAAAQAASQSQAAASAFSRAASQSASQSAARSGAQSISTTTTTSTAGSQAASQSASSAASQASASSFARASSASLAASSSFSSAFSSANSLSALGNVGYQLGFNVANNLGIGNAAGLGNALSQAVSSVGVGASSSTYANAVSNAVGQFLAGQGILNAANA

SEQ ID NO: 1389 - protein, 199 aa, Deinopis spinosa
GASASAYASAISNAVGPYLYGLGLFNQANAASFASSFASAVSSAVASASASAASSAYAQSAAAQAQAASSAFSQAAAQSAAAASAGASAGAGASAGAGAVAGAGAVAGAGAVAGASAAAASQAAASSSASAVASAFAQSASYALASSSAFANAFASATSAGYLGSLAYQLGLTTAYNLGLSNAQAFASTLSQAVTGVGL

SEQ ID NO: 1390 - protein, 171 aa, Trichonephila clavipes
GATAASYGNALSTAAAQFFATAGLLNAGNASALASSFARAFSASAESQSFAQSQAFQQASAFQQAASRSASQSAAEAGSTSSSTTTTTSAARSQAASQSASSSYSSAFAQAASSSLATSSALSRAFSSVSSASAASSLAYSIGLSAARSLGIADAAGLAGVLARAAGALGQ

SEQ ID NO: 1391 - protein, 268 aa, Argiope trifasciata
GGAPGGGPGGAGPGGAGFGPGGGAGFGPGGGAGFGPGGAAGGPGGPGGPGGPGGAGGYGPGGAGGYGPGGVGPGGAGGYGPGGAGGYGPGGSGPGGAGPGGAGGEGPVTVDVDVTVGPEGVGGGPGGAGPGGAGFGPGGGAGFGPGGAPGAPGGPGGPGGPGGPGGPGGVGPGGAGGYGPGGAGGVGPAGTGGFGPGGAGGFGPGGAGGFGPGGAGGFGPAGAGGYGPGGVGPGGAGGFGPGGVGPGGSGPGGAGGEGPVTVDVDVSV

SEQ ID NO: 1392 - protein, 420 aa, Trichonephila clavipes
GVSYGPGGAGGPYGPGGPYGPGGEGPGGAGGPYGPGGVGPGGSGPGGYGPGGAGPGGYGPGGSGPGGYGPGGSGPGGYGPGGSGPGGYGPGGSGPGGYGPGGYGPGGSGPGGSGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAAPGGAGPGGAGPGGAGPGGAGPGGAGPGGAGPGGAGGAGGAGGSGGAGGSGGTTIIEDLDITIDGADGPITISEELPISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGP

SEQ ID NO: 1393 - protein, 376 aa, Latrodectus hesperus
GINVDSDIGSVTSLILSGSTLQMTIPAGGDDLSGGYPGGFPAGAQPSGGAPVDFGGPSAGGDVAAKLARSLASTLASSGVFRAAFNSRVSTPVAVQLTDALVQKIASNLGLDYATASKLRKASQAVSKVRMGSDTNAYALAISSALAEVLSSSGKVADANINQIAPQLASGIVLGVSTTAPQFGVDLSSINVNLDISNVARNMQASIQGGPAPITAEGPDFGAGYPGGAPTDLSGLDMGAPSDGSRGGDATAKLLQALVPALLKSDVFRAIYKRGTRKQVVQYVTNSALQQAASSLGLDASTISQLQTKATQALSSVSADSDSTAYAKAFGLAIAQVLGTSGQVNDANVNQIGAKLATGILRGSSAVAPRLGIDLS

SEQ ID NO: 1394 - protein, 200 aa, Argiope trifasciata
GAGYTGPSGPSTGPSGYPGPLGGGAPFGQSGFGGSAGPQGGFGATGGASAGLISRVANALANTSTLRTVLRTGVSQQIASSVVQRAAQSLASTLGVDGNNLARFAVQAVSRLPAGSDTSAYAQAFSSALFNAGVLNASNIDTLGSRVLSALLNGVSSAAQGLGINVDSGSVQSDISSSSSFLSTSSSSASYSQASASSTS

SEQ ID NO: 1395 - protein, 357 aa, Uloborus diversus
GASAADIATAIAASVATSLQSNGVLTASNVSQLSNQLASYVSSGLSSTASSLGIQLGASLGAGFGASAGLSASTDISSSVEATSASTLSSSASSTSVVSSINAQLVPALAQTAVLNAAFSNINTQNAIRIAELLTQQVGRQYGLSGSDVATASSQIRSALYSVQQGSASSAYVSAIVGPLITALSSRGVVNASNSSQIASSLATAILQFTANVAPQFGISIPTSAVQSDLSTISQSLTAISSQTSSSVDSSTSAFGGISGPSGPSPYGPQPSGPTFGPGPSLSGLTGFTATFASSFKSTLASSTQFQLIAQSNLDVQTRSSLISKVLINALSSLGISASVASSIAASSSQSLLSVSA

SEQ ID NO: 1396 - protein, 32 aa, Euprosthenops australis
GGQGGQGQGRYGQGAGSSAAAAAAAAAAAAAA

SEQ ID NO: 1397 - protein, 42 aa, Tetragnatha kauaiensis
GGLGGGQGAGQGGQQGAGQGGYGSGLGGAGQGASAAAAAAAA

SEQ ID NO: 1398 - protein, 42 aa, Argiope aurantia
GGYGPGAGQQGPGSQGPGSGGQQGPGGLGPYGPSAAAAAAAA

SEQ ID NO: 1399 - protein, 46 aa, Deinopis spinosa
GPGGYGGPGQQGPGQGQYGPGTGQQGQGPSGQQGPAGAAAAAAAAA

SEQ ID NO: 1400 - protein, 42 aa, Trichonephila clavata
GPGGYGLGQQGPGQQGPGQQGPAGYGPSGLSGPGGAAAAAAA

SEQ ID NO: 1401 - protein, 1600 aa, Artificial Sequence
  Synthetic polypeptide
  SITE (1)..(1600): This sequence may encompass 2-20 “GGY-[GPG-X1]n1-GPS-(A)n2” repeating units, wherein X1 is “SGGQQ,” “GAGQQ,” “GQGPY,” “AGQQ” or “SQ,” n1 is 4-8 and n2 is 6-10 and some positions may be absent
  SITE (4)..(67): This region may encompass 4-8 repeating “GPG-X1” repeating units, wherein X1 is “SGGQQ,” “GAGQQ,” “GQGPY,” “AGQQ” or “SQ,” and some positions may be absent
  SITE (7)..(11): This region may encompass “SGGQQ,” “GAGQQ,” “GQGPY,” “AGQQ” or “SQ,” wherein some positions may be absent
  SITE (15)..(19): This region may encompass “SGGQQ,” “GAGQQ,” “GQGPY,” “AGQQ” or “SQ,” wherein some positions may be absent
  SITE (23)..(27): This region may encompass “SGGQQ,” “GAGQQ,” “GQGPY,” “AGQQ” or “SQ,” wherein some positions may be absent
  SITE (31)..(35): This region may encompass “SGGQQ,” “GAGQQ,” “GQGPY,” “AGQQ” or “SQ,” wherein some positions may be absent
  SITE (39)..(43): This region may encompass “SGGQQ,” “GAGQQ,” “GQGPY,” “AGQQ” or “SQ,” wherein some positions may be absent
  SITE (47)..(51): This region may encompass “SGGQQ,” “GAGQQ,” “GQGPY,” “AGQQ” or “SQ,” wherein some positions may be absent
  SITE (55)..(59): This region may encompass “SGGQQ,” “GAGQQ,” “GQGPY,” “AGQQ” or “SQ,” wherein some positions may be absent
  SITE (63)..(67): This region may encompass “SGGQQ,” “GAGQQ,” “GQGPY,” “AGQQ” or “SQ,” wherein some positions may be absent
  SITE (71)..(80): This region may encompass 6-10 residues
  SITE (84)..(147): This region may encompass 4-8 repeating “GPG-X1” repeating units, wherein X1 is “SGGQQ,” “GAGQQ,” “GQGPY,” “AGQQ” or “SQ,” and some positions may be absent
  SITE (87)..(91): This region may encompass “SGGQQ,” “GAGQQ,” “GQGPY,” “AGQQ” or “SQ,” wherein some positions may be absent
  SITE (95)..(99): This region may encompass “SGGQQ,” “GAGQQ,” “GQGPY,” “AGQQ” or “SQ,” wherein some positions may be absent
  SITE (103)..(107): This region may encompass “SGGQQ,” “GAGQQ,” “GQGPY,” “AGQQ” or “SQ,” wherein some positions may be absent
  SITE (111)..(115): This region may encompass “SGGQQ,” “GAGQQ,” “GQGPY,” “AGQQ” or “SQ,” wherein some positions may be absent
  SITE (119)..(123): This region may encompass “SGGQQ,” “GAGQQ,” “GQGPY,” “AGQQ” or “SQ,” wherein some positions may be absent
  SITE (127)..(131): This region may encompass “SGGQQ,” “GAGQQ,” “GQGPY,” “AGQQ” or “SQ,” wherein some positions may be absent
  SITE (135)..(139): This region may encompass “SGGQQ,” “GAGQQ,” “GQGPY,” “AGQQ” or “SQ,” wherein some positions may be absent
  SITE (143)..(147): This region may encompass “SGGQQ,” “GAGQQ,” “GQGPY,” “AGQQ” or “SQ,” wherein some positions may be absent
  SITE (151)..(160): This region may encompass 6-10 residues
  SITE (164)..(227): This region may encompass 4-8 repeating “GPG-X1” repeating units, wherein X1 is “SGGQQ,” “GAGQQ,” “GQGPY,” “AGQQ” or “SQ,” and some positions may be absent
  SITE (167)..(171): This region may encompass “SGGQQ,” “GAGQQ,” “GQGPY,” “AGQQ” or “SQ,” wherein some positions may be absent
  SITE (175)..(179): This region may encompass “SGGQQ,” “GAGQQ,” “GQGPY,” “AGQQ” or “SQ,” wherein some positions may be absent
  SITE (183)..(187): This region may encompass “SGGQQ,” “GAGQQ,” “GQGPY,” “AGQQ” or “SQ,” wherein some positions may be absent
  SITE (191)..(195): This region may encompass “SGGQQ,” “GAGQQ,” “GQGPY,” “AGQQ” or “SQ,” wherein some positions may be absent
  SITE (199)..(203): This region may encompass “SGGQQ,” “GAGQQ,” “GQGPY,” “AGQQ” or “SQ,” wherein some positions may be absent
  SITE (207)..(211): This region may encompass “SGGQQ,” “GAGQQ,” “GQGPY,” “AGQQ” or “SQ,” wherein some positions may be absent
  SITE (215)..(219): This region may encompass “SGGQQ,” “GAGQQ,” “GQGPY,” “AGQQ” or “SQ,” wherein some positions may be absent
  SITE (223)..(227): This region may encompass “SGGQQ,” “GAGQQ,” “GQGPY,” “AGQQ” or “SQ,” wherein some positions may be absent
  SITE (231)..(240): This region may encompass 6-10 residues
  SITE (244)..(307): This region may encompass 4-8 repeating “GPG-X1” repeating units, wherein X1 is “SGGQQ,” “GAGQQ,” “GQGPY,” “AGQQ” or “SQ,” and some positions may be absent
  SITE (247)..(251): This region may encompass “SGGQQ,” “GAGQQ,” “GQGPY,” “AGQQ” or “SQ,” wherein some positions may be absent
  SITE (255)..(259): This region may encompass “SGGQQ,” “GAGQQ,” “GQGPY,” “AGQQ” or “SQ,” wherein some positions may be absent
  SITE (263)..(267): This region may encompass “SGGQQ,” “GAGQQ,” “GQGPY,” “AGQQ” or “SQ,” wherein some positions may be absent
  SITE (271)..(275): This region may encompass “SGGQQ,” “GAGQQ,” “GQGPY,” “AGQQ” or “SQ,” wherein some positions may be absent
  SITE (279)..(283): This region may encompass “SGGQQ,” “GAGQQ,” “GQGPY,” “AGQQ” or “SQ,” wherein some positions may be absent
  SITE (287)..(291): This region may encompass “SGGQQ,” “GAGQQ,” “GQGPY,” “AGQQ” or “SQ,” wherein some positions may be absent
  SITE (295)..(299): This region may encompass “SGGQQ,” “GAGQQ,” “GQGPY,” “AGQQ” or “SQ,” wherein some positions may be absent
  SITE (303)..(307): This region may encompass “SGGQQ,” “GAGQQ,” “GQGPY,” “AGQQ” or “SQ,” wherein some positions may be absent
  SITE (311)..(320): This region may encompass 6-10 residues
  SITE (324)..(387): This region may encompass 4-8 repeating “GPG-X1” repeating units, wherein X1 is “SGGQQ,” “GAGQQ,” “GQGPY,” “AGQQ” or “SQ,” and some positions may be absent
  SITE (327)..(331): This region may encompass “SGGQQ,” “GAGQQ,” “GQGPY,” “AGQQ” or “SQ,” wherein some positions may be absent
  SITE (335)..(339): This region may encompass “SGGQQ,” “GAGQQ,” “GQGPY,” “AGQQ” or “SQ,” wherein some positions may be absent
  SITE (343)..(347): This region may encompass “SGGQQ,” “GAGQQ,” “GQGPY,” “AGQQ” or “SQ,” wherein some positions may be absent
  SITE (351)..(355): This region may encompass “SGGQQ,” “GAGQQ,” “GQGPY,” “AGQQ” or “SQ,” wherein some positions may be absent
  SITE (359)..(363): This region may encompass “SGGQQ,” “GAGQQ,” “GQGPY,” “AGQQ” or “SQ,” wherein some positions may be absent
  SITE (367)..(371): This region may encompass “SGGQQ,” “GAGQQ,” “GQGPY,” “AGQQ” or “SQ,” wherein some positions may be absent
  SITE (375)..(379): This region may encompass “SGGQQ,” “GAGQQ,” “GQGPY,” “AGQQ” or “SQ,” wherein some positions may be absent
  SITE (383)..(387): This region may encompass “SGGQQ,” “GAGQQ,” “GQGPY,” “AGQQ” or “SQ,” wherein some positions may be absent
  SITE (391)..(400): This region may encompass 6-10 residues
  SITE (404)..(467): This region may encompass 4-8 repeating “GPG-X1” repeating units, wherein X1 is “SGGQQ,” “GAGQQ,” “GQGPY,” “AGQQ” or “SQ,” and some positions may be absent
  SITE (407)..(411): This region may encompass “SGGQQ,” “GAGQQ,” “GQGPY,” “AGQQ” or “SQ,” wherein some positions may be absent
  SITE (415)..(419): This region may encompass “SGGQQ,” “GAGQQ,” “GQGPY,” “AGQQ” or “SQ,” wherein some positions may be absent
  SITE (423)..(427): This region may encompass “SGGQQ,” “GAGQQ,” “GQGPY,” “AGQQ” or “SQ,” wherein some positions may be absent
  SITE (431)..(435): This region may encompass “SGGQQ,” “GAGQQ,” “GQGPY,” “AGQQ” or “SQ,” wherein some positions may be absent
  SITE (439)..(443): This region may encompass “SGGQQ,” “GAGQQ,” “GQGPY,” “AGQQ” or “SQ,” wherein some positions may be absent
  SITE (447)..(451): This region may encompass “SGGQQ,” “GAGQQ,” “GQGPY,” “AGQQ” or “SQ,” wherein some positions may be absent
  SITE (455)..(459): This region may encompass “SGGQQ,” “GAGQQ,” “GQGPY,” “AGQQ” or “SQ,” wherein some positions may be absent
  SITE (463)..(467): This region may encompass “SGGQQ,” “GAGQQ,” “GQGPY,” “AGQQ” or “SQ,” wherein some positions may be absent
  SITE (471)..(480): This region may encompass 6-10 residues
  SITE (484)..(547): This region may encompass 4-8 repeating “GPG-X1” repeating units, wherein X1 is “SGGQQ,” “GAGQQ,” “GQGPY,” “AGQQ” or “SQ,” and some positions may be absent
  SITE (487)..(491): This region may encompass “SGGQQ,” “GAGQQ,” “GQGPY,” “AGQQ” or “SQ,” wherein some positions may be absent
  SITE (495)..(499): This region may encompass “SGGQQ,” “GAGQQ,” “GQGPY,” “AGQQ” or “SQ,” wherein some positions may be absent
  SITE (503)..(507): This region may encompass “SGGQQ,” “GAGQQ,” “GQGPY,” “AGQQ” or “SQ,” wherein some positions may be absent
  SITE (511)..(515): This region may encompass “SGGQQ,” “GAGQQ,” “GQGPY,” “AGQQ” or “SQ,” wherein some positions may be absent
  SITE (519)..(523): This region may encompass “SGGQQ,” “GAGQQ,” “GQGPY,” “AGQQ” or “SQ,” wherein some positions may be absent
  SITE (527)..(531): This region may encompass “SGGQQ,” “GAGQQ,” “GQGPY,” “AGQQ” or “SQ,” wherein some positions may be absent
  SITE (535)..(539): This region may encompass “SGGQQ,” “GAGQQ,” “GQGPY,” “AGQQ” or “SQ,” wherein some positions may be absent
  SITE (543)..(547): This region may encompass “SGGQQ,” “GAGQQ,” “GQGPY,” “AGQQ” or “SQ,” wherein some positions may be absent
  SITE (551)..(560): This region may encompass 6-10 residues
  SITE (564)..(627): This region may encompass 4-8 repeating “GPG-X1” repeating units, wherein X1 is “SGGQQ,” “GAGQQ,” “GQGPY,” “AGQQ” or “SQ,” and some positions may be absent
  SITE (567)..(571): This region may encompass “SGGQQ,” “GAGQQ,” “GQGPY,” “AGQQ” or “SQ,” wherein some positions may be absent
  SITE (575)..(579): This region may encompass “SGGQQ,” “GAGQQ,” “GQGPY,” “AGQQ” or “SQ,” wherein some positions may be absent
  SITE (583)..(587): This region may encompass “SGGQQ,” “GAGQQ,” “GQGPY,” “AGQQ” or “SQ,” wherein some positions may be absent
  SITE (591)..(595): This region may encompass “SGGQQ,” “GAGQQ,” “GQGPY,” “AGQQ” or “SQ,” wherein some positions may be absent
  SITE (599)..(603): This region may encompass “SGGQQ,” “GAGQQ,” “GQGPY,” “AGQQ” or “SQ,” wherein some positions may be absent
  SITE (607)..(611): This region may encompass “SGGQQ,” “GAGQQ,” “GQGPY,” “AGQQ” or “SQ,” wherein some positions may be absent
  SITE (615)..(619): This region may encompass “SGGQQ,” “GAGQQ,” “GQGPY,” “AGQQ” or “SQ,” wherein some positions may be absent
  SITE (623)..(627): This region may encompass “SGGQQ,” “GAGQQ,” “GQGPY,” “AGQQ” or “SQ,” wherein some positions may be absent
  SITE (631)..(640): This region may encompass 6-10 residues
  SITE (644)..(707): This region may encompass 4-8 repeating “GPG-X1” repeating units, wherein X1 is “SGGQQ,” “GAGQQ,” “GQGPY,” “AGQQ” or “SQ,” and some positions may be absent
  SITE (647)..(651): This region may encompass “SGGQQ,” “GAGQQ,” “GQGPY,” “AGQQ” or “SQ,” wherein some positions may be absent
  SITE (655)..(659): This region may encompass “SGGQQ,” “GAGQQ,” “GQGPY,” “AGQQ” or “SQ,” wherein some positions may be absent
  SITE (663)..(667): This region may encompass “SGGQQ,” “GAGQQ,” “GQGPY,” “AGQQ” or “SQ,” wherein some positions may be absent
  SITE (671)..(675): This region may encompass “SGGQQ,” “GAGQQ,” “GQGPY,” “AGQQ” or “SQ,” wherein some positions may be absent
  SITE (679)..(683): This region may encompass “SGGQQ,” “GAGQQ,” “GQGPY,” “AGQQ” or “SQ,” wherein some positions may be absent
  SITE (687)..(691): This region may encompass “SGGQQ,” “GAGQQ,” “GQGPY,” “AGQQ” or “SQ,” wherein some positions may be absent
  SITE (695)..(699): This region may encompass “SGGQQ,” “GAGQQ,” “GQGPY,” “AGQQ” or “SQ,” wherein some positions may be absent
  SITE (703)..(707): This region may encompass “SGGQQ,” “GAGQQ,” “GQGPY,” “AGQQ” or “SQ,” wherein some positions may be absent
  SITE (711)..(720): This region may encompass 6-10 residues
  SITE (724)..(787): This region may encompass 4-8 repeating “GPG-X1” repeating units, wherein X1 is “SGGQQ,” “GAGQQ,” “GQGPY,” “AGQQ” or “SQ,” and some positions may be absent
  SITE (727)..(731): This region may encompass “SGGQQ,” “GAGQQ,” “GQGPY,” “AGQQ” or “SQ,” wherein some positions may be absent
  SITE (735)..(739): This region may encompass “SGGQQ,” “GAGQQ,” “GQGPY,” “AGQQ” or “SQ,” wherein some positions may be absent
  SITE (743)..(747): This region may encompass “SGGQQ,” “GAGQQ,” “GQGPY,” “AGQQ” or “SQ,” wherein some positions may be absent
  SITE (751)..(755): This region may encompass “SGGQQ,” “GAGQQ,” “GQGPY,” “AGQQ” or “SQ,” wherein some positions may be absent
  SITE (759)..(763): This region may encompass “SGGQQ,” “GAGQQ,” “GQGPY,” “AGQQ” or “SQ,” wherein some positions may be absent
  SITE (767)..(771): This region may encompass “SGGQQ,” “GAGQQ,” “GQGPY,” “AGQQ” or “SQ,” wherein some positions may be absent
  SITE (775)..(779): This region may encompass “SGGQQ,” “GAGQQ,” “GQGPY,” “AGQQ” or “SQ,” wherein some positions may be absent
  SITE (783)..(787): This region may encompass “SGGQQ,” “GAGQQ,” “GQGPY,” “AGQQ” or “SQ,” wherein some positions may be absent
  SITE (791)..(800): This region may encompass 6-10 residues
  SITE (804)..(867): This region may encompass 4-8 repeating “GPG-X1” repeating units, wherein X1 is “SGGQQ,” “GAGQQ,” “GQGPY,” “AGQQ” or “SQ,” and some positions may be absent
  SITE (807)..(811): This region may encompass “SGGQQ,” “GAGQQ,” “GQGPY,” “AGQQ” or “SQ,” wherein some positions may be absent
  SITE (815)..(819): This region may encompass “SGGQQ,” “GAGQQ,” “GQGPY,” “AGQQ” or “SQ,” wherein some positions may be absent
  SITE (823)..(827): This region may encompass “SGGQQ,” “GAGQQ,” “GQGPY,” “AGQQ” or “SQ,” wherein some positions may be absent
  SITE (831)..(835): This region may encompass “SGGQQ,” “GAGQQ,” “GQGPY,” “AGQQ” or “SQ,” wherein some positions may be absent
  SITE (839)..(843): This region may encompass “SGGQQ,” “GAGQQ,” “GQGPY,” “AGQQ” or “SQ,” wherein some positions may be absent
  SITE (847)..(851): This region may encompass “SGGQQ,” “GAGQQ,” “GQGPY,” “AGQQ” or “SQ,” wherein some positions may be absent
  SITE (855)..(859): This region may encompass “SGGQQ,” “GAGQQ,” “GQGPY,” “AGQQ” or “SQ,” wherein some positions may be absent
  SITE (863)..(867): This region may encompass “SGGQQ,” “GAGQQ,” “GQGPY,” “AGQQ” or “SQ,” wherein some positions may be absent
  SITE (871)..(880): This region may encompass 6-10 residues
  SITE (884)..(947): This region may encompass 4-8 repeating “GPG-X1” repeating units, wherein X1 is “SGGQQ,” “GAGQQ,” “GQGPY,” “AGQQ” or “SQ,” and some positions may be absent
  SITE (887)..(891): This region may encompass “SGGQQ,” “GAGQQ,” “GQGPY,” “AGQQ” or “SQ,” wherein some positions may be absent
  SITE (895)..(899): This region may encompass “SGGQQ,” “GAGQQ,” “GQGPY,” “AGQQ” or “SQ,” wherein some positions may be absent
  SITE (903)..(907): This region may encompass “SGGQQ,” “GAGQQ,” “GQGPY,” “AGQQ” or “SQ,” wherein some positions may be absent
  SITE (911)..(915): This region may encompass “SGGQQ,” “GAGQQ,” “GQGPY,” “AGQQ” or “SQ,” wherein some positions may be absent
  SITE (919)..(923): This region may encompass “SGGQQ,” “GAGQQ,” “GQGPY,” “AGQQ” or “SQ,” wherein some positions may be absent
  SITE (927)..(931): This region may encompass “SGGQQ,” “GAGQQ,” “GQGPY,” “AGQQ” or “SQ,” wherein some positions may be absent
  SITE (935)..(939): This region may encompass “SGGQQ,” “GAGQQ,” “GQGPY,” “AGQQ” or “SQ,” wherein some positions may be absent
  SITE (943)..(947): This region may encompass “SGGQQ,” “GAGQQ,” “GQGPY,” “AGQQ” or “SQ,” wherein some positions may be absent
  SITE (951)..(960): This region may encompass 6-10 residues
  SITE (964)..(1027): This region may encompass 4-8 repeating “GPG-X1” repeating units, wherein X1 is “SGGQQ,” “GAGQQ,” “GQGPY,” “AGQQ” or “SQ,” and some positions may be absent
  SITE (967)..(971): This region may encompass “SGGQQ,” “GAGQQ,” “GQGPY,” “AGQQ” or “SQ,” wherein some positions may be absent
  SITE (975)..(979): This region may encompass “SGGQQ,” “GAGQQ,” “GQGPY,” “AGQQ” or “SQ,” wherein some positions may be absent
  SITE (983)..(987): This region may encompass “SGGQQ,” “GAGQQ,” “GQGPY,” “AGQQ” or “SQ,” wherein some positions may be absent
  SITE (991)..(995): This region may encompass “SGGQQ,” “GAGQQ,” “GQGPY,” “AGQQ” or “SQ,” wherein some positions may be absent
  SITE (999)..(1003): This region may encompass “SGGQQ,” “GAGQQ,” “GQGPY,” “AGQQ” or “SQ,” wherein some positions may be absent
  SITE (1007)..(1011): This region may encompass “SGGQQ,” “GAGQQ,” “GQGPY,” “AGQQ” or “SQ,” wherein some positions may be absent
  SITE (1015)..(1019): This region may encompass “SGGQQ,” “GAGQQ,” “GQGPY,” “AGQQ” or “SQ,” wherein some positions may be absent
  SITE (1023)..(1027): This region may encompass “SGGQQ,” “GAGQQ,” “GQGPY,” “AGQQ” or “SQ,” wherein some positions may be absent
  SITE (1031)..(1040): This region may encompass 6-10 residues
  SITE (1044)..(1107): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1047)..(1051): This region may encompass “SGGQQ,” “GAGQQ,” “GQGPY,” “AGQQ” or “SQ,” wherein some positions may be absent
  SITE (1055)..(1059): This region may encompass “SGGQQ,” “GAGQQ,” “GQGPY,” “AGQQ” or “SQ,” wherein some positions may be absent
  SITE (1063)..(1067): This region may encompass “SGGQQ,” “GAGQQ,” “GQGPY,” “AGQQ” or “SQ,” wherein some positions may be absent
  SITE (1071)..(1075): This region may encompass “SGGQQ,” “GAGQQ,” “GQGPY,” “AGQQ” or “SQ,” wherein some positions may be absent
  SITE (1079)..(1083): This region may encompass “SGGQQ,” “GAGQQ,” “GQGPY,” “AGQQ” or “SQ,” wherein some positions may be absent
  SITE (1087)..(1091): This region may encompass “SGGQQ,” “GAGQQ,” “GQGPY,” “AGQQ” or “SQ,” wherein some positions may be absent
  SITE (1095)..(1099): This region may encompass “SGGQQ,” “GAGQQ,” “GQGPY,” “AGQQ” or “SQ,” wherein some positions may be absent
  SITE (1103)..(1107): This region may encompass “SGGQQ,” “GAGQQ,” “GQGPY,” “AGQQ” or “SQ,” wherein some positions may be absent
  SITE (1111)..(1120): This region may encompass 6-10 residues
  SITE (1124)..(1187): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1127)..(1131): This region may encompass “SGGQQ,” “GAGQQ,” “GQGPY,” “AGQQ” or “SQ,” wherein some positions may be absent
  SITE (1135)..(1139): This region may encompass “SGGQQ,” “GAGQQ,” “GQGPY,” “AGQQ” or “SQ,” wherein some positions may be absent
  SITE (1143)..(1147): This region may encompass “SGGQQ,” “GAGQQ,” “GQGPY,” “AGQQ” or “SQ,” wherein some positions may be absent
  SITE (1151)..(1155): This region may encompass “SGGQQ,” “GAGQQ,” “GQGPY,” “AGQQ” or “SQ,” wherein some positions may be absent
  SITE (1159)..(1163): This region may encompass “SGGQQ,” “GAGQQ,” “GQGPY,” “AGQQ” or “SQ,” wherein some positions may be absent
  SITE (1167)..(1171): This region may encompass “SGGQQ,” “GAGQQ,” “GQGPY,” “AGQQ” or “SQ,” wherein some positions may be absent
  SITE (1175)..(1179): This region may encompass “SGGQQ,” “GAGQQ,” “GQGPY,” “AGQQ” or “SQ,” wherein some positions may be absent
  SITE (1183)..(1187): This region may encompass “SGGQQ,” “GAGQQ,” “GQGPY,” “AGQQ” or “SQ,” wherein some positions may be absent
  SITE (1191)..(1200): This region may encompass 6-10 residues
  SITE (1204)..(1267): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1207)..(1211): This region may encompass “SGGQQ,” “GAGQQ,” “GQGPY,” “AGQQ” or “SQ,” wherein some positions may be absent
  SITE (1215)..(1219): This region may encompass “SGGQQ,” “GAGQQ,” “GQGPY,” “AGQQ” or “SQ,” wherein some positions may be absent
  SITE (1223)..(1227): This region may encompass “SGGQQ,” “GAGQQ,” “GQGPY,” “AGQQ” or “SQ,” wherein some positions may be absent
  SITE (1231)..(1235): This region may encompass “SGGQQ,” “GAGQQ,” “GQGPY,” “AGQQ” or “SQ,” wherein some positions may be absent
  SITE (1239)..(1243): This region may encompass “SGGQQ,” “GAGQQ,” “GQGPY,” “AGQQ” or “SQ,” wherein some positions may be absent
  SITE (1247)..(1251): This region may encompass “SGGQQ,” “GAGQQ,” “GQGPY,” “AGQQ” or “SQ,” wherein some positions may be absent
  SITE (1255)..(1259): This region may encompass “SGGQQ,” “GAGQQ,” “GQGPY,” “AGQQ” or “SQ,” wherein some positions may be absent
  SITE (1263)..(1267): This region may encompass “SGGQQ,” “GAGQQ,” “GQGPY,” “AGQQ” or “SQ,” wherein some positions may be absent
  SITE (1271)..(1280): This region may encompass 6-10 residues
  SITE (1284)..(1347): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1287)..(1291): This region may encompass “SGGQQ,” “GAGQQ,” “GQGPY,” “AGQQ” or “SQ,” wherein some positions may be absent
  SITE (1295)..(1299): This region may encompass “SGGQQ,” “GAGQQ,” “GQGPY,” “AGQQ” or “SQ,” wherein some positions may be absent
  SITE (1303)..(1307): This region may encompass “SGGQQ,” “GAGQQ,” “GQGPY,” “AGQQ” or “SQ,” wherein some positions may be absent
  SITE (1311)..(1315): This region may encompass “SGGQQ,” “GAGQQ,” “GQGPY,” “AGQQ” or “SQ,” wherein some positions may be absent
  SITE (1319)..(1323): This region may encompass “SGGQQ,” “GAGQQ,” “GQGPY,” “AGQQ” or “SQ,” wherein some positions may be absent
  SITE (1327)..(1331): This region may encompass “SGGQQ,” “GAGQQ,” “GQGPY,” “AGQQ” or “SQ,” wherein some positions may be absent
  SITE (1335)..(1339): This region may encompass “SGGQQ,” “GAGQQ,” “GQGPY,” “AGQQ” or “SQ,” wherein some positions may be absent
  SITE (1343)..(1347): This region may encompass “SGGQQ,” “GAGQQ,” “GQGPY,” “AGQQ” or “SQ,” wherein some positions may be absent
  SITE (1351)..(1360): This region may encompass 6-10 residues
  SITE (1364)..(1427): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1367)..(1371): This region may encompass “SGGQQ,” “GAGQQ,” “GQGPY,” “AGQQ” or “SQ,” wherein some positions may be absent
  SITE (1375)..(1379): This region may encompass “SGGQQ,” “GAGQQ,” “GQGPY,” “AGQQ” or “SQ,” wherein some positions may be absent
  SITE (1383)..(1387): This region may encompass “SGGQQ,” “GAGQQ,” “GQGPY,” “AGQQ” or “SQ,” wherein some positions may be absent
  SITE (1391)..(1395): This region may encompass “SGGQQ,” “GAGQQ,” “GQGPY,” “AGQQ” or “SQ,” wherein some positions may be absent
  SITE (1399)..(1403): This region may encompass “SGGQQ,” “GAGQQ,” “GQGPY,” “AGQQ” or “SQ,” wherein some positions may be absent
  SITE (1407)..(1411): This region may encompass “SGGQQ,” “GAGQQ,” “GQGPY,” “AGQQ” or “SQ,” wherein some positions may be absent
  SITE (1415)..(1419): This region may encompass “SGGQQ,” “GAGQQ,” “GQGPY,” “AGQQ” or “SQ,” wherein some positions may be absent
  SITE (1423)..(1427): This region may encompass “SGGQQ,” “GAGQQ,” “GQGPY,” “AGQQ” or “SQ,” wherein some positions may be absent
  SITE (1431)..(1440): This region may encompass 6-10 residues
  SITE (1444)..(1507): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1447)..(1451): This region may encompass “SGGQQ,” “GAGQQ,” “GQGPY,” “AGQQ” or “SQ,” wherein some positions may be absent
  SITE (1455)..(1459): This region may encompass “SGGQQ,” “GAGQQ,” “GQGPY,” “AGQQ” or “SQ,” wherein some positions may be absent
  SITE (1463)..(1467): This region may encompass “SGGQQ,” “GAGQQ,” “GQGPY,” “AGQQ” or “SQ,” wherein some positions may be absent
  SITE (1471)..(1475): This region may encompass “SGGQQ,” “GAGQQ,” “GQGPY,” “AGQQ” or “SQ,” wherein some positions may be absent
  SITE (1479)..(1483): This region may encompass “SGGQQ,” “GAGQQ,” “GQGPY,” “AGQQ” or “SQ,” wherein some positions may be absent
  SITE (1487)..(1491): This region may encompass “SGGQQ,” “GAGQQ,” “GQGPY,” “AGQQ” or “SQ,” wherein some positions may be absent
  SITE (1495)..(1499): This region may encompass “SGGQQ,” “GAGQQ,” “GQGPY,” “AGQQ” or “SQ,” wherein some positions may be absent
  SITE (1503)..(1507): This region may encompass “SGGQQ,” “GAGQQ,” “GQGPY,” “AGQQ” or “SQ,” wherein some positions may be absent
  SITE (1511)..(1520): This region may encompass 6-10 residues
  SITE (1524)..(1587): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1527)..(1531): This region may encompass “SGGQQ,” “GAGQQ,” “GQGPY,” “AGQQ” or “SQ,” wherein some positions may be absent
  SITE (1535)..(1539): This region may encompass “SGGQQ,” “GAGQQ,” “GQGPY,” “AGQQ” or “SQ,” wherein some positions may be absent
  SITE (1543)..(1547): This region may encompass “SGGQQ,” “GAGQQ,” “GQGPY,” “AGQQ” or “SQ,” wherein some positions may be absent
  SITE (1551)..(1555): This region may encompass “SGGQQ,” “GAGQQ,” “GQGPY,” “AGQQ” or “SQ,” wherein some positions may be absent
  SITE (1559)..(1563): This region may encompass “SGGQQ,” “GAGQQ,” “GQGPY,” “AGQQ” or “SQ,” wherein some positions may be absent
  SITE (1567)..(1571): This region may encompass “SGGQQ,” “GAGQQ,” “GQGPY,” “AGQQ” or “SQ,” wherein some positions may be absent
  SITE (1575)..(1579): This region may encompass “SGGQQ,” “GAGQQ,” “GQGPY,” “AGQQ” or “SQ,” wherein some positions may be absent
  SITE (1583)..(1587): This region may encompass “SGGQQ,” “GAGQQ,” “GQGPY,” “AGQQ” or “SQ,” wherein some positions may be absent
  SITE (1591)..(1600): This region may encompass 6-10 residues
GGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAA

SEQ ID NO: 1402 - protein, 5 aa, Artificial Sequence
  Synthetic peptide
SGGQQ

SEQ ID NO: 1403 - protein, 5 aa, Artificial Sequence
  Synthetic peptide
GAGQQ

SEQ ID NO: 1404 - protein, 5 aa, Artificial Sequence
  Synthetic peptide
GQGPY

SEQ ID NO: 1405 - protein, 4 aa, Artificial Sequence
  Synthetic peptide
AGQQ

SEQ ID NO: 1406 - protein, 8 aa, Artificial Sequence
  Synthetic His tag
  SITE (1)..(8): This sequence may encompass 6-8 residues
HHHHHHHH

(19) United States
(12) Patent Application Publication
(10) Pub. No.: US 2024/0050352 A1
(43) Pub. Date: Feb. 15, 2024

(54) RECOMBINANT SILK COMPOSITIONS AND METHODS OF MAKING THEREOF

(71) Applicant: BOLT THREADS, INC., Emeryville, CA (US)

(72) Inventors: Lindsay Wray, Emeryville, CA (US); Joshua Tyler Kittleson, Emeryville, CA (US)

(21) Appl. No.: 18/017,216

(22) PCT Filed: Jul. 16, 2021
(86) PCT No.: PCT/US21/42084

§ 371 Date: Jan. 20, 2023

Related U.S. Application Data
(60) Provisional application No. 63/055,894, filed on Jul. 23, 2020.

(51) Int. Cl. A61K 8/64; A61K 8/02; A61Q 19/00; A61Q 19/10

(52) U.S. Cl. CPC A61K 8/64; A61K 8/0279; A61Q 19/00; A61Q 19/10; A61K 2800/28

(57) ABSTRACT

Disclosed herein are recombinant spider silk compositions formed from a stable powder as it hydrates, cleanses, defends, detoxifies, mattifies, and/or exfoliates the skin, among other uses.

### CROSS REFERENCE TO RELATED APPLICATIONS

This application claims the benefit of U.S. Provisional Application No. 63/055,894, filed Jul. 23, 2020, of which is hereby incorporated in their entirety by reference.

### FIELD OF THE INVENTION

The present disclosure relates to recombinant spider silk compositions formed from a stable powder as it hydrates, cleanses, defends, detoxifies, mattifies, and/or exfoliates the skin, among other uses.

### BACKGROUND

Silk is a structural protein that has many qualities that make it desirable for use in applications such as skincare and cosmetics. Recent technology has resulted in the scalable production of various recombinant spider silk polypeptides and polypeptides that are derived from recombinant spider silk polypeptides using various host organisms. However, difficulties with hydrating recovered recombinant silk powder in a solution at a large scale to yield desirable formulations, such as full-length silk-based solid or semi-solid compositions has been a significant challenge.

Most cosmetics and skincare products that incorporate silk use silk that has been hydrolyzed into small amino acid chains. However, these compositions comprising fragments of degraded silk proteins lose the desirable properties of silk. Furthermore, use of harmful solvents is undesirable for use in silk formulations meant to contact the skin.

While new methods of producing sericin-depleted silkworm silk (referred to herein as “silk fibroin”) have resulted in various skincare products that do manage to incorporate full-length (i.e. non-hydrolyzed) silk proteins, the self-aggregation properties of silk can affect the shelf-stability of these products. Specifically, full-length silk fibroin molecules tend to aggregate and precipitate out of solution. Furthermore, these processes are not scalable, and thus are not commercially viable. Since recombinant spider silk polypeptides form similar secondary and tertiary structures to silk fibroin, it is equally desirable for use in cosmetics and skincare formulations but also can exhibit similar stability issues due to self-aggregation.

Therefore, what is needed are scalable methods of increasing the stability of recombinant spider silk polypeptides for shelf-life longevity (e.g. raw material storage) and stability in silk formulations (e.g., cosmetic and skincare formulations) in a variety of material forms that do not use harmful solvents, improve aesthetic value, and maintain the desirable properties of full-length silk proteins.

### SUMMARY

Provided herein, in some embodiments, is a method of making a silk-based composition, comprising: mixing a recombinant silk particle comprising a hollow core and a solvent, wherein the recombinant silk particle functions as a carrier for the solvent; thereby transforming the recombinant silk particle to the silk-based composition.

In some embodiments, the recombinant silk particle comprises an opening in said outer shell. In some embodiments, the recombinant spider particle is in the form of a dry powder. In some embodiments, the mixing said recombinant silk particle and solvent expands the hollow core.

In some embodiments, the solvent comprises an aqueous solvent, an alcohol, an oil-based solvent, or a silicone. In some embodiments, the solvent is water, glycerin, deionized water, olive oil, pentylene glycol, or silicone. In some embodiments, the recombinant silk particle is a carrier for the solvent.

In some embodiments, the recombinant spider silk particle swells when mixed with the solvent. In some embodiments, the diameter of the outer shell is from 5 μm to 25 μm when the recombinant silk particle is dry. In some embodiments, the diameter of the outer shell swells to up to 120 μm when mixed with the solvent. In some embodiments, the outer shell thickness is less than 20%, less than 15%, or less than 10% of the diameter of the recombinant silk particle.

In some embodiments, the composition comprises a plurality of recombinant silk particles. In some embodiments, the recombinant silk particles are present in said composition at a concentration of from 1% to 10% wt/wt in said solvent.

In some embodiments, the recombinant silk particle comprises recombinant spider silk. In some embodiments, the recombinant silk particle comprises a polypeptide, the polypeptide comprising SEQ ID NO.: 2. In some embodiments, the recombinant silk particle comprising a polypeptide, the polypeptide comprising at least two concatenated repeat units of SEQ ID NO.: 2.

In some embodiments, the recombinant silk particle comprises a concentration of at least 1% by weight of polypeptide.

In some embodiments, the recombinant silk particle is water insoluble. In some embodiments, the recombinant silk particle is a bead. In some embodiments, the powder is spray dried.

In some embodiments, the method of making a silk-based composition further comprises spray drying a composition comprising a recombinant silk polypeptide to form a dry powder comprising said recombinant silk particle. In some embodiments, the method of making a silk-based composition further comprises adding a dye to the silk-based composition or the recombinant silk particle. In some embodiments, the method of making a silk-based composition further comprises adding a surfactant or humectant to the silk-based composition or the recombinant silk particle.

In some embodiments, the silk-based composition is a cosmetic or skincare formulation. In some embodiments, the silk-based composition improves firming, elasticity, overall skin health, wound healing, and/or appearance of the skin.

In some embodiments, application of the silk-based composition to the skin reduces oxidative stress. In some embodiments, the oxidative stress is selected from the group consisting of: basal level of oxidative stress, oxidative stress caused by blue light irradiation, pollution induced oxidative stress, UVA induced oxidative stress, and UVB oxidative stress. In some embodiments, application of the silk-based composition to skin mattifies the surface of the skin.

Also provided herein, in some embodiments, is a method of making a silk-based composition, comprising: mixing a recombinant silk particle comprising a hollow core and a solvent, wherein the recombinant silk particle is a carrier for the solvent, and wherein the recombinant silk particle comprising a polypeptide, the polypeptide comprising at least two concatenated repeat units of SEQ ID NO.: 2, thereby forming the silk-based composition.

Also provided herein, in some embodiments, is a method of making a silk-based solid or hydrogel, comprising: mixing a recombinant silk particle comprising a hollow core and a solvent, wherein the recombinant silk particle functions as a carrier for the solvent, thereby forming a silk-based composition; applying the silk-based composition to a surface; and drying the silk-based composition to form the silk-based solid or hydrogel.

In some embodiments, the surface comprises skin, hair, or nails. In some embodiments, said dried silk-based composition forms a barrier on said surface. In some embodiments, the barrier is substantially homogenous.

In some embodiments, the silk-based solid or hydrogel is a bead. In some embodiments, the silk-based solid or hydrogel is a film. In some embodiments, the silk-based solid or hydrogel is a cosmetic or skincare formulation.

Also provided herein, in some embodiments, is a method of making a silk-based formulation, comprising: providing a silk-based formulation comprising a silk protein powder and a solvent, wherein the recombinant silk powder comprises a hollow core and is a carrier for the solvent. In some embodiments, the recombinant silk powder is a carrier for the solvent.

In some embodiments, the method of making a silk-based formulation further comprises adding a dye to the silk-based composition or the recombinant silk particle. In some embodiments, the method of making a silk-based formulation further comprises drying the silk-based formulation to form a silk-based solid or hydrogel.

In some embodiments, the method of making a silk-based formulation further comprises mixing the silk-based formulation into an emulsion to form a silk-based emulsion. In some embodiments the method of making a silk-based formulation further comprises drying the silk-based emulsion to form a silk-based solid or hydrogel.

In some embodiments, the method of making a silk-based formulation further comprises mixing an additive and the silk-based solid or hydrogel to form an enriched silk-based formulation. In some embodiments, the method of making a silk-based formulation further comprises coagulating the silk-based formulation to form aggregated silk in the silk-based formulation.

In some embodiments, the silk-based formulation comprises a gel phase. In some embodiments, the silk protein powder comprises recombinant spider silk. In some embodiments, the recombinant spider silk comprises full length silk proteins. In some embodiments, the silk-based formulation is a skincare or cosmetic formulation. In some embodiments, the alcohol is glycerol. In some embodiments, the oil-based solvent comprises a free fatty acid. In some embodiments, the free fatty acid comprises olive oil, grape-seed oil, or a triglyceride. In some embodiments, the silk-based formulation disperses upon contact with skin or water or gentle rubbing.

Also provided herein, in some embodiments, is a composition comprising a recombinant silk particle comprising an outer shell and a hollow core. In some embodiments, the recombinant silk particle is adapted to form a carrier for a solvent. In some embodiments, the recombinant silk particle is in powder form. In some embodiments, the recombinant silk particle comprises recombinant spider silk.

In some embodiments, the composition exfoliates the skin. In some embodiments, the composition further comprises a dye.

Also provided herein, in some embodiments, is a composition comprising a recombinant silk particle and a solvent, wherein the recombinant silk particle comprises an outer shell and a hollow core. In some embodiments, the recombinant silk particle is a carrier for the solvent.

In some embodiments, the composition comprises a surfactant or humectant. In some embodiments, the hollow core is expanded by the solvent. In some embodiments, the composition is a cosmetic or skincare formulation. In some embodiments, the composition cleanses the skin.

Also provided herein, in some embodiments, is a silk cosmetic or skincare product comprising a silk protein particle a solvent, wherein the silk protein particle comprises a hollow core and carries the solvent.

In some embodiments, the silk protein particle is water insoluble. In some embodiments, the silk cosmetic or skincare product is a solid, a hydrogel, or a film.

Also provided herein, in some embodiments, is a recombinant silk cosmetic or skincare product comprising a semi-solid, wherein the semi-solid comprises dispersed non-aggregated recombinant silk protein and a solvent.

In some embodiments, the semi-solid removes residues upon contact with skin. In some embodiments, the semi-solid is a hydrogel.

Also provided herein, in some embodiments, is a composition comprising a recombinant silk particle comprising a hollow core. In some embodiments, the recombinant silk particle is an exfoliant.

Also provided herein, according to some embodiments, is a method of improving the appearance of skin comprising applying to the skin a composition comprising a recombinant silk particle comprising a hollow core. In some embodiments, the composition comprises about 1 wt % recombinant silk protein.

In some embodiments, the improved appearance of skin provides at least one result selected from the group comprising: increasing skin firmness/plumpness, increasing elasticity, improving overall skin health, increasing hydration, improving wound healing, reducing oxidative stress levels, attenuating pollution induced oxidative stress, attenuating UVA or UVB induced oxidative stress, and any combination thereof.

In some embodiments, provided herein is a method of cleansing a surface, comprising applying a composition comprising a recombinant silk particle comprising a hollow core to a surface to form a film or bead; and removing the film or bead from the surface.

Also provided herein, according to some embodiments, is a method of making a silk-based composition, comprising drying a composition comprising recombinant silk to form a dried powder comprising recombinant silk particles. In some embodiments, the recombinant silk particles comprise an outer shell and a hollow core.

Also provided herein, according to some embodiments, is a composition comprising a dried powder comprising a recombinant silk protein. In some embodiments, the dried powder comprises recombinant silk particles comprising a hollow core and an outer shell. In some embodiments, the recombinant silk particles are adapted to act as a carrier.

### BRIEF DESCRIPTION OF THE DRAWINGS

The foregoing and other objects, features and advantages will be apparent from the following description of particular embodiments of the invention, as illustrated in the accompanying drawings.

FIG. 1A shows scanning electron microscope (SEM) images of intact and cracked recombinant silk powder particles with 18B polypeptide sequences (SEQ ID NO: 1) (“18B powder” or “18B”) in the dry state. FIG. 1B shows a hollow shell morphology in the hydrated state via light and polarized microscopy.

FIG. 2A shows light microscopy images of 18B powder resuspended in various different solvents. FIG. 2B shows an image of 1 g of 18B powder in a dry state and 1 g of 18B powder after saturated exposure to an aqueous solution.

FIG. 3A shows a mixture of water, acid textile dye, and 18B powder generated according to various embodiments of the present invention. FIG. 3B shows dyed 18B powder at the final powder state and after being applied to skin. FIG. 3C shows different concentrations of dyed 18B powder added to cream emulsions. FIG. 3D shows the stability of color fastness of dyed 18B powder after 6 months of storage at 4° C.

FIG. 4 shows a schematic diagram of an 18B powder solution being applied to the skin, drying, and forming a thin homogenous barrier on the skin surface of the epidermal layer.

FIG. 5A shows a schematic diagram of adding an 18B powder solution to a substrate according to various embodiments of the present invention, and SEM images of dried 1 wt % 18B powder solution coalescing into a thin film of about 1 μm thickness when applied to the substrate at a mass per surface area of 2 mg/cm². FIG. 5B shows the thickness of film changing depending on the solution concentration, volume, and surface area. This image represents various difference masses of a 1 wt % solution dispensed onto a 4 cm² area.

FIG. 5C shows images of the skin before and after dyed 2 wt % 18B powder solution has been applied at 2 mg/cm² and dried down (5 mins of drying at ambient conditions of 21° C. and 40% humidity.

FIG. 6A shows images of an 18B powder protein barrier being visualized by fluorescently tagging the protein. FIG. 6B shows an experimental design to investigate the effect of repeated abrasion on an 18B powder protein barrier. FIG. 6C shows images of an 18B powder protein barrier subjected to repeated abrasion of no rubs, 100 rubs, and 600 rubs, as compared to bare skin (“control”). FIG. 6D shows images of an 18B powder protein barrier on the skin after one to five passes of a wet wipe. FIG. 6E shows images of the wet wipe after multiple passes.

FIGS. 7A and 7B show the results of a pollution rating study to investigate the effects of an 18B powder solution on carbon particles. FIG. 7C shows images of pollution washes performed on polyurethane material or faux skin using hydrolyzed silk and an 18B powder solution, as compared to a control. FIG. 7D shows images of pollution washes performed on hair using 1% and 2% 18B powder solution, as compared to an untreated control, and the resultant rinse water after the washings.

FIG. 8A shows images of various dry substances including 18B powder, charcoal black, and rice bran, rubbed on the skin over black eyeshadow and images after a water rinse. FIG. 8B shows microscopic images of 18B powder used as an exfoliant on a skin mimic, as compared to a control and other standard ingredients. FIG. 8C shows a 10 wt % 18B powder solution used as a cleanser on a skin substitute, as compared to a control and hydrolyzed silk solutions. FIG. 8D shows various concentrations of an 18B powder solution used as cleanser additive, as compared to a cleanser formulation (ingredient list outlined in FIG. 8E) without 18B powder. FIG. 8E shows the ingredient list for an 18B powder cleanser, according to one embodiment of the invention.

FIG. 9A shows mean percent improvement of 2 wt % 18B powder solution for firmness and elasticity of the skin. *=p<0.05 of 2 wt % 18B powder in a basic skin cream at t=12 weeks compared to baseline measurement at t=0 weeks. FIG. 9B shows a graph of statistical improvement of 2 wt % 18B powder solution for lifting mid-face, elasticity, firmness, and overall skin healthy appearance over a period of 8 weeks. *=p<0.05 of 2 wt % 18B powder solution compared to empty vehicle. FIG. 9C shows a graph of skin results for a subjective panelist questionnaire after subjects used a 2 wt % 18B powder solution for 4 weeks. *=p<0.05 of 2 wt % 18B powder solution compared to empty vehicle.

FIG. 10 shows light microscopy images of a keratinocyte wound scratch model 48 hours after the scratch was made and a computer-generated quantification of the wound closure after incubating cells with and without 100 μg/mL of 18B powder.

FIG. 11A shows light microscopy images of a fibroblast wound scratch model 24 hours after the scratch was made and quantification of the wound closure after incubating cells with and without various concentrations of 18B powder (25 μg/mL and 50 μg/mL), as compared to a positive control. FIG. 11B shows a quantification of the percent coverage of the wounded area by migrating fibroblasts after incubating cells with and without various concentrations of 18B powder (25 μg/mL and 50 μg/mL), as compared to a positive control.

FIG. 12A shows additional light microscopy images of 18B powder resuspended in different solvents. FIG. 12B shows a comparison of powder diameters in various solvents as determined by image analysis. FIG. 12C shows a graphical comparison of powder diameters in various solvents versus cumulative percentage (%).

FIG. 13A shows quantification of the solubility of various recombinant 18B protein powder solutions as determined by size exclusion chromatography (SEC). FIG. 13B shows a table of the solubility results.

FIG. 14A shows histological cross-sections of ex-vivo tissues of untreated, empty vehicle, and 5% recombinant 18B protein samples at day 4 and day 8 timepoints. The dotted lined indicate the location of the original wounding site (left dotted line) and the extent of wound closure (right dotted line). FIG. 14B shows quantification results of average epidermal tongue length (μm) of samples at day 4 and day 8 timepoints. The data is plotted as average +/− standard deviation (**=p<0.01, *=p<0.05).

FIG. 15A shows histological cross-sections of ex-vivo tissues of untreated, empty vehicle, and 2% recombinant 18B protein samples with and without blue light irradiation stained for 8-OHdG. FIG. 15B shows quantification of the histology results, with and without blue light irradiation and plotted as average 8-OHdG stained surface %+/− standard deviation (**=p<0.01).

FIG. 16A shows histological cross-sections of ex-vivo tissues of untreated, empty vehicle, and 2% recombinant 18B protein samples with and without exposure to pollution and stained for Nrf2. FIG. 16B shows quantification results of Nrf2 expression with and without exposure to pollution. The data is plotted as average +/− standard deviation (**=p<0.01). FIG. 16C shows histological cross-sections of ex-vivo tissues of untreated, empty vehicle, and 2% recombinant 18B protein samples with and without exposure to pollution stained for IL-la. FIG. 16D shows quantification results of IL-la expression with and without exposure to pollution. The data is plotted as average +/− standard deviation (***=p<0.01).

FIG. 17A shows histological cross-sections of ex-vivo tissues of untreated, empty vehicle, and 5% recombinant 18B protein samples with and without exposure to UVB and stained with Mason's Trichrome to visualize cell viability. FIG. 17B shows quantification results the total number of sunburned cells with exposure to UVB. The data is plotted as average +/− standard deviation (*=p<0.05). FIG. 17C shows histological cross-sections of ex-vivo tissues of untreated, empty vehicle, and 5% recombinant 18B protein samples with and without exposure to UVB and stained for thymine dimers. FIG. 17D shows quantification results of thymine dimers expression with exposure to UVB. The data is plotted as average +/− standard deviation (**=p<0.01). FIG. 17E shows histological cross-sections of ex-vivo tissues of untreated, empty vehicle, and 5% recombinant 18B protein samples with and without exposure to UVA and stained for Nrf2. FIG. 17F shows quantification results of Nrf2 expression with exposure to UVA. The data is plotted as average +/− standard deviation (#=p<0.1).

FIG. 18 shows a mattifying effect of 18B powder on the skin when compared to an empty vehicle.

### DETAILED DESCRIPTION

The details of various embodiments of the invention are set forth in the description below. Other features, objects, and advantages of the invention will be apparent from the description. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

### Definitions

The following terms, unless otherwise indicated, shall be understood to have the following meanings:

The term “stability”, as used herein with respect to silk proteins, refers to the ability of the product not to form a gelation, discoloration or turbidity that is due to the self-aggregation of silk proteins. For example, U.S. Patent Publication No. 2015/0079012 (Wray et al.) is directed to the use of humectant, including glycerol to increase the shelf-stability of skincare products comprising full-length silk fibroin. U.S. Pat. No. 9,187,538 is directed to a skincare formulation comprising full-length silk fibroin that is shelf stable for up to 10 days. Both of these publications are incorporated herein by reference in their entirety.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally occurring and non-naturally occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

Throughout this specification and claims, the word “comprise” or variations such as “comprises” or “comprising,” will be understood to imply the inclusion of a stated integer or group of integers but not the exclusion of any other integer or group of integers.

The term “glass transition” as used herein refers to the transition of a substance or composition from a hard, rigid or “glassy” state into a more pliable, “rubbery” or “viscous” state.

The term “glass transition temperature” as used herein refers to the temperature at which a substance or composition undergoes a glass transition.

The term “melt transition” as used herein refers to the transition of a substance or composition from a rubbery state to a less-ordered liquid phase.

The term “melting temperature” as used herein refers to the temperature range over which a substance undergoes a melt transition.

The term “plasticizer” as used herein refers to any molecule that interacts with a polypeptide sequence to prevent the polypeptide sequence from forming tertiary structures and bonds and/or increases the mobility of the polypeptide sequence.

The term “powder” as used herein refers to a composition that is present in granular form, which may or may not be complexed or agglomerated with a solvent such as water or serum. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated. Dry powder may be produced by spray-drying, lyophilization, and/or according to methods known in the art.

The term “carrier” refers to a recombinant protein used for surface hydration, surface cleansing, surface defense, surface detoxification, surface exfoliation, surface improvement, coloring, and/or delivery of various additives or solvents, including, but not limited to, water, glycerin, alcohols, siloxane, oils, humectants, emollients, occlusive agents, active agents, and/or cosmetic adjuvants to a surface like skin, hair, or nails. The carrier as used herein comprises an outer shell and hollow core, e.g., 18B protein.

The term “cosmetics” as used herein includes make-up, foundation, skin care, hair care, and nail care products.

The term “make-up” as used herein refers to products that leave color on the face, including foundation, blacks and browns, i.e., mascara, concealers, eye liners, brow colors, eye shadows, blushers, lip colors, powders, solid emulsion compact, and so forth.

The term “foundation” as used herein refers to liquid, cream, mousse, pancake, compact, concealer or like product created or reintroduced by cosmetic companies to even out the overall coloring of the skin.

The term “skin care products” as used herein refer to those used to treat or care for, or somehow moisturize, improve, or clean the skin. Products contemplated by the phrase “skin care products” include, but are not limited to, creams, mists, serums, cleansing gels, ampules, adhesives, patches, bandages, toothpaste, anhydrous occlusive moisturizers, antiperspirants, deodorants, personal cleansing products, powder laundry detergent, fabric softener towels, occlusive drug delivery patches, nail polish, powders, tissues, wipes, hair conditioners-anhydrous, shaving creams, and the like.

The term “sagging” as used herein means the laxity, slackness, or the like condition of skin that occurs as a result of loss of, damage to, alterations to, and/or abnormalities in dermal elastin, muscle and/or subcutaneous fat.

The terms “treating” or “treatment” as used herein refer to the treatment (e.g., alleviation or elimination of symptoms and/or cure) and/or prevention or inhibition of the condition (e.g. a skin condition) or relief of symptoms.

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Recombinant Silk Proteins

The present disclosure describes embodiments of the invention including fibers synthesized from synthetic proteinaceous copolymers (i.e., recombinant polypeptides). Suitable proteinaceous co-polymers are discussed in U.S. Patent Publication No. 2016/0222174, published Aug. 45, 2016, U.S. Patent Publication No. 2018/0111970, published Apr. 26, 2018, and U.S. Patent Publication No. 2018/0057548, published Mar. 1, 2018, each of which are incorporated by reference herein in its entirety.

In some embodiments, the synthetic proteinaceous copolymers are made from silk-like polypeptide sequences. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful molded body compositions by secretion from an industrially scalable microorganism. Large (approximately 40 kDa to approximately 100 kDa) block copolymer polypeptides engineered from silk repeat domain fragments, including sequences from almost all published amino acid sequences of silk polypeptides, can be expressed in the modified microorganisms described herein. In some embodiments, silk polypeptide sequences are matched and designed to produce highly expressed and secreted polypeptides capable of molded body formation.

In some embodiments, block copolymers are engineered from a combinatorial mix of silk polypeptide domains across the silk polypeptide sequence space. In some embodiments, the block copolymers are made by expressing and secreting in scalable organisms (e.g., yeast, fungi, and gram positive bacteria). In some embodiments, the block copolymer polypeptide comprises 0 or more N-terminal domains (NTD), 1 or more repeat domains (REP), and 0 or more C-terminal domains (CTD). In some aspects of the embodiment, the block copolymer polypeptide is >100 amino acids of a single polypeptide chain. In some embodiments, the block copolymer polypeptide comprises a domain that is at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of a block copolymer polypeptide as disclosed in International Publication No. WO/2015/042164, “Methods and Compositions for Synthesizing Improved Silk Fibers,” incorporated by reference in its entirety.

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011). For example:

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block copolymer polypeptides having sequence similarity and/or amino acid composition similarity to the repeat domains of native silk proteins can be used to manufacture on commercial scales consistent molded bodies that have properties that recapitulate the properties of corresponding molded bodies made from natural silk polypeptides.

In some embodiments, a list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for expression in Pichia (Komagataella) pastoris. The DNA sequences are each cloned into an expression vector and transformed into Pichia (Komagataella) pastoris. In some embodiments, various silk domains demonstrating successful expression and secretion are subsequently assembled in combinatorial fashion to build silk molecules capable of molded body formation.

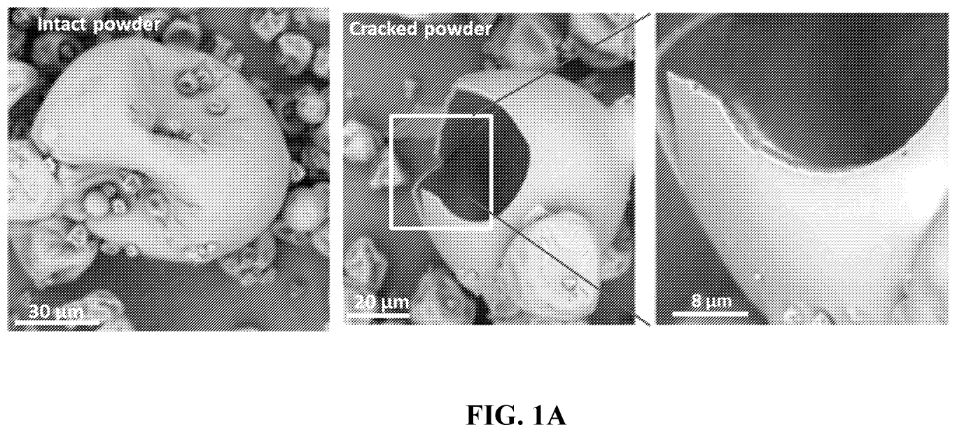

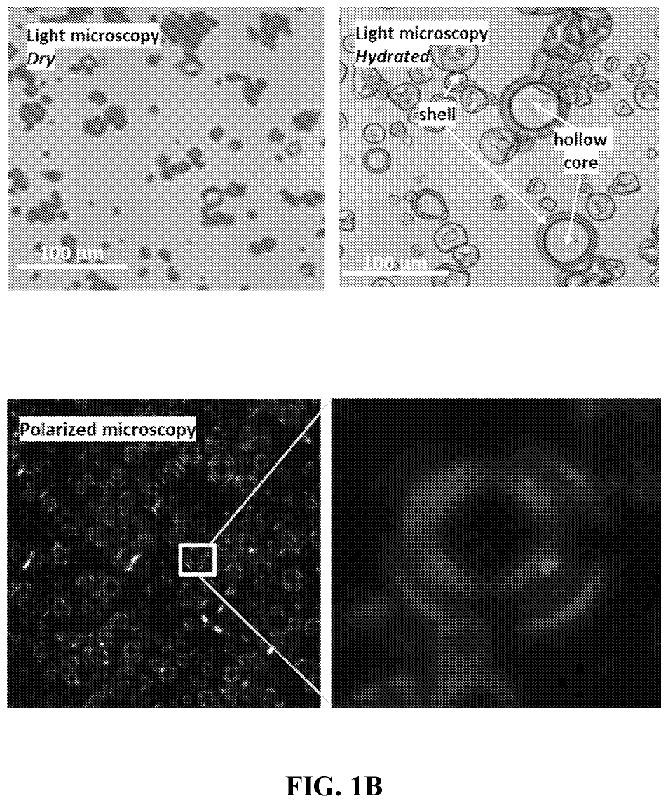

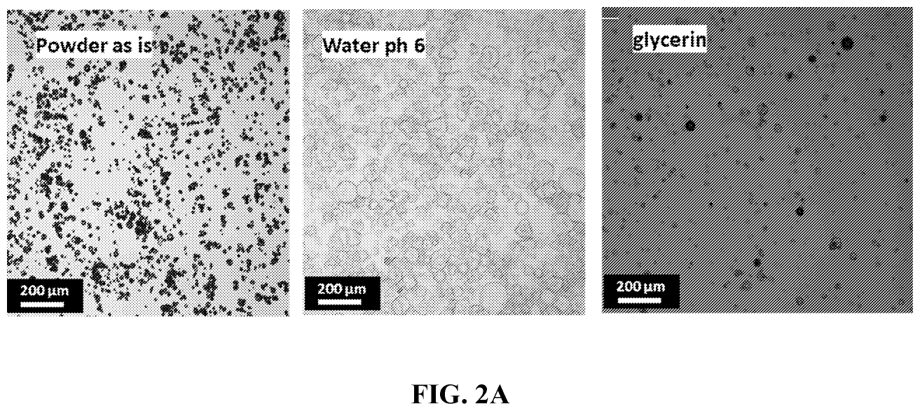

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture, as depicted in FIG. 1. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1A lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. In some embodiments, block sequences comprise a glycine rich region followed by a polyA region. In some embodiments, short (˜1-10) amino acid motifs appear multiple times inside of blocks. For the purpose of this invention, blocks from different natural silk polypeptides can be selected without reference to circular permutation (i.e., identified blocks that are otherwise similar between silk polypeptides may not align due to circular permutation). Thus, for example, a “block” of SGAGG (SEQ ID NO: 494) is, for the purposes of the present invention, the same as GSGAG (SEQ ID NO: 495) and the same as GGSGA (SEQ ID NO: 496); they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 1A
Samples of Block Sequences

| Species | Silk Type | Representative Block Amino Acid Sequence |
|---|---|---|
| Aliatypus | Fibroin 1 | GAASSSSTIITTKSASASAAADASAAATASAASRSSANAAASAF |
| gulosus | | AQSFSSILLESGYFCSIFGSSISSSYAAAIASAASRAAAESNGY |
| | | TTHAYACAKAVASAVERVTSGADAYAYAQAISDALSHALLYTGR |
| | | LNTANANSLASAFAYAFANAAAQASASSASAGAASASGAASASG |
| | | AGSAS |
| Plectreurys | Fibroin 1 | GAGAGAGAGAGAGAGAGSGASTSVSTSSSSGSGAGAGAGSGAGS |
| tristis | | GAGAGSGAGAGAGAGGAGAGFGSGLGLGYGVGLSSAQAQAQAQA |
| | | AAQAQAQAQAQAYAAAQAQAQAQAQAQAAAAAAAAAAA |
| Plectreurys | Fibroin 4 | GAAQKQPSGESSVATASAAATSVTSGGAPVGKPGVPAPIFYPQG |
| tristis | | PLQQGPAPGPSNVQPGTSQQGPIGGVGGSNAFSSSFASALSLNR |
| | | GFTEVISSASATAVASAFQKGLAPYGTAFALSAASAAADAYNSI |
| | | GSGANAFAYAQAFARVLYPLVQQYGLSSSAKASAFASAIASSES |
| | | SGTSGQGPSIGQQQPPVTISAASASAGASAAAVGGGQVGQGPYG |
| | | GQQQSTAASASAAAATATS |
| Araneus | TuSp | GNVGYQLGLKVANSLGLGNAQALASSLSQAVSAVGVGASSNAYA |
| gemmoides | | NAVSNAVGQVLAGQGILNAANAGSLASSFASALSSSAASVASQS |
| | | ASQSQAASQSQAAASAFRQAASQSASQSDSRAGSQSSTKTTSTS |
| | | TSGSQADSRSASSSASQASASAFAQQSSASLSSSSSFSSAFSSA |
| | | TSISAV |
| Argiope | TuSp | GSLASSFASALSASAASVASSAAAQAASQSQAAASAFSRAASQS |
| aurantia | | ASQSAARSGAQSISTTTTTSTAGSQAASQSASSAASQASASSFA |
| | | RASSASLAASSSESSAFSSANSLSALGNVGYQLGENVANNLGIG |
| | | NAAGLGNALSQAVSSVGVGASSSTYANAVSNAVGQFLAGQGILN |
| | | AANA |
| Deinopis | TuSp | GASASAYASAISNAVGPYLYGLGLENQANAASFASSFASAVSSA |
| spinosa | | VASASASAASSAYAQSAAAQAQAASSAFSQAAAQSAAAASAGAS |
| | | AGAGASAGAGAVAGAGAVAGAGAVAGASAAAASQAAASSSASAV |
| | | ASAFAQSASYALASSSAFANAFASATSAGYLGSLAYQLGLTTAY |
| | | NLGLSNAQAFASTLSQAVTGVGL |
| Nephila | TuSp | GATAASYGNALSTAAAQFFATAGLLNAGNASALASSFARAFSAS |
| clavipes | | AESQSFAQSQAFQQASAFQQAASRSASQSAAEAGSTSSSTTTTT |
| | | SAARSQAASQSASSSYSSAFAQAASSSLATSSALSRAFSSVSSA |
| | | SAASSLAYSIGLSAARSLGIADAAGLAGVLARAAGALGQ |
| Argiope | Flag | GGAPGGGPGGAGPGGAGFGPGGGAGFGPGGGAGFGPGGAAGGPG |
| trifasciata | | GPGGPGGPGGAGGYGPGGAGGYGPGGVGPGGAGGYGPGGAGGYG |
| | | PGGSGPGGAGPGGAGGEGPVTVDVDVTVGPEGVGGGPGGAGPGG |
| | | AGFGPGGGAGFGPGGAPGAPGGPGGPGGPGGPGGPGGVGPGGAG |
| | | GYGPGGAGGVGPAGTGGFGPGGAGGFGPGGAGGFGPGGAGGFGP |
| | | AGAGGYGPGGVGPGGAGGFGPGGVGPGGSGPGGAGGEGPVTVDV |
| | | DVSV |
| Nephila | Flag | GVSYGPGGAGGPYGPGGPYGPGGEGPGGAGGPYGPGGVGPGGSG |
| clavipes | | PGGYGPGGAGPGGYGPGGSGPGGYGPGGSGPGGYGPGGSGPGGY |
| | | GPGGSGPGGYGPGGYGPGGSGPGGSGPGGSGPGGYGPGGTGPGG |
| | | SGPGGYGPGGSGPGGSGPGGYGPGGSGPGGFGPGGSGPGGYGPG |
| | | GSGPGGAGPGGVGPGGFGPGGAGPGGAAPGGAGPGGAGPGGAGP |
| | | GGAGPGGAGPGGAGPGGAGGAGGAGGSGGAGGSGGTTIIEDLDI |
| | | TIDGADGPITISEELPISGAGGSGPGGAGPGGVGPGGSGPGGVG |
| | | PGGSGPGGVGPGGSGPGGVGPGGAGGPYGPGGSGPGGAGGAGGP |
| | | GGAYGPGGSYGPGGSGGPGGAGGPYGPGGEGPGGAGGPYGPGGA |
| | | GGPYGPGGAGGPYGPGGEGGPYGP |
| Latrodectus | AcSp | GINVDSDIGSVTSLILSGSTLQMTIPAGGDDLSGGYPGGFPAGA |
| hesperus | | QPSGGAPVDFGGPSAGGDVAAKLARSLASTLASSGVFRAAFNSR |
| | | VSTPVAVQLTDALVQKIASNLGLDYATASKLRKASQAVSKVRMG |
| | | SDTNAYALAISSALAEVLSSSGKVADANINQIAPQLASGIVLGV |
| | | STTAPQFGVDLSSINVNLDISNVARNMQASIQGGPAPITAEGPD |
| | | FGAGYPGGAPTDLSGLDMGAPSDGSRGGDATAKLLQALVPALLK |
| | | SDVFRAIYKRGTRKQVVQYVTNSALQQAASSLGLDASTISQLQT |
| | | KATQALSSVSADSDSTAYAKAFGLAIAQVLGTSGQVNDANVNQI |
| | | GAKLATGILRGSSAVAPRLGIDLS |
| Argiope | AcSp | GAGYTGPSGPSTGPSGYPGPLGGGAPFGQSGFGGSAGPQGGFGA |
| trifasciata | | TGGASAGLISRVANALANTSTLRTVLRTGVSQQIASSVVQRAAQ |
| | | SLASTLGVDGNNLARFAVQAVSRLPAGSDTSAYAQAFSSALENA |
| | | GVLNASNIDTLGSRVLSALLNGVSSAAQGLGINVDSGSVQSDIS |
| | | SSSSFLSTSSSSASYSQASASSTS |
| Uloborus | AcSp | GASAADIATAIAASVATSLQSNGVLTASNVSQLSNQLASYVSSG |
| diversus | | LSSTASSLGIQLGASLGAGFGASAGLSASTDISSSVEATSASTL |
| | | SSSASSTSVVSSINAQLVPALAQTAVLNAAFSNINTQNAIRIAE |
| | | LLTQQVGRQYGLSGSDVATASSQIRSALYSVQQGSASSAYVSAI |
| | | VGPLITALSSRGVVNASNSSQIASSLATAILQFTANVAPQFGIS |
| | | IPTSAVQSDLSTISQSLTAISSQTSSSVDSSTSAFGGISGPSGP |
| | | SPYGPQPSGPTFGPGPSLSGLTGFTATFASSFKSTLASSTQFQL |
| | | IAQSNLDVQTRSSLISKVLINALSSLGISASVASSIAASSSQSL |
| | | LSVSA |
| Euprosthenops | MaSp1 | GGQGGQGQGRYGQGAGSSAAAAAAAAAAAAAA |
| australis | | |
| Tetragnatha | MaSp1 | GGLGGGQGAGQGGQQGAGQGGYGSGLGGAGQGASAAAAAAAA |
| kauaiensis | | |
| Argiope | MaSp2 | GGYGPGAGQQGPGSQGPGSGGQQGPGGLGPYGPSAAAAAAAA |
| aurantia | | |
| Deinopis | MaSp2 | GPGGYGGPGQQGPGQGQYGPGTGQQGQGPSGQQGPAGAAAAAAA |
| spinosa | | AA |
| Nephila | MaSp2 | GPGGYGLGQQGPGQQGPGQQGPAGYGPSGLSGPGGAAAAAAA |
| clavata | | |
| Deinopis | MiSp | GAGYGAGAGAGGGAGAGTGYGGGAGYGTGSGAGYGAGVGYGAGA |
| Spinosa | | GAGGGAGAGAGGGTGAGAGGGAGAGYGAGTGYGAGAGAGGGAGA |
| | | GAGAGAGAGAGAGSGAGAGYGAGAGYGAGAGAGGVAGAGAAGGA |
| | | GAAGGAGAAGGAGAAGGAGAGAGAGSGAGAGAGGGARAGAGG |
| | | [SEQ ID NO: 1115] |
| Latrodectus | MiSp | GGGYGRGQGAGAGVGAGAGAAAGAAAIARAGGYGQGAGGYGQGQ |
| hesperus | | GAGAAAGAAAGAGAGGYGQGAGGYGRGQGAGAGAGAGAGARGYG |
| | | QGAGAGAAAGAAASAGAGGYGQGAGGYGQGQGAGAAAGAAASAG |
| | | AGGYGQGAGGYGQGQGA [SEQ ID NO: 1226] |
| Nephila | MiSp | GAGAGGAGYGRGAGAGAGAAAGAGAGAAAGAGAGAGGYGGQGGY |
| clavipes | | GAGAGAGAAAAAGAGAGGAAGYSRGGRAGAAGAGAGAAAGAGAG |
| | | AGGYGGQGGYGAGAGAGAAAAAGAGSGGAGGYGRGAGAGAAAGA |
| | | GAAAGAGAGAGGYGGQGGYGAGAGAAAAA [SEQ ID NO: |
| | | 1234] |
| Nephilengys | MiSp | GAGAGVGGAGGYGSGAGAGAGAGAGAASGAAAGAAAGAGAGGAG |
| cruentata | | GYGTGQGYGAGAGAGAGAGAGGAGGYGRGAGAGAGAGAGGAGGY |
| | | GAGQGYGAGAGAGAAAAAGDGAGAGGAGGYGRGAGAGAGAGAAA |
| | | GAGAGGAGGYGAGQGYGAGAGAGAAAGAGAGGAGGYGAGQGYGA |
| | | GAGAGAAAAA [SEQ ID NO: 1239] |
| Uloborus | MiSp | GSGAGAGSGYGAGAGAGAGSGYGAGSSASAGSAINTQTVTSSTT |
| diversus | | TSSQSSAAATGAGYGTGAGTGASAGAAASGAGAGYGGQAGYGQG |
| | | AGASARAAGSGYGAGAGAAAAAGSGYGAGAGAGAGSGYGAGAAA |
| | | [SEQ ID NO: 1246] |
| Uloborus | MiSp | GAGAGYRGQAGYIQGAGASAGAAAAGAGVGYGGQAGYGQGAGAS |
| diversus | | AGAAAAAGAGAGRQAGYGQGAGASAGAAAAGAGAGRQAGYGQGA |
| | | GASAGAAAAGADAGYGGQAGYGQGAGASAGAAASGAGAGYGGQA |
| | | GYGQGAGASAGAAAAGAGAGYLGQAGYGQGAGASAGAAAGAGAG |
| | | YGGQAGYGQGTGAAASAAASSA [SEQ ID NO: 1249] |
| Araneus | MaSp1 | GGQGGQGGYGGLGSQGAGQGGYGAGQGAAAAAAAAGGAGGAGRG |
| ventricosus | | GLGAGGAGQGYGAGLGGQGGAGQAAAAAAAGGAGGARQGGLGAG |
| | | GAGQGYGAGLGGQGGAGQGGAAAAAAAAGGQGGQGGYGGLGSQG |
| | | AGQGGYGAGQGGAAAAAAAAGGQGGQGGYGGLGSQGAGQGGYGG |
| | | RQGGAGAAAAAAAA [SEQ ID NO: 1312] |
| Dolomedes | MaSp1 | GGAGAGQGSYGGQGGYGQGGAGAATATAAAAGGAGSGQGGYGGQ |
| tenebrosus | | GGLGGYGQGAGAGAAAAAAAAAGGAGAGQGGYGGQGGQGGYGQG |
| | | AGAGAAAAAAGGAGAGQGGYGGQGGYGQGGGAGAAAAAAAASGG |
| | | SGSGQGGYGGQGGLGGYGQGAGAGAGAAASAAAA [SEQ ID |
| | | NO: 1345] |
| Nephilengys | MaSp | GGAGQGGYGGLGGQGAGAAAAAAGGAGQGGYGGQGAGQGAAAAA |
| cruentata | | ASGAGQGGYEGPGAGQGAGAAAAAAGGAGQGGYGGLGGQGAGQG |
| | | AGAAAAAAGGAGQGGYGGLGGQGAGQGAGAAAAAAGGAGQGGYG |
| | | GQGAGQGAAAAAAGGAGQGGYGGLGSGQGGYGRQGAGAAAAAAA |
| | | A [SEQ ID NO: 1382] |
| Nephilengys | MaSp | GGAGQGGYGGLGGQGAGAAAAAAGGAGQGGYGGQGAGQGAAAAA |
| cruentata | | ASGAGQGGYGGPGAGQGAGAAAAAAGGAGQGGYGGLGGQGAGQG |
| | | AGAAAAAAGGAGQGGYGGQGAGQGAAAAAAGGAGQGGYGGLGSG |
| | | QGGYGGQGAGAAAAAGGAGQGGYGGLGGQGAGQGAGAAAAAA |
| | | [SEQ ID NO: 1383] |

Fiber-forming block copolymer polypeptides from the blocks and/or macro-repeat domains, according to certain embodiments of the invention, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers or molded bodies include natural amino acid sequence information and other modifications described herein. The repeat domain is decomposed into repeat sequences containing representative blocks, usually 1-8 depending upon the type of silk, that capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macrorepeats.

In some embodiments, non-repetitive N- and C-terminal domains can be selected for synthesis. In some embodiments, N-terminal domains can be by removal of the leading signal sequence, e.g., as identified by SignalP (Peterson, T. N., et. Al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3× FLAG nucleotide coding sequence. In some embodiments, the silk polypeptide nucleotide coding sequence is operatively linked to other affinity tags such as 6-8 His residues.

In some embodiments, the recombinant silk polypeptides are based on recombinant spider silk protein fragment sequences derived from MaSp2, such as from the species Argiope bruennichi. In some embodiments, the synthesized fiber contains protein molecules that include two to twenty repeat units, in which a molecular weight of each repeat unit is greater than about 20 kDa. Within each repeat unit of the copolymer are more than about 60 amino acid residues, often in the range 60 to 100 amino acids that are organized into a number of “quasi-repeat units.” In some embodiments, the repeat unit of a polypeptide described in this disclosure has at least 95% sequence identity to a MaSp2 dragline silk protein sequence.

The repeat unit of the proteinaceous block copolymer that forms fibers with good mechanical properties can be synthesized using a portion of a silk polypeptide. These polypeptide repeat units contain alanine-rich regions and glycine-rich regions, and are 150 amino acids in length or longer. Some exemplary sequences that can be used as repeats in the proteinaceous block copolymers of this disclosure are provided in in co-owned PCT Publication WO 2015/042164, incorporated by reference in its entirety, and were demonstrated to express using a Pichia expression system.

In some embodiments, the silk protein comprises: at least two occurrences of a repeat unit, the repeat unit comprising: more than 150 amino acid residues and having a molecular weight of at least 10 kDa; an alanine-rich region with 6 or more consecutive amino acids, comprising an alanine content of at least 80%; a glycine-rich region with 12 or more consecutive amino acids, comprising a glycine content of at least 40% and an alanine content of less than 30%; and wherein the fiber comprises at least one property selected from the group consisting of a modulus of elasticity greater than 550 cN/tex, an extensibility of at least 10% and an ultimate tensile strength of at least 15 cN/tex.

In some embodiments, wherein the recombinant silk protein comprises repeat units wherein each repeat unit has at least 95% sequence identity to a sequence that comprises from 2 to 20 quasi-repeat units; each quasi-repeat unit comprises {GGY-[GPG-X₁]n₁-GPS-(A)ₙ₂}, wherein for each quasi-repeat unit; X₁ is independently selected from the group consisting of SGGQQ, GAGQQ, GQGOPY, AGQQ, and SQ; and n1 is from 4 to 8, and n2 is from 6-10. The repeat unit is composed of multiple quasi-repeat units.

In some embodiments, 3 “long” quasi repeats are followed by 3 “short” quasi-repeat units. As mentioned above, short quasi-repeat units are those in which n1=4 or 5. Long quasi-repeat units are defined as those in which n1=6, 7 or 8. In some embodiments, all of the short quasi-repeats have the same X₁ motifs in the same positions within each quasi-repeat unit of a repeat unit. In some embodiments, no more than 3 quasi-repeat units out of 6 share the same X₁ motifs.

In additional embodiments, a repeat unit is composed of quasi-repeat units that do not use the same X₁ more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X₁ more than 2 times in a single quasi-repeat unit of the repeat unit.

In some embodiments, the recombinant silk polypeptide comprises the polypeptide sequence of SEQ ID NO: 1 (i.e., 18B). In some embodiments, the repeat unit is a polypeptide comprising SEQ ID NO: 2. These sequences are provided in Table 1B:

TABLE 1B
Exemplary polypeptides sequences of recombinant
protein and repeat unit

| SEQ | |
|---|---|
| ID | Polypeptide Sequence |
| SEQ | GGYGPGAGQQGPGSGGQQGPGGQGPYGSGQQGPGGAGQQGPGGQGPYGPGAAAAAAAAAG |
| ID | GYGPGAGQQGPGGAGQQGPGSQGPGGQGPYGPGAGQQGPGSQGPGSGGQQGPGGQGPYGP |
| NO: | SAAAAAAAAAGGYGPGAGQRSQGPGGQGPYGPGAGQQGPGSQGPGSGGQQGPGGQGPYGP |
| 1 | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAAGGYGPGAGQQGPGSGGQQGPGGQGPYGSGQQGPGGAGQQGPGGQG |
| | PYGPGAAAAAAAAAGGYGPGAGQQGPGGAGQQGPGSQGPGGQGPYGPGAGQQGPGSQGPG |
| | SGGQQGPGGQGPYGPSAAAAAAAAAGGYGPGAGQRSQGPGGQGPYGPGAGQQGPGSQGPG |
| | SGGQQGPGGQGPYGPSAAAAAAAAGGYGPGAGQQGPGSQGPGSGGQQGPGGQGPYGPGAA |
| | AAAAAVGGYGPGAGQQGPGSQGPGSGGQQGPGGQGPYGPSAAAAAAAAGGYGPGAGQQGP |
| | GSQGPGSGGQQGPGGQGPYGPSAAAAAAAAGGYGPGAGQQGPGSGGQQGPGGQGPYGSGQ |
| | QGPGGAGQQGPGGQGPYGPGAAAAAAAAAGGYGPGAGQQGPGGAGQQGPGSQGPGGQGPY |
| | GPGAGQQGPGSQGPGSGGQQGPGGQGPYGPSAAAAAAAAAGGYGPGAGQRSQGPGGQGPY |
| | GPGAGQQGPGSQGPGSGGQQGPGGQGPYGPSAAAAAAAAGGYGPGAGQQGPGSQGPGSGG |
| | QQGPGGQGPYGPGAAAAAAAVGGYGPGAGQQGPGSQGPGSGGQQGPGGQGPYGPSAAAAA |
| | AAAGGYGPGAGQQGPGSQGPGSGGQQGPGGQGPYGPSAAAAAAAA |
| SEQ | GGYGPGAGQQGPGSGGQQGPGGQGPYGSGQQGPGGAGQQGPGGQGPYGPGAAAAAAAAAG |
| ID | GYGPGAGQQGPGGAGQQGPGSQGPGGQGPYGPGAGQQGPGSQGPGSGGQQGPGGQGPYGP |
| NO: | SAAAAAAAAAGGYGPGAGQRSQGPGGQGPYGPGAGQQGPGSQGPGSGGQQGPGGQGPYGP |
| 2 | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAA |

In some embodiments, the structure of fibers formed from the described recombinant silk polypeptides form beta-sheet structures, beta-turn structures, or alpha-helix structures. In some embodiments, the secondary, tertiary and quaternary protein structures of the formed fibers are described as having nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix. Without intending to be limited by theory, the structural properties of the proteins within the spider silk are theorized to be related to fiber mechanical properties. Crystalline regions in a fiber have been linked with the tensile strength of a fiber, while the amorphous regions have been linked to the extensibility of a fiber. The major ampullate (MA) silks tend to have higher strengths and less extensibility than the flagelliform silks, and likewise the MA silks have higher volume fraction of crystalline regions compared with flagelliform silks. Furthermore, theoretical models based on the molecular dynamics of crystalline and amorphous regions of spider silk proteins, support the assertion that the crystalline regions have been linked with the tensile strength of a fiber, while the amorphous regions have been linked to the extensibility of a fiber. Additionally, the theoretical modeling supports the importance of the secondary, tertiary and quaternary structure on the mechanical properties of RPFs. For instance, both the assembly of nano-crystal domains in a random, parallel and serial spatial distributions, and the strength of the interaction forces between entangled chains within the amorphous regions, and between the amorphous regions and the nano-crystalline regions, influenced the theoretical mechanical properties of the resulting fibers.

In some embodiments, the molecular weight of the silk protein may range from 20 kDa to 2000 kDa, or greater than 20 kDa, or greater than 10 kDa, or greater than 5 kDa, or from 5 to 400 kDa, or from 5 to 300 kDa, or from 5 to 200 kDa, or from 5 to 100 kDa, or from 5 to 50 kDa, or from 5 to 500 kDa, or from 5 to 1000 kDa, or from 5 to 2000 kDa, or from 10 to 400 kDa, or from 10 to 300 kDa, or from 10 to 200 kDa, or from 10 to 100 kDa, or from 10 to 50 kDa, or from 10 to 500 kDa, or from 10 to 1000 kDa, or from 10 to 2000 kDa, or from 20 to 400 kDa, or from 20 to 300 kDa, or from 20 to 200 kDa, or from 40 to 300 kDa, or from 40 to 500 kDa, or from 20 to 100 kDa, or from 20 to 50 kDa, or from 20 to 500 kDa, or from 20 to 1000 kDa, or from 20 to 2000 kDa.

### Characterization of Recombinant Spider Silk Polypeptide Powder Impurities and Degradation

Different recombinant spider silk polypeptides have different physiochemical properties such as melting temperature and glass transition temperature based on the strength and stability of the secondary and tertiary structures formed by the proteins. Silk polypeptides form beta sheet structures in a monomeric form. In the presence of other monomers, the silk polypeptides form a three-dimensional crystalline lattice of beta sheet structures. The beta sheet structures are separated from, and interspersed with, amorphous regions of polypeptide sequences.

Beta sheet structures are extremely stable at high temperatures—the melting temperature of beta-sheets is approximately 257° C. as measured by fast scanning calorimetry. See Cebe et al., Beating the Heat—Fast Scanning Melts Silk Beta Sheet Crystals, Nature Scientific Reports 3:1130 (2013). As beta sheet structures are thought to stay intact above the glass transition temperature of silk polypeptides, it has been postulated that the structural transitions seen at the glass transition temperature of recombinant silk polypeptides are due to increased mobility of the amorphous regions between the beta sheets.

Plasticizers lower the glass transition temperature and the melting temperature of silk proteins by increasing the mobility of the amorphous regions and potentially disrupting beta sheet formation. Suitable plasticizers used for this purpose include, but are not limited to, water and polyalcohols (polyols) such as glycerol, triglycerol, hexaglycerol, and decaglycerol. Other suitable plasticizers include, but are not limited to, Dimethyl Isosorbite; adipic acid; amide of dimethylaminopropyl amine and caprylic/capric acid; acetamide and any combination thereof.

As hydrophilic portions of silk polypeptides can bind ambient water present in the air as humidity, water will almost always be present, the bound ambient water may plasticize silk polypeptides. In some embodiments, a suitable plasticizer may be glycerol, present either alone or in combination with water or other plasticizers. Other suitable plasticizers are discussed above.

In addition, in instances where recombinant silk polypeptides are produced by fermentation and recovered as recombinant silk polypeptide powder from the same, there may be impurities present in the recombinant silk polypeptide powder that act as plasticizers or otherwise inhibit the formation of tertiary structures. For example, residual lipids and sugars may act as plasticizers and thus influence the glass transition temperature of the protein by interfering with the formation of tertiary structures.

Various well-established methods may be used to assess the purity and relative composition of recombinant silk polypeptide powder or composition. Size Exclusion Chromatography separates molecules based on their relative size and can be used to analyze the relative amounts of recombinant silk polypeptide in its full-length polymeric and monomeric forms as well as the amount of high, low and intermediate molecular weight impurities in the recombinant silk polypeptide powder. Similarly, Rapid High Performance Liquid Chromatography may be used to measure various compounds present in a solution such as monomeric forms of the recombinant silk polypeptide. Ion Exchange Liquid Chromatography may be used to assess the concentrations of various trace molecules in solution, including impurities such as lipids and sugars. Other methods of chromatography and quantification of various molecules such as mass spectrometry are well established in the art.

Depending on the embodiment, the recombinant silk polypeptide may have a purity calculated based on the amount of the recombinant silk polypeptide in is monomeric form by weight relative to the other components of the recombinant silk polypeptide powder. In various instances, the purity can range from 50% by weight to 90% by weight, depending on the type of recombinant silk polypeptide and the techniques used to recover, separate and post-process the recombinant silk polypeptide powder.

Both Size Exclusion Chromatography and Reverse Phase High Performance Liquid Chromatography are useful in measuring full-length recombinant silk polypeptide, which makes them useful techniques for determining whether processing steps have degraded the recombinant silk polypeptide by comparing the amount of full-length silk polypeptide in a composition before and after processing. In various embodiments of the present invention, the amount of full-length recombinant silk polypeptide present in a composition before and after processing may be subject to minimal degradation. The amount of degradation may be in the range 0.001% by weight to 10% by weight, or 0.01% by weight to 6% by weight, e.g. less than 10% or 8% or 6% by weight, or less than 5% by weight, less than 3% by weight or less than 1% by weight.

### Recombinant Silk Compositions

Depending on the embodiment, suitable concentrations of recombinant silk polypeptide powder by weight in the recombinant silk composition ranges from: 1 to 25% by weight, 1 to 30% by weight, to 70% by weight, 10 to 60% by weight, 15 to 50% by weight, 18 to 45% by weight, or 20 to 41% by weight.

Without intending to be limited by theory, in various embodiments of the present invention, inducing the recombinant silk composition may be used in applications where it is desirable to prevent the aggregation of the monomeric recombinant silk polypeptide into its crystalline polymeric form or to control the transition of the recombinant silk polypeptide into its crystalline polymeric form at a later stage in processing. In other embodiments, such inducing is not required.

In one specific embodiment, the recombinant silk composition may be used to prevent aggregation of the recombinant silk polypeptide prior to blending the recombinant silk polypeptide with a second polymer. In another specific embodiment, the recombinant silk composition may be used to create a base for a cosmetic or skincare product where the recombinant silk polypeptide is present in the base in its monomeric form. In this embodiment, having the recombinant silk polypeptide in its monomeric form in a base allows for the controlled aggregation of the monomer into its crystalline polymeric form upon contact with skin or through various other chemical reactions.

In various embodiments, the temperature to which the recombinant silk composition is heated will be minimized in order to minimize or entirely prevent degradation of the recombinant silk polypeptide. In specific embodiments, the recombinant silk melt will be heated to a temperature of less than 120° C., less than 100° C., less than 80° C., less than 60° C., less than 40° C., or less than 20° C. Often the melt will be at a temperature in the range 10° C. to 120° C., 10° C. to 100° C., 15° C. to 80° C., 15° C. to 60° C., 18° C. to 40° C. or 18° C. to 22° C. during processing. In other embodiments, the recombinant silk composition is not heated. In such embodiments, the presence of heat is not required to form a recombinant silk composition.

The amount of degradation of the recombinant silk polypeptide may be measured using various techniques. As discussed above, the amount of degradation of the recombinant silk polypeptide may be measured using Size Exclusion Chromatography to measure the amount of full-length recombinant silk polypeptide present. In various embodiments, the composition is degraded in an amount of less than 6.0 weight % after it is formed into a molded body. In another embodiment, the composition is degraded in an amount of less than 4.0 weight % after molding, less than 3.0 weight %, less than 2.0 weight %, or less than 1.0 weight %, such that the amount of degradation may be in the range 0.001% by weight to 10%, 8%, 6%, 4%, 3%, 2% or 1% by weight, or 0.01% by weight to 6%, 4%, 3%, 2% or 1% by weight. In another embodiment, the recombinant silk protein in the composition is substantially non-degraded. In a similar embodiment, the recombinant silk protein in the composition is substantially non-degraded over a period of time, at least 1 day, 1 month, 1 year, or 5 years.

In some embodiments, the recombinant silk composition is physically stable. In various embodiments, the compositions remain in its material form, e.g., a powder, for a prolonged period of time, with a prolonged shelf life. On prolonged use, the recombinant silk composition remains substantially stable.

In most embodiments of the present invention, the recombinant silk composition is a powder. In some embodiments, the recombinant silk composition is spray-dried. In other embodiments, the recombinant silk composition is freeze-dried or vacuum-dried. The terms “spray-drying” and “spray-dried” are used herein for simplicity but the skilled person will appreciate that freeze-drying or lyophilization and vacuum drying can be substituted for spray-drying as appropriate. These compositions may be stored dry.

The 18B protein is more stable in a dried form than in an aqueous slurry. In some embodiments, spray-dried recombinant silk is obtained as follows: a slurry composition comprising extracted recombinant silk is kept chilled during the drying step. It is pumped to a tall form spray dryer where the moisture content of the resulting powder is tightly controlled. As the protein powder is hydroscopic, the final powder collection and packout is performed to minimize reintroduction of moisture. The design of the packaging material should minimize moisture and light exposure.

In some embodiments, recovery and separation of the recombinant silk polypeptide from a cell culture is performed as follows: i) extraction and separation, ii) urea removal by ultrafiltration, iii) washing by precipitation, iv) salt removal and protein concentration, and v) spray drying.

In some embodiments, to freeze-dry a composition it is cooled until it solidifies and placed under reduced pressure to cause the most volatile ingredients in the composition to sublime. The solid residue may form a single mass which requires milling to form a fine powder. A typical freeze-dried powder comprises porous irregular shaped particles and readily hydrates. As freeze-drying does not require strong heat it is used to produce powders which comprise volatile ingredients. In some embodiments, the recombinant silk composition is deep freeze-dried at a temperature below about −100° C.

After formation of the recombinant silk composition, the crystallinity of the recombinant silk composition can increase, thereby strengthening the composition. In some embodiments, the recombinant silk composition stays the same or decreases. In some embodiments, the crystallinity index of the recombinant silk composition as measured by X-ray crystallography is from 2% to 90%. In some other embodiments, the crystallinity index of the recombinant silk composition as measured by X-ray crystallography is at least 3%, at least 4%, at least 5%, at least 6%, or at least 7%.

In some embodiments of the present invention, the recombinant silk composition is a solid or film. In some embodiments, the recombinant silk composition is a powder. In some embodiments, the solid or film will be substantially homogeneous meaning that the material, as inspected by light microscopy, has a low amount or does not have any inclusions or precipitates. In some embodiments, light microscopy may be used to measure birefringence which can be used as a proxy for alignment of the recombinant silk into a three-dimensional lattice. Birefringence is the optical property of a material having a refractive index that depends on the polarization and propagation of light. Specifically, a high degree of axial order as measured by birefringence can be linked to high tensile strength. In some embodiments, recombinant silk solids and films will have minimal birefringence. In various embodiments, the solid is a bead. In some other embodiments, the solid functions as an exfoliant. The recombinant silk solid may be in the form of a gentle skin scrub for the skin. In some embodiments, the material form is a roll, pellet, sheet, or flake.

In some embodiments, the recombinant silk protein comprises a hollow core and/or a shell. In some embodiments, the recombinant silk protein ranges from about 1 μm to about 30 μm in diameter, about 5 μm to about 20 μm, or about 10 μm to about 50 μm in diameter, while recombinant silk protein in water ranges from about 20 to about 80 μm in diameter, about 30 μm to about 70 μm, or about 40 μm to about 100 μm in diameter.

### Solvents

In some embodiments, the silk polypeptide may be subjected to one or more solvents. In such embodiments, the hollow core contains the solvent such as liquid water or glycerin, either in form of liquid water itself, or as a liquid aqueous solution, an emulsion containing liquid water or as an aqueous dispersion. In certain embodiments, the recombinant silk composition comprises at least 1 wt % of recombinant silk polypeptide, at about 2 wt %, at about 3 wt %, at about 4 wt %, at about 5 wt %, at about 6 wt %, at about 7 wt %, at about 8 wt %, at about 9 wt %, at about 10 wt %, at about 15 wt %, or at about 20 wt %. In some embodiments, the recombinant silk composition comprises about a 25 wt % solution in glycerin.

In some embodiments, the solvent is water. Without intending to be limited by theory, subjecting the recombinant silk polypeptide to a solvent such as water results in a recombinant silk polypeptide that has expanded or swelled, wherein the protein functions as a carrier containing the solvent such as water. These compositions can be stored dry and partially rehydratable after immersion in water to directly form a liquid or semi-liquid aqueous suspension of expanded particles.

In some embodiments, the recombinant silk protein may expand a portion of the hollow core. In some other embodiments, the recombinant silk protein may expand a portion of the shell. In such embodiments where the solvent is water, the recombinant silk protein transforms into a hydrogel. In other embodiments where the solvent is water, the recombinants silk protein transforms into a paste. In various embodiments, heat and/or pressure may be added to further process the recombinant silk protein compositions.

In some embodiments, a solvent is generally present in a proportion ranging from 55 to 90% by weight relative to the total weight of the recombinant silk polypeptide. This range includes all specific values and subranges therebetween, including 60, 65, 70, 75, 80, and 85% by weight. In some embodiments, the recombinant silk protein is insoluble in various solvents including water at various different pH levels, glycerin, alcohols, siloxane, and oils.

In some embodiments, the solvent is an aqueous type. In such embodiments, the solvent is water. The solvent may have a pH ranging from 6 to 12. In some embodiments, the solvent has a pH of 6. In some other embodiments, the solvent has a pH ranging from 0 to 5, a from 2 to 7, from 4 to 9, from 6 to 11, from 8 to 13, or from 10 to 14.

In other embodiments, the solvent includes a mixture of various volatile organic solvents, in order to obtain relatively short drying times. In some embodiments, the solvent is an alcohol. Solvents may include water, ethyl alcohol, toluene, methylene chloride, isopropanol, n-butyl alcohol, castor oil, organopolysiloxane oils, ethylene glycol monoethyl ether, diethylene glycol monobutyl ether, diethylene glycol monoethyl ether, dimethyl sulphoxide, dimethyl formamide and tetrahydrofuran.

In some embodiments, the organopolysiloxane oil may be volatile, non-volatile, or a mixture of volatile and non-volatile silicones. The term “non-volatile” as used in this context refers to those silicones that are liquid under ambient conditions and have a flash point (under one atmospheric of pressure) of or greater than about 100° C. The term “volatile” as used in this context refers to all other silicone oils. Suitable organopolysiloxanes can be selected from a wide variety of silicones spanning a broad range of volatilities and viscosities. Suitable silicones are disclosed in U.S. Pat. No. 5,069,897, issued Dec. 3, 1991, which is incorporated by reference herein in its entirety. Organopolysiloxanes selected from the group comprising polyalkylsiloxanes, alkyl substituted dimethicones, dimethiconols, polyalkylaryl siloxanes, and mixtures thereof may be used. Polyalkylsiloxanes, dimethicones and cyclomethicones may be used.

In some embodiments, the solvent is a vegetable oil and hydrogenated vegetable oil. In some embodiments, the solvent is a free fatty acid. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, partially and fully hydrogenated oils from the foregoing sources, and mixtures thereof. Animal fats and oils, e.g. cod liver oil, lanolin and derivatives thereof such as acetylated lanolin and isopropyl lanolate may be used. Also useful are C₄-C₂₀ alkyl ethers of polypropylene glycols, C₁-C₂₀ carboxylic acid esters of polypropylene glycols, and di-C₈-C₃₀ alkyl ethers, examples of which include PPG-14 butyl ether, PPG-15 stearyl ether, dioctyl ether, dodecyl octyl ether, and mixtures thereof.

The compositions of the present invention may be substantially free of semi-solid hydrocarbons such as petrolatum, lanolin and lanolin derivatives, sterols (e.g., ethoxylated soya sterols), high molecular weight polybutenes and cocoa butter. By “substantially free,” as used herein, means that the concentration of the semi-solid hydrocarbons is less than 10%, or less than 5% or less than 2% or 0%.

### Recombinant Silk Proteins as a Cosmetics Formulation

In various embodiments, the recombinant silk protein will be compounded into a silk cosmetic or skincare product (e.g., solutions applied to the skin or hair). Specifically, the recombinant silk protein may be used as a base for a cosmetic or skincare product where the recombinant silk polypeptide is present in the base in its monomeric or less-crystalline form. In some embodiments, the recombinant silk protein may be used as a base for a cosmetic or skincare product where the recombinant silk polypeptide is present in the base in a semi-crystalline form. In such embodiments, the recombinant silk polypeptide is not present in the base in its monomeric form.

In most embodiments, the cosmetic formulations are physically stable. In such embodiments, the recombinant silk protein and any other ingredients remain in its formulation for a prolonged period of time, with a prolonged shelf life. On prolonged use, the recombinant silk composition remains substantially stable and the ingredients do not precipitate out of the formulation.

The composition of the invention may be used to apply the silk protein to the skin, nails, hair or mucous membranes, by contacting the composition with the skin, nails, hair or mucous membranes of a subject. Preferably, the inventive composition is used with human subjects.

In most embodiments, the cosmetic formulations are non-toxic, or, non-allergenic to subject hosts to which the cosmetic is applied. It is also desirable in the art to produce cosmetic compositions for hair and epidermal contact which will not permanently stain tissue and which can be removed by ordinary washing with aqueous detergents.

The solids, films, emulsions, hydrogels, and other material forms discussed in various embodiments may contain various humectants, emollients, occlusive agents, active agents, and cosmetic adjuvants, depending on the embodiment and the desire efficacy of the formulation. In some embodiments, the recombinant silk protein functions as a carrier. In some embodiments, the recombinant silk protein is a carrier, delivering one or more agents to a surface such as skin, hair, or nails.

In some embodiments, suitable concentrations of plasticizer by weight in the recombinant silk composition ranges from: 1 to 60% by weight, 10 to 60% by weight, 10 to 50% by weight, 10 to 40% by weight, 15 to 40% by weight, 10 to 30% by weight, or 15 to 30% by weight. In some embodiments, the plasticizer is glycerol. In some embodiments, the plasticizer is triethanolamine, trimethylene glycol, polyethylene glycol, propylene glycol, sorbitol, sucrose, saturated fatty acids, unsaturated fatty acids,

In the instance where water is used as a plasticizer, a suitable concentration of water by weight in the recombinant silk composition ranges from: 5 to 80% by weight, 15 to 70% by weight, 20 to 60% by weight, 25 to 50% by weight, 19 to 43% by weight, or 19 to 27% by weight. Where water is used in combination with another plasticizer, it may be present in the range 5 to 50% by weight, 15 to 43% by weight or 19 to 27% by weight.

In some embodiments, suitable plasticizers may include polyols (e.g., glycerol), water, lactic acid, ascorbic acid, phosphoric acid, ethylene glycol, propylene glycol, triethanolamine, acid acetate, propane-1,3-diol or any combination thereof. In various embodiments, the amount of plasticizer can vary according to the purity and relative composition of the recombinant silk protein. For example, a higher purity powder may have less impurities such as a low molecular weight compounds that may act as plasticizers and therefore require the addition of a higher percentage by weight of plasticizer.

In some embodiments, the recombinant silk compositions comprise humectants or emollients. The term “humectant” as used herein refers to a hygroscopic substance that forms a bond with water molecules. Suitable humectants include, but are not limited to glycerol, propylene glycol, polyethylene glycol, pentalyene glycol, Tremella extract, sorbitol, dicyanamide, sodium lactate, hyaluronic acid, aloe vera extract, alpha-hydroxy acid and pyrrolidinecarboxylate (NaPCA).

The term “emollient” as used herein refers to a compound that provide skin a soft or supple appearance by filling in cracks in the skin surface. Suitable emollients include, but are not limited to shea butter, cocoa butter, squalene, squalane, octyl octanoate, sesame oil, grape seed oil, natural oils containing oleic acid (e.g. sweet almond oil, argan oil, olive oil, avocado oil), natural oils containing gamma linoleic acid (e.g. evening primrose oil, borage oil), natural oils containing linoleic acid (e.g. safflower oil, sunflower oil), or any combination thereof.

The term “occlusive agent” refers to a compound that forms a barrier on the skin surface to retain moisture. In some instances, emollients or humectants may be occlusive agents. Other suitable occlusive agents may include, but are not limited to beeswax, carnuba wax, ceramides, vegetable waxes, lecithin, allantoin. Without intending to be limited by theory, the film-forming capabilities of the recombinant silk compositions presented herein make an occlusive agent that forms a moisture retaining barrier because the recombinant silk polypeptides act attract water molecules and also act as humectants.

The term “active agent” refers to any compound that has a known beneficial effect in skincare formulation or sunscreen. Various active agents may include, but are not limited to acetic acid (i.e. vitamin C), alpha hydroxyl acids, beta hydroxyl acids, zinc oxide, titanium dioxide, retinol, niacinamide, other recombinant proteins (either as full length sequences or hydrolyzed into subsequences or “peptides”), copper peptides, curcuminoids, glycolic acid, hydroquinone, kojic acid, 1-ascorbic acid, alpha lipoic acid, azelaic acid, lactic acid, ferulic acid, mandelic acid, dimethylaminoethanol (DMAE), resveratrol, natural extracts containing antioxidants (e.g. green tea extract, pine tree extract), caffeine, alpha arbutin, coenzyme Q-10, and salicylic acid.

The term “cosmetic adjuvant” refers to various other agents used to create a cosmetic product with commercially desirable properties including without limitation surfactants, emulsifiers, preserving agents and thickeners.

As described below, in various embodiments, the recombinant silk protein may form a semi-solid or gel-like structure that is dispersible. In various embodiments where the recombinant silk protein is compounded into a skin care formulation, the recombinant silk protein may form a non-reversible three-dimensional structure such as a gel or film that transforms into a dispersible liquid upon the surface of the skin.

In various embodiments, the recombinant silk protein may be suspended in water (“aqueous suspended protein”) to form a film, gel, or base that can be incorporated (i.e. compounded) in a cosmetic or skincare formulation. Depending on the embodiment, the amount of recombinant silk protein to water in the aqueous suspended protein can vary, as can the relative ratio of recombinant silk polypeptide powder to additive in the recombinant silk protein. In some embodiments, the protein composition will comprise 10-33% recombinant silk polypeptide powder by weight. In some embodiments, a different solvent than water will be used. In some embodiments, the recombinant silk protein is suspended in water to create an aqueous suspended protein that is 1-40% recombinant silk protein and 60-99% water. In a specific embodiment, the protein composition is suspended in water to create an aqueous suspended protein that is 10% recombinant silk polypeptide powder by weight, 30% additive by weight and 60% water by weight. In a specific embodiment, the protein is suspended in water to create an aqueous suspended protein that is 6% recombinant silk polypeptide powder by weight, 18% additive by weight and 76% water by weight. In a specific embodiment, the protein is suspended in water to create an aqueous suspended protein that is 10% recombinant silk polypeptide powder by weight and 90% water by weight.

Depending on the embodiment, the aqueous suspended protein may be optionally heated and agitated when it is re-suspended in water. In some embodiments, heating and agitating the aqueous suspended protein may result in a phase transformation of the recombinant silk polypeptides in the aqueous suspended protein. Specifically, heating and agitating the aqueous suspended protein results in three distinct phases that are assessed by centrifugation: 1) a gel phase that is distinct from the supernatant after centrifugation; 2) a colloidal phase that can be filtered from the supernatant after centrifugation; and 3) a solution phase that remains after filtering the colloidal phase from the supernatant. Various combinations of heat, agitation and centrifugation may be used, provided that the aqueous suspended protein must not be subject to prolonged heat in order to prevent degradation of the recombinant silk polypeptides. In a specific embodiment, the protein is subjected to gentle agitation at 90° C. for 5 minutes and centrifuged at 16,000 RCF for 30 minutes.

In various embodiments, either the various phases of the aqueous suspended protein (i.e. colloidal phase, gel phase and solution) or the aqueous suspended protein may be incorporated in a cosmetic or skincare formulation to provide a source of recombinant silk protein. Depending on the embodiment, the aqueous suspended protein may subject to agitation with or without heat before incorporating into a skincare formulation. Optionally, the aqueous suspended protein may be separated in the above-discussed phases by centrifugation and/or filtering. Depending on the embodiment, the skincare formulation may be an emulsion (e.g. a cream or serum) or a primarily aqueous solution (e.g. a gel). In certain embodiments, the recombinant silk protein may be incorporated into any of the above-discussed cosmetic, skin care, or hair care formulation without aqueous resuspension. In these compositions, a homogenizer or similar equipment may be used to ensure that the recombinant silk protein is uniformly distributed in the composition.

In some embodiments, the aqueous suspended protein may be subject to heat and agitation, then cast onto a flat surface and dried into a film. In some embodiments, the aqueous suspended protein may be cast onto a flat surface and dried into a film without being subjected to heat and/or agitation. In such embodiments, the aqueous suspended protein may be cast onto a flat surface and dried into a film without being subjected to additional processing. In some embodiments, the aqueous suspended protein may be incorporated into an emulsion, then cast onto a flat surface and dried into a film. Depending on the embodiment, various different drying conditions may be used. Suitable drying conditions include drying at 60° C. or at 80° C. with and without a vacuum. In embodiments that use a vacuum, 15 Hg is a suitable amount of vacuum. Other methods of drying are well established in the art.

In various embodiments, the films comprising the aqueous suspended protein alone have a low melting temperature. In various embodiments, the films comprising the aqueous suspended protein alone have melting temperature that is less than body temperature (around 34-36° C.) and melts upon contact with skin. Without intending to be limited by theory, the recombinant silk polypeptide forms enough intermolecular interactions to make a semi-solid structure (i.e. film), however this structure is reversible upon skin contact and can be re-formed after dispersion on the skin surface. In various embodiments, the film will have reduced crystallinity compared to the recombinant silk protein or the recombinant silk powder, as measured by Fourier-transform infrared spectroscopy (FTIR). In various embodiments, the films comprising the aqueous suspended protein does not melt upon contact with skin. In such embodiments, the film functions as a barrier. In various embodiments, the film is a hydrophobic film of low density. The film or barrier may range from about 1 μm to about 50 μm in thickness, from about 10 μm to about 30 μm, or from about 20 μm to about 40 μm in thickness. Upon contact with skin, the barrier may be formed on the surface of the epidermal layer, materializing a robust, non-specific adherence is made to the skin surface. In some embodiments, the thickness of the film changes depending on the concentration of recombinant silk protein and surface area of application.

In some embodiments, the barrier is long-lasting and prevents against one or more environmental stressors, including wind, humidity, harsh additives, pollution, abrasion, dirt, and grease. The barrier may withstand abrasion equivalent to at least 100 rubs by hand, at least 200 rubs, at least 400 rubs, at least 600 rubs, or at least 800 rubs.

In one specific embodiment, the aqueous suspended protein or the protein may be incorporated (e.g., homogenized) into an emulsion, then cast on a flat surface and lyophilized to create a porous film. Depending on the embodiment, various techniques may be used for lyophilization, including freezing the film at −80° C. for 30 minutes. Other lyophilization techniques will be well known to those skilled in the art.

In various embodiments, the above-described films can be used as a topical skincare agent. This film may be applied directly to the skin and can be re-hydrated to form a dispersible viscous substance that is incorporated into the skin. As discussed herein, various emollients, humectants, active agents and other cosmetic adjuvants may be incorporated into the film. This film may be applied directly to the skin and adsorb to the skin due to contact with the skin, or after gently rubbing the film into the skin. In some embodiments, the film may be applied directly the skin and adsorb to the skin without additional rubbing or contact. In some embodiments, the protein resuspended in an aqueous solution may be applied to the face and then exposed to a coagulant such as propylene glycol via mist to form a gellable mask.

Depending on the embodiment, the film that is cast may be a flat film (i.e. with no surface variability) or may be cast on a mold that incorporates microstructures. In a specific embodiment, the film that is cast on a mold that incorporates microneedle structures to prick the surface of the skin and assist in delivery of active agents.

In an alternate embodiment, the aqueous suspended protein may be added to an emulsion that is used as a cosmetic product. The emulsion may be applied to skin or hair and then allowed to form a film on the surface of the skin upon drying. As discussed below, various emollients, humectants, active agents and other cosmetic adjuvants may be incorporated into the emulsion.

In some embodiments, the recombinant silk compositions may be liquid or semi-solid, such as creams, lotions, and gels. The compositions useful in the subject invention may be made into a wide variety of product forms that are known in the art. These include, but are not limited to, powders, lotions, creams, gels, patches, serums, ampules, powders, sticks, sprays, ointments, pastes, mousses, ointments, liquids, emulsions, foams, or aerosols. These product forms may comprise several types of additives, as further discussed below, including, but not limited to, solutions, aerosols, emulsions, gels, solids, and liposomes. The compounds which are active in the compositions and methods of this invention may be delivered topically by any means known to those of skill in the art.

In some other embodiments, the recombinant silk compositions may be basic cosmetic compositions such as facial cleansers, such as toilet water, cream, essence, cleansing foam and cleansing water, pack and body oil, color cosmetic compositions such as foundation, lipstick, mascara, and make-up base, hair product compositions such as shampoo, rinse, hair conditioner and hair gel, soap, and the like. The cosmetic formulation can be prepared in any method known in the art, using the recombinant silk composition described herein, optionally together with at least one carrier and/or additive, which are commonly used in the field of preparing cosmetic compositions.

In some embodiments, the compositions comprise at least one cosmetic agent. Examples of cosmetic agents include emollients, humectants, colorants, pigments, fragrances, moisturizers, viscosity modifiers and any other cosmetic forming agent. One or more cosmetic agents can be included in the cosmetic composition. In another embodiment, additional active ingredients as known in the art and described herein may also be used, including, but not limited to, a skin softener, a skin permeation enhancer, a colorant, an aromatic, an emulsifier, and a thickener. Also, the cosmetic composition may further comprise a perfumery, a pigment, a bactericidal agent, an antioxidant, a preservative and a moisturizer, and inorganic salts and synthetic polymer substances, for the purpose of improving physical properties.

The composition may also be delivered topically via a lotion. Single emulsion skin care preparations, such as lotions and creams, of the oil-in-water type and water-in-oil type are well-known in the cosmetic art and are useful in the subject invention. Multiphase emulsion compositions, such as the water-in-oil-in-water type are also useful in the subject invention. In general, such single or multiphase emulsions contain water, emollients, and emulsifiers as essential ingredients.

The compositions of the present invention can also be formulated into a solid formulation (e.g., a wax-based stick, soap bar composition, powder, bead, exfoliant, or a wipe containing liquid or powder).

The compositions of this invention can be formulated as a gel (e.g., an aqueous gel using a suitable gelling agent(s)). Suitable gelling agents for aqueous gels include, but are not limited to, natural gums, acrylic acid and acrylate polymers and copolymers, and cellulose derivatives (e.g. hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents. In some embodiments, such compositions include a combination of recombinant silk protein, water (Aqua), sodium C14-16 olefin sulfonate, glycerin, cocoa betaine, sodium benzoate, sodium hydroxide, calcium gluconate, sodium hyaluronate, propanediol, xanthan gum, gluconolactone, and tetrasodium glutamate diacetate. In some embodiments, compositions comprise a cleansing detergent, soap, serum, or toner. In a specific embodiment, the serum is aqueous-based. In another specific embodiment, the toner is alcohol-based.

The compositions useful in the present invention may be formulated as emulsions. If the composition is an emulsion, from about 1% to about 10% or from about 2% to about 5% of the composition comprises an emulsifier. Emulsifiers may be nonionic, anionic or cationic. Suitable emulsifiers are disclosed in, for example, INCI Handbook, pp. 1673-1686. Lotions and creams can be formulated as emulsions.

Yet another type of composition may be an ointment. An ointment may comprise a simple base of animal or vegetable oils or semi-solid hydrocarbons. An ointment may comprise from about 2% to about 10% of an emollient in addition to from about 0.1% to about 2% of a thickening agent. Examples of thickening agents include cellulose derivatives (methyl cellulose and hydroxyl propylmethylcellulose), synthetic high molecular weight polymers (e.g., carboxyvinyl polymer and polyvinyl alcohol), plant hydrocolloids (e.g., karaya gum and tragacanth gum), clay thickeners (e.g., colloidal magnesium aluminum silicate and bentonite), and carboxyvinyl polymers, carboxylic acid polymers, crosslinked polyacrylates, polyacrylamides, xanthan gum and mixtures thereof.

The compositions useful in the subject invention may contain, in addition to the aforementioned components, a wide variety of additional oil-soluble materials and/or water-soluble materials conventionally used in compositions for use on skin, hair, and nails at their art-established levels.

The compositions of the present invention may be directed applied to the skin or may be applied onto other delivery implements such as wipes, sponges, brushes, and the like. The compositions may be used in products designed to be left on the skin, wiped from the skin, or rinsed off of the skin.

In some embodiments, the composition improves the appearance of skin, such as increasing skin firmness/plumpness, increasing elasticity, improving overall skin health, increasing hydration, accelerating and/or improving wound healing, improving pollution defense, reducing dermatological aging, decreasing skin fragility, preventing and reversing loss of collagen and/or elastin, preventing skin atrophy, promoting/accelerating cell turnover, increasing genetic expression, improving skin texture, preventing and decreasing fine lines and wrinkles, improving skin tone, enhancing skin thickness, decreasing pore size, minimizing skin discoloration, restoring skin luster, minimizing signs of fatigue, improving skin barrier function, minimizing skin dryness, preventing, reducing, or treating hyperpigmentation, improving the mitochondrial function of the skin, improves exfoliation, reduces toxicity, mattifying skin, reducing oxidative stress levels, attenuating pollution induced oxidative stress, attenuating UVA or UVB induced oxidative stress, and any combination thereof.

The compositions of various embodiments defend against pollutants and other irritants. As a result, many skin conditions, such as acne, the redness associated with rosacea (adult acne), and other inflammatory conditions can be actively managed by application of the cosmetic formulations.

### Coagulants

In some embodiments, a silk-based composition produced herein is exposed to a coagulant. This can change the properties of the composition to facilitate controlled aggregation of silk in the silk-based composition. In some embodiments, the silk-based composition is submerged in a coagulant. In some embodiments, the silk-based composition is exposed to a coagulant mist or vapor. In one embodiment, an aqueous protein composition comprises or is submerged with or mixed with a coagulant. In some embodiments, a silk-based solid or semi-solid, such as a film is submerged in or exposed to a vapor comprising coagulant. In some embodiments, methanol is used as an effective coagulant.

In some embodiments, alcohol can be used as a coagulant or solvent, such as isopropanol, ethanol, or methanol. In some embodiments, 60%, 70%, 80%, 90% or 100% alcohol is used as a coagulant. In some embodiments, a salt can be used as a coagulant, such as ammonium sulfate, sodium chloride, sodium sulfate, or other protein precipitating salts effective at a temperature from 20 to 60° C.

In some embodiments, a combination of one or more of water, acids, solvents and salts, including, but not limited to the following classes of chemicals of Brönsted-Lowry acids, Lewis acids, binary hydride acids, organic acids, metal cation acids, organic solvents, inorganic solvents, alkali metal salts, and alkaline earth metal salts can be used as a coagulant. In some embodiments, the acids comprise dilute hydrochloric acid, dilute sulfuric acid, formic acid or acetic acid. In some embodiments the solvents comprise ethanol, methanol, isopropanol, t-butyl alcohol, ethyl acetate, propylene glycol, or ethylene glycol. In some embodiments, the salts comprise LiCl, KCl, BeCl₂, MgCl₂, CaCl₂, NaCl, ZnCl₂, FeCl₃, ammonium sulfate, sodium sulfate, sodium acetate, and other salts of nitrates, sulfates or phosphates. In some embodiments, the coagulant is at a pH from 2.5 to 7.5.

### Other Additives

In some embodiments, a silk-based composition produced herein is exposed to other additives. This can change the properties of the composition as it interacts with the skin. In some embodiments, the silk-based composition is submerged in the additive. In some embodiments, the silk-based composition is exposed to the additive mist or vapor. In one embodiment, an aqueous protein composition comprises or is submerged with or mixed with the additive. In some embodiments, a silk-based solid or semi-solid, such as a film is submerged in or exposed to a vapor comprising the additive. In some embodiments the silk-based gel is exposed to the additive prior to hallow powder formation (e.g. the silk-based gel and additive are co-spray dried together).

The additive can itself be inert or it can possess dermatological benefits of its own. The additive should also be physically and, chemically compatible with the essential components described herein, and should not unduly impair stability, efficacy or other use benefits associated with the compositions of the present invention. The type of additive utilized in the present invention depends on the type of product form desired for the composition. In some embodiments, the additive is an acid textile dye.

Pigments are frequently added to cosmetic formulations to achieve a desired color for application to the skin. Such pigments are known and the concentrations required to achieve a desired coloring are readily determinable. Pigments may be inorganic or organic. Inorganic pigments include iron oxides (red, black, brown colors), manganese violet, ultramarines (green, blue, pink, red, or violet aluminum sulfosilicates), aquamarines, copper powder, mica, clays, silica, and titanium dioxide. Organic dyes that have been certified by the US FDA for cosmetic use generally have the prefix “D&C” and a suffix of a color and a number (for example, D&C Green #3).

Certain embodiments of the present invention contain from about 0% to about 30%, about 1% to about 20%, from about 2% to about 15% or from about 5% to about 15% of a colorant, on an anhydrous pigment weight basis. These are usually aluminum, barium or calcium salts or lakes. Dyes may be present at a concentration of from about 0% to about 3% and pearlizing agents and the like from 0% to about 10%. Such dyes in combination with recombinant silk proteins are stable and have a long shelf-life. The shelf-life of such compositions may be about 6 months, about 1 year, or about 2 years. In some embodiments, the shelf-life of such compositions may be at least 5 years.

There are no specific limitations as to the pigment, colorant, or filler powders used in the composition. Each may be a body pigment, inorganic white pigment, inorganic colored pigment, pearling agent, and the like. Specific examples are talc, mica, magnesium carbonate, calcium carbonate, magnesium silicate, aluminum magnesium silicate, silica, titanium dioxide, zinc oxide, red iron oxide, yellow iron oxide, black iron oxide, ultramarine, polyethylene powder, methacrylate powder, polystyrene powder, silk powder, crystalline cellulose, starch, titanated mica, iron oxide titanated mica, bismuth oxychloride, and the like.

Additional pigment/powder fillers include, but are not limited to, inorganic powders such as gums, chalk, Fuller's earth, kaolin, sericite, muscovite, phlogopite, synthetic mica, lepidolite, biotite, lithia mica, vermiculite, aluminum silicate, starch, smectite clays, alkyl and/or trialkyl aryl ammonium smectites, chemically modified magnesium aluminum silicate, organically modified montmorillonite clay, hydrated aluminum silicate, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicone dioxide, and boron nitride; organic powder such as polyamide resin powder (nylon powder), cyclodextrin, methyl polymethacrylate powder, copolymer powder of styrene and acrylic acid, benzoguanamine resin powder, poly(ethylene tetrafluoride) powder, and carboxyvinyl polymer, cellulose powder such as hydroxyethyl cellulose and sodium carboxymethyl cellulose, ethylene glycol monostearate; inorganic white pigments such as magnesium oxide. Other useful powders are disclosed in U.S. Pat. No. 5,688,831, to El-Nokaly et al., issued Nov. 18, 1997, herein incorporated by reference in its entirety. These pigments and powders can be used independently or in combination.

Besides the silk protein, the composition according to the invention can further comprise a film-forming substance. Examples of film-forming substances include cellulose derivatives, nitrocellulose, acrylic polymers or copolymers, acrylic, styrene, acrylate-styrene and vinyl resins, vinyl copolymers, polyester polymers, arylsulphonamide resins and alkyde resins.

In some embodiments, the composition may include an amphoteric surfactant, a phospholipid, or a wax.

Examples of other additives include, but are not limited to, cannabidiol, foaming surfactants, depigmentation agents, reflectants, detangling/wet combing agents, amino acids and their derivatives, antimicrobial agents, allergy inhibitors, anti-acne agents, anti-aging agents, anti-wrinkling agents antiseptics, analgesics, antitussives, antipruritics, local anesthetics, anti-hair loss agents, hair growth promoting agents, hair growth inhibitor agents, antihistamines, antiinfectives, inflammation inhibitors, anti-emetics, anticholinergics, vasoconstrictors, vasodilators, wound healing promoters, peptides, polypeptides and proteins, deodorants and antiperspirants, medicament agents, skin emollients and skin moisturizers, skin firming agents, hair conditioners, hair softeners, hair moisturizers, vitamins, tanning agents, skin lightening agents, antifungals, depilating agents, shaving preparations, external analgesics, perfumes, counterirritants, hemorrhoidals, insecticides, poison ivy products, poison oak products, burn products, anti-diaper rash agents, prickly heat agents, make-up preparations, vitamins, herbal extracts, retinoids, flavenoids, sensates, anti-oxidants, skin conditioners, hair lighteners, chelating agents, cell turnover enhancers, sunscreens, anti-edema agents, collagen enhancers, and mixtures thereof.

Examples of suitable vitamins nonexclusively include vitamin B complex, including thiamine, nicotinic acid, biotin, pantothenic acid, choline, riboflavin, vitamin B6, vitamin B12, pyridoxine, inositol, camitine; vitamins A, C, D, E, K and their derivatives such as vitamin A palmitate and pro-vitamins, e.g. (i.e. panthenol (pro vitamin B5) and panthenol triacetate) and mixtures thereof.

Examples of sunscreen agents include, but are not limited to, avobenzone, benzophenones, bornelone, butyl paba, cinnamidopropyl trimethyl ammonium chloride, disodium distyrylbiphenyl disulfonate, paba, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, Padimate O, red petrolatum, and mixtures thereof.

The amount of additive to be combined with the recombinant silk composition may vary depending upon, for example, the ability of the additive to penetrate through the skin, hair or nail, the specific additive chosen, the particular benefit desired, the sensitivity of the user to the additive, the health condition, age, and skin, hair, and/or nail condition of the user, and the like. In sum, the additive is used in a “safe and effective amount,” which is an amount that is high enough to deliver a desired skin, hair, or nail benefit or to modify a certain condition to be treated, but is low enough to avoid serious side effects, at a reasonable risk to benefit ratio within the scope of sound medical judgment.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLES

### Example 1: Recombinant 18B Polypeptide Powder Morphology

Recombinant 18B polypeptide (SEQ ID NO: 1) having the FLAG tag was produced through various lots of large-scale fermentation, recovered, and dried into a powder. Details of preparation of the samples are provided below.

Specifically, genetically modified yeast cells produced recombinant 18B polypeptide, as disclosed in International Publication No. WO/2015/042164, “Methods and Compositions for Synthesizing Improved Silk Fibers,” incorporated by reference in its entirety, and were cultured in aerobic fermenters. In this example, the fermenters used ranged from 3,000 L to 26,000 L in volume. The fermentation was run for 72 hours and the process contained a temperature shift in which the batch phase was held at 30° C. for the first 5-8 hours before it was dropped to 25° C. when the glucose feed was triggered. The fermentation process began with 15 g/L of batch glucose, which was consumed within the first 5-8 hours. After the glucose was depleted, an exponential feed was triggered until the oxygen uptake rate (OUR) reached 115 mmol O₂/L/h. The feed rate was then adjusted accordingly to maintain the OUR at 115-120 mmol O₂/L/h. Ethanol accumulation was limited by maintaining the respiratory quotient at about 1.02. In addition, dissolved oxygen was also controlled at 5% of saturation with an agitation cascade. After 72 hours of fermentation, a whole cell broth (WCB) was produced that contained the 18B polypeptide along with spent media which included dead yeast and its metabolites. 8 M urea was combined with the WCB to solubilize the 18B polypeptide into the aqueous phase. The aqueous phase was then separated from the cells via centrifugation. The centrate, which contained the dissolved silk protein, was concentrated and washed by ultrafiltration to remove urea and some impurities. The protein was further purified with two precipitation steps using 10 wt % sodium sulfate solution. In each case, the target protein was precipitated then recovered by centrifugation in the heavy phase. The salt was washed out and the protein was concentrated by ultrafiltration or microfiltration resulting in a protein solids slurry in water. The slurry was then spray dried to the final 18B powder form.

Spray drying was performed as follows: Chilled retentate was fed to a tall form spray dryer via a two-fluid or pneumatic nozzle. The atomized protein slurry was dried co-currently with hot dry air and the majority of the protein powder was collected in the cyclone. The outlet temperature at the cyclone was controlled at 99° C.±2° C. and a relative humidity of <12%. These conditions ensured the powder collected in the cyclone was maintained at or below 3 wt % moisture. Powder that was not captured at the cyclone was collected in a baghouse.

The operating parameters at the dryer were controlled to optimize yield and produce a median particle size greater than 20 m but less than 50 m. These parameters include, but are not limited to, the air to liquid feed rate ratio (ALR) at the atomizing nozzle, the hot air inlet temperature and flow rate, and the solids concentration of the retentate. Deviation from target parameters could result in insufficient drying, significant powder loss to deposition on dryer wall, or significant powder loss to the baghouse.

After the cyclone, powder was passed through a 60-mesh sieve to remove larger particles then collected and bagged as final product. Care was taken to minimize exposure to moist, ambient air while the powder is cooling.

Powder collected from the baghouse is often of insufficient quality compared to the cyclone in that moisture content is generally greater than 3% and the 18B protein content is lower. Therefore, baghouse powder was collected separately from the cyclone product.

As shown in FIG. 1A, the 18B powder in the dry and hydrated state exists as a hollow particle. This morphology was evidenced by both scanning electron microscopy (SEM) and light/polarized microscopy. SEM used a focus electron beam to assess the morphology of materials through the secondary electrons. The electron beam was scanned in a raster pattern to collect micrographs at scales between 1 mm and 10 nm or between 10× and 100,000× magnification. The SEM method used low vacuum (1 to 10 torr), avoiding the need for dehydrating or sputter coating biological samples.

Polarized Light Microscopy was also used to examine the powder morphology. Light and polarized light images were obtained using a Leica DM750P polarized light microscope with a 4× objective. The microscope was coupled to the complementary PC based image analysis Leica Application Suite, LAS V4.9. For polarized microscopy, the Analyzer/Bertrand Lens module was engaged by flipping the lower rocker of the module to the right (the “A” position/Analyzer in), while ensuring the upper rocker of the Analyzer/Bertrand Lens Module was flipped to the left (the “O” position/Bertrand Lens out). This set up allowed for analysis in “cross-polarization mode” which is a state of optical alignment where the allowed oscillatory directions of the light passing through the polarizer and analyzer are oriented at 90°.

FIG. 1A shows SEM images of intact and cracked powder particles in the dry state. When the powder particle was cracked open, it revealed a visible hollow core and thin membrane outer shell. The diameter of the intact powder particle was approximately 100 μm in diameter.

FIG. 1B shows light and polarized microscopy images of the hollow shell morphology of 18B powder in the hydrated state. Maltese crosses were observed on only the outer edges of a powder particle. A solid particle would display a maltese cross through the entire thickness of the powder particle.

To assess the degradation of 18B powder, 18B powder was subjected to various solvents. 18B powder was not soluble in water, as determined by visual inspection and size exclusion chromatography (HPLC-SEC) and reverse phase (RP-HPLC). In this example, 18B powder was dispersed in solvents at 1-10 wt %. The solutions were incubated for 24 hours at 22° C. The powder was pelleted out with centrifugation at 16,000 RCF for 15 minutes at 4° C. The supernatant was poured off the pellet. Both supernatant and pellet were measured for 18B powder content with HPLC-SEC and Reverse Phase. For HPLC-SEC, samples were dissolved in 5 M Guanidine Thiocyanate and injected onto a Yarra SEC-3000 SEC-HPLC column to separate constituents on the basis of molecular weight. Refractive index was used as the detection modality. 18B aggregates, 18B monomer, low molecular weight (1-8 kDa) impurities, intermediate molecular weight impurities (8-50 kDa) and high molecular weight impurities (110-150 kDa) were quantified. Relevant composition was reported as mass % and area %. BSA was used as a general protein standard with the assumption that >90% of all proteins demonstrated dn/dc values (the response factor of refractive index) within about 7% of each other. Poly(ethylene oxide) was used as a retention time standard, and a BSA calibrator was used as a check standard to ensure consistent performance of the method. As a result, the 18B powder was insoluble in water.

Reverse Phase High Performance Liquid Chromatography (“RP-HPLC”) was used to measure the amount by weight of 18B polypeptide monomer in the powder. The samples were dissolved using a 5 M Guanidine Thiocyanate (GdSCN) reagent and injected onto an Agilent Poroshell 300SB C3 2.1×75 mm 5 μm column to separate constituents on the basis of hydrophobicity. The detection modality was UV absorbance of peptide bond at 215 nm (360 nm reference). The sample concentration of 18B-FLAG monomer was determined by comparison with an 18B-FLAG powder standard, for which the 18B-FLAG monomer concentration had been previously determined using Size Exclusion Chromatography (SEC-HPLC). A high level of non-degraded 18B monomer was observed.

### Example 2: Expansion of Recombinant 18B Polypeptide Powder in Various Solvents

The 18B powder, as produced according to the methods described in Example 1, yielded different expanding or swelling depending on the solvent used to disperse the powder. 18B powder may be dispersed in various solvents including aqueous solvents, oils, or silicones.

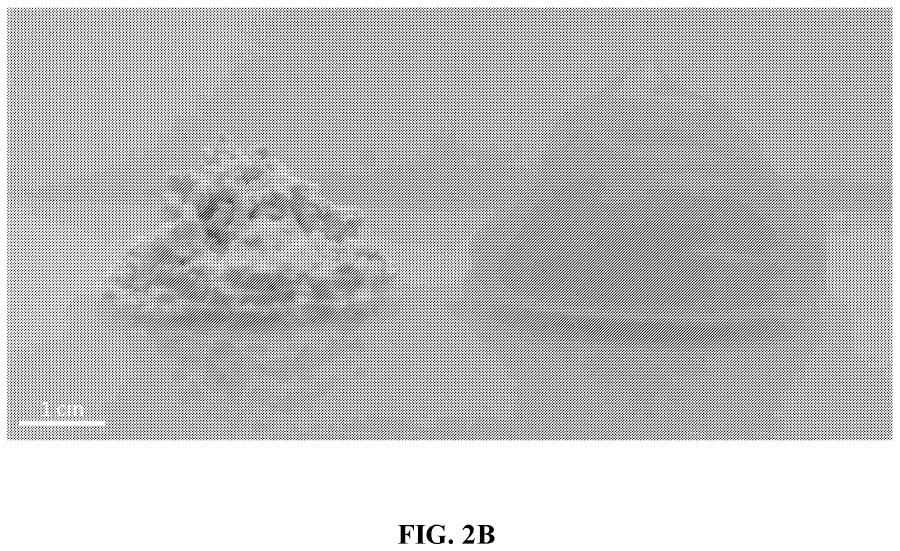

The powder particles exhibited differences in swelling depending on the solvent used to disperse the powder. FIG. 2A shows light microscopy images of 18B powder resuspended in various different solvents including water with a pH of 6 and glycerin, as compared to 18B powder without a solvent. Light microscopy images were taken by suspending 18B powder in different solvents at 1 wt % levels. To make the solution, powder was massed on a scale and then poured into a mixing vessel, such as a 50 mL conical tube. Then, the solvent was dispensed over the powder using a pipette. The mixing vessel was then rigorously shaken by hand, by vortex, or by planetary mixer. A droplet of the 18B powder suspensions was loaded onto a glass slide and covered with a glass coverslip. Light microscopy images were obtained using a Leica DM750P light microscope with a 10× objective. The microscope was coupled to the complementary PC based image analysis Leica Application Suite, LAS V4.9.

FIG. 2B shows a photograph of 18B powder in a dry state and 18B powder after exposure to an aqueous solution. The 18B powder noticeably expanded, taking on about 6 to 10 times its own weight in water. Quantification of percent powder diameter also increased in water. Quantification of the powder diameter was conducted using ImageJ software and the particle analyzer function. Dry powder ranged from 5 to 25 μm in diameter, while hydrated powder in water ranged from 20 to 80 μm in diameter.

### Example 3: Generation of Dyed Recombinant 18B Polypeptide Powder

To assess the dyeing of 18B powder, as produced according to the methods described in Example 1, acid textile dye was prepared according to manufacturer directions, mixed with 18B powder, and incubated for more than 5 minutes. To rinse away excess dye, the sample was centrifuged at 16,000 RCF for 15 minutes at 4° C. The supernatant was poured off and the dyed powder pelleted at the bottom of the tube. Deionized (DI) water was added to the pellet and the pellet was resuspended. This process was repeated several times until the supernatant was clear.

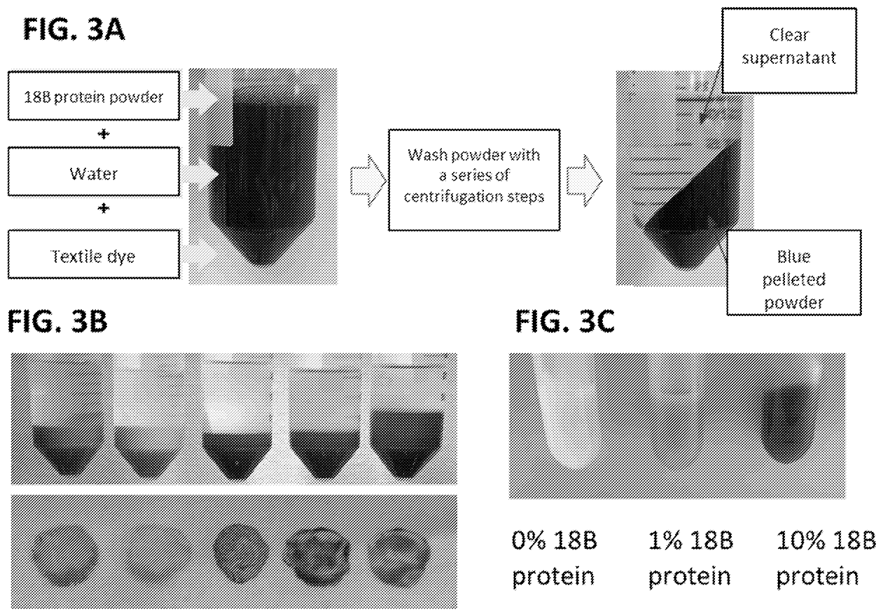

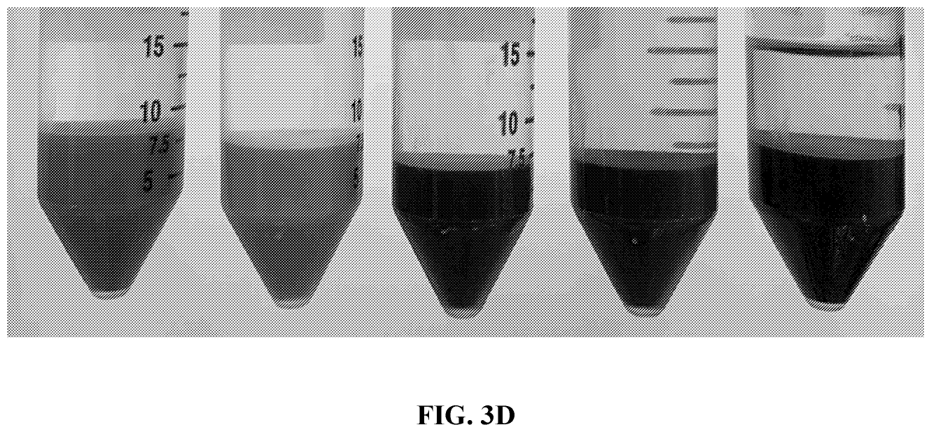

FIG. 3A shows a schematic diagram of the generation of dyed 18B powder using a mixture of water, acid textile dye, and 18B powder. Such a mixture resulted in dyed 18B powder, which easily and rapidly absorbed the vibrant color.

FIG. 3B shows that 18B powder can be dyed at the final powder state. 18B powder was also dyed prior to spray drying or the final powder state. A slurry of colored powder applied directly to the skin showed the vibrancy of the color.

FIG. 3C shows different concentrations of dyed 18B powder (0 wt %, 1 wt %, and 2 wt %) added to cream emulsions. Evidently, the dyed 18B powder could add color to cream emulsions and the vibrancy was determined by 18B powder concentration.

FIG. 3D shows the stability of color fastness of dyed 18B powder after 6 months of storage at 4° C. No leaching was seen after 6 months of storage at 4° C.

### Example 4: Formation of a Film from Recombinant 18B Polypeptide Powder

To assess formation of a film from 18B powder, as produced according to the methods described in Example 1, silk solutions were prepared by dispersing 18B powder in DI water at 1 wt % with gentle shaking. Films were cast on polydimethylsiloxane substrates and left at ambient conditions (22° C. and 40% humidity) over night. Films were cracked and then imaged via SEM. The acid textile dye was prepared according to the methods described in Example 3.

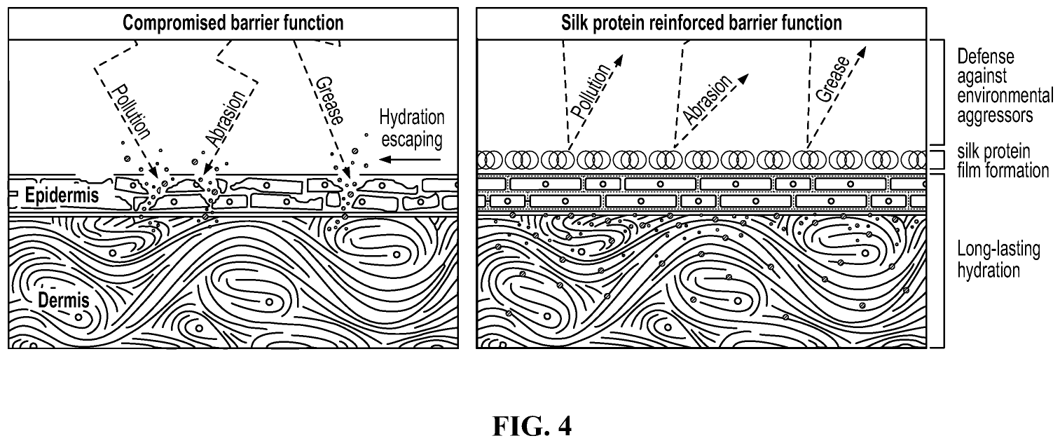

The schematic diagram in FIG. 4 suggests that 18B powder as a hydrated solution, such as a 1 wt % 18B powder solution, dries down on the skin to form a thin homogeneous barrier on the surface of the epidermal layer, which is highly substantive (i.e. a robust, non-specific adherence is made to the skin surface) to skin, and in doing so can provide defense to the skin (the “18B powder barrier”). The 18B powder acts as a barrier to reinforce and strengthen the vital barrier function of the outermost dermal layer. This mechanistic model is compared to skin without such a barrier, where the skin is compromised by environmental stressors including pollution, abrasion, dirt, and grease.

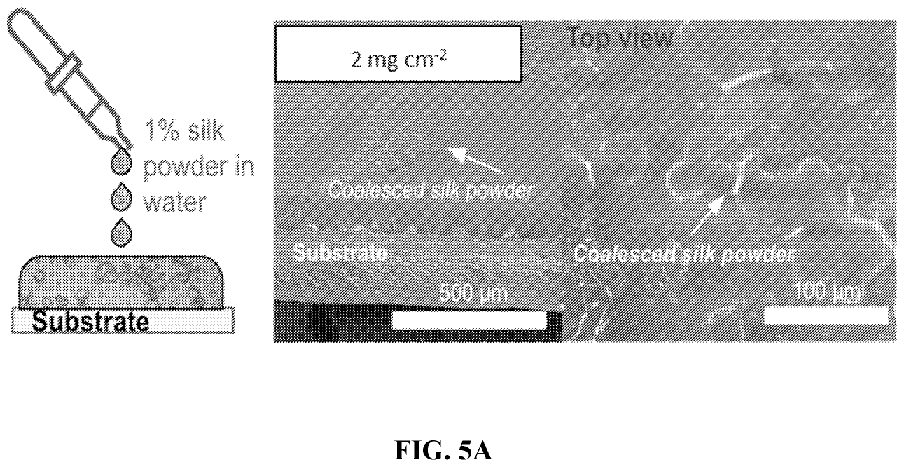

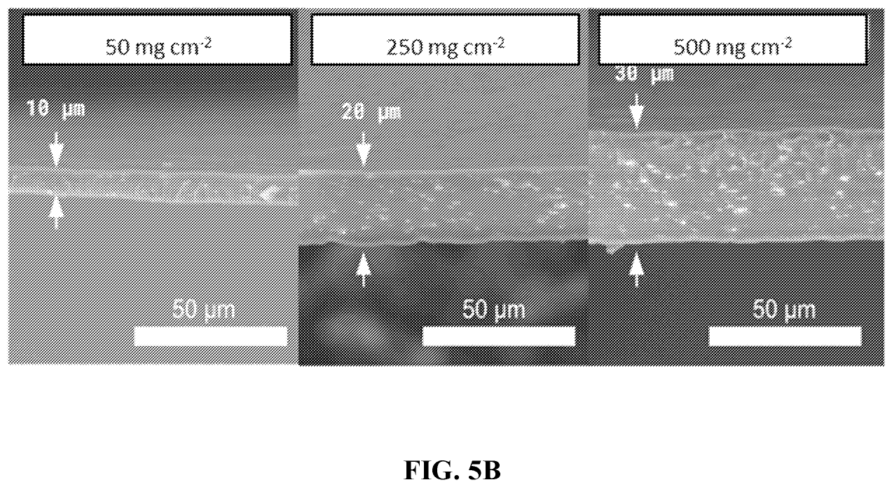

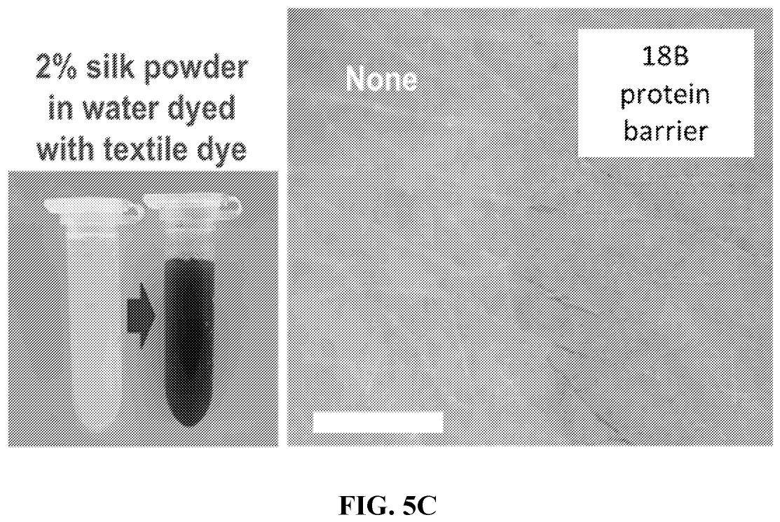

FIG. 5A shows a schematic diagram of the methods described in this example, and SEM images of dried 1 wt % 18B powder solution coalescing into a thin film of about 1 μm thickness when applied to the skin at 2 mg/cm². As the hydrated 18B powder dried, the particles noticeably coalesced into a thin film, as indicated by arrows in FIG. 5A.

FIG. 5B shows the thickness of film changing depending on the solution concentration and surface area. Dried 1 wt % 18B powder solution applied to the skin at 50 mg/cm² yielded a film of about 10 μm thickness. Dried 1 wt % 18B powder solution applied to the skin at 250 mg/cm² yielded a film of about 20 μm thickness. Dried 1 wt % 18B powder solution applied to the skin at 500 mg/cm² yielded a film of about 30 μm thickness.

FIG. 5C shows a 2 wt % 18B powder solution dyed with textile dye and images of the skin before and after dyed 2 wt % 18B powder has been applied at 2 mg/cm² and washed to remove unbound color. Specifically, when a 2 wt % 18B powder solution was applied to skin at 2 mg/cm², a homogeneous coating was observed.

### Example 5: Recombinant 18B Polypeptide Powder as a Long-Lasting Barrier

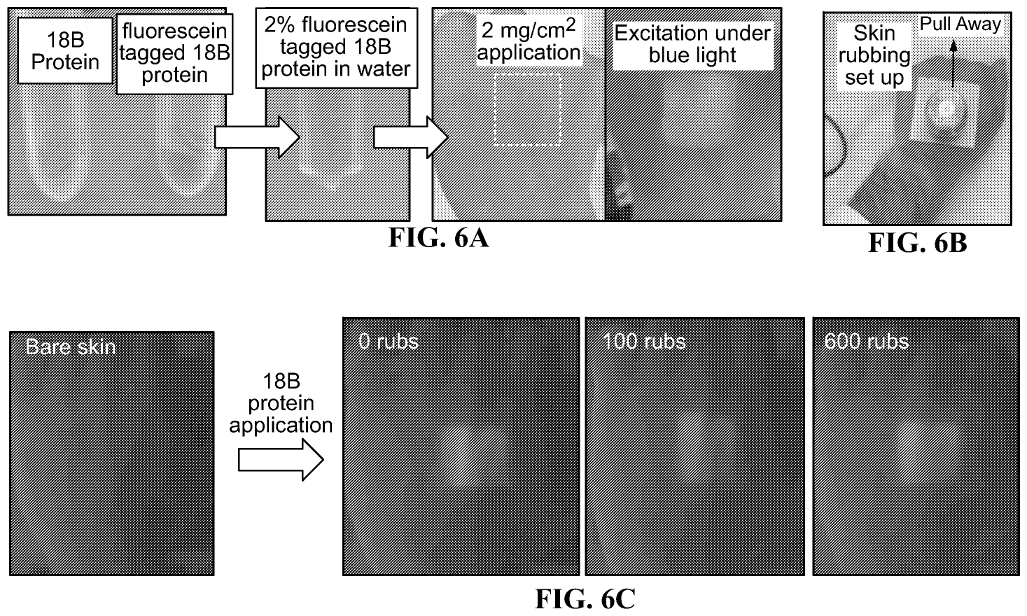

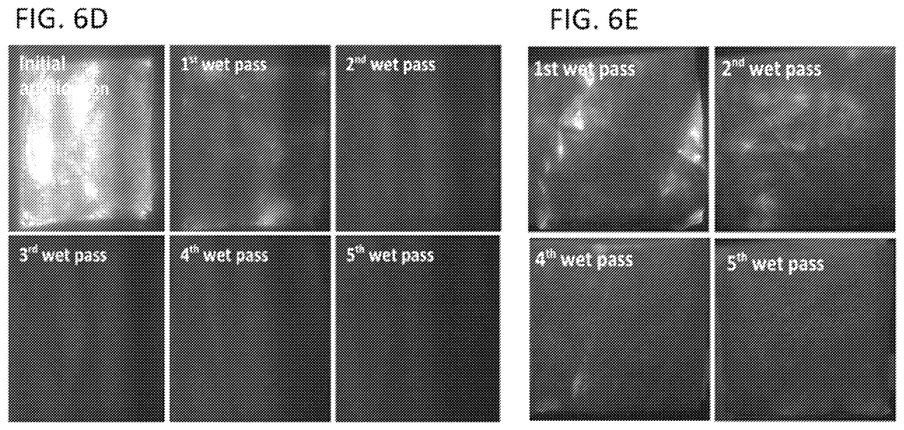

To assess the long-term benefits of 18B powder, as produced according to the methods described in Example 1, the 18B powder barrier was visualized by fluorescently tagging the protein as shown in FIG. 6A.

FIG. 6B shows an experimental design to investigate the effect of repeated abrasion on an 18B powder barrier. A fluorescently tagged 18B powder barrier was applied to the skin and dried for no more than 10 minutes. A 200 g mass was placed over a white, cotton wipe and dragged over the back of the hand for a designated number of times (or “rubs”).

FIG. 6C shows images of an 18B powder barrier subjected to repeated abrasion of no rubs, 100 rubs, and 600 rubs, as compared to bare skin (“control”). The 18B powder barrier was highly substantial to skin, even after exposure to repeated abrasion. As can be seen from the images, the 18B powder barrier can be visualized after 100 rubs and even after 600 rubs, almost substantially intact.

FIG. 6D shows images of an 18B powder barrier on the skin after one to five passes of a wet wipe. As shown, the 18B powder barrier was easily removed with water after minimal washing.

FIG. 6E shows images of the wet wipe after minimal and gentle multiple passes, including a first, second, fourth, and fifth pass. As shown, the wet wipe completely removed the silk-based barrier within only a few passes. The findings suggested that the silk-based film withstood repeated abrasion insults that mimic everyday wear and tear on the skin. No aggressive rubbing or harsh solvent were needed to remove the film. This meant the film did not build up over time or create an unpleasant aesthetic. The film also did not disrupt the skin's natural barrier function (e.g., clog pores). These benefits highlight that the 18B protein barrier was robust to abrasion but was also easily wiped off, which indicated that it did not build up over time, which is a common problem with some polymers.

To produce the fluorescent silk powder, 18B powder was combined with borate buffer (25 mg of 18B powder into 5 mL of buffer). NHS-fluorescein (3 mg, Thermo Fisher) was dissolved in 300 uL of DMSO. 216 uL of the DSMO-dye solution was combined with the silk solution. This mixture was incubated for 1 hour and then dialyzed against water for 24 hours. The dialyzed solution was collected and centrifuged at 16,000 RCF for 20 minutes at 4° C. The supernatant was poured off and more water was replaced. The pellet was dislodged through rigorous shaking to resuspend the pellet. The centrifugation and supernatant exchange steps were repeated a total of four more times. The resulting silk pellet was lyophilized and grounded into a powder. 18B powder was then resuspended to make a 2 wt % silk solution in DI water and applied to an area on the back of the hand at 2 mg/cm² and allowed to dry down for 5-10 minutes. The area on the back of the hand was imaged by exciting the 18B protein barrier with a blue light (467-498 nm) and viewing the reflected light with a 513-556 nm filter. The captured image was converted to black and white to assess intensity of the 18B protein barrier. To evaluate the washability of the 18B protein barrier from skin, a wet wipe (Water Wipes) was passed over area on the back of the hand, multiple times. A new wet wipe was used with each pass. The back of the hand and each wipe was imaged after each pass.

### Example 6: Use of Recombinant 18B Polypeptide Powder for Improving Pollution Wash-Off

To assess the defensive benefits of 18B powder, as produced according to the methods described in Example 1, a pollution rating study was performed. It was determined that 18B powder showed benefits for improving pollution wash off.

In the study, a total of 5 female subjects between the ages of 35 and 56 years with Fitzpatrick skin types I, II, III or IV were enrolled into the study. All five subjects successfully completed the test procedure. At baseline or post-application and pre-rinse, a trained technician marked 1.5 in² test sites on the right or left forearm of each subject and applied approximately 0.3 grams of the test article or 2 wt % 18B powder solution in water to one designated test site and allowed it to air dry for 10 minutes. The test articles were prepared with DI water and 1% preservative (Biotinistat). Application of environmental pollutant or carbon was then sprayed onto the test site containing the test article and also onto the remaining untreated control test site. The technician applied a sufficient amount of carbon or dirt to cover each test site so that each site had a visible dirt score of at least a marked level 3, as indicated below. The score had to be equal on both test sites. The technician and subject evaluations of visible dirt were then recorded. Following the post-application-pre-rinse evaluations, the technician then rinsed each test site with tepid water for 45 seconds and evaluations were repeated. A decrease in evaluation scores indicated an improvement or a decrease in visible dirt. An increase indicated a worsening.

The rating levels used in this trial were as follows: 0=no dirt; 1=slight dirt present; 2=Moderate dirt present (moderately visible); 3=Marked dirt present (very visible); and 4=Severe dirt present (extremely visible).

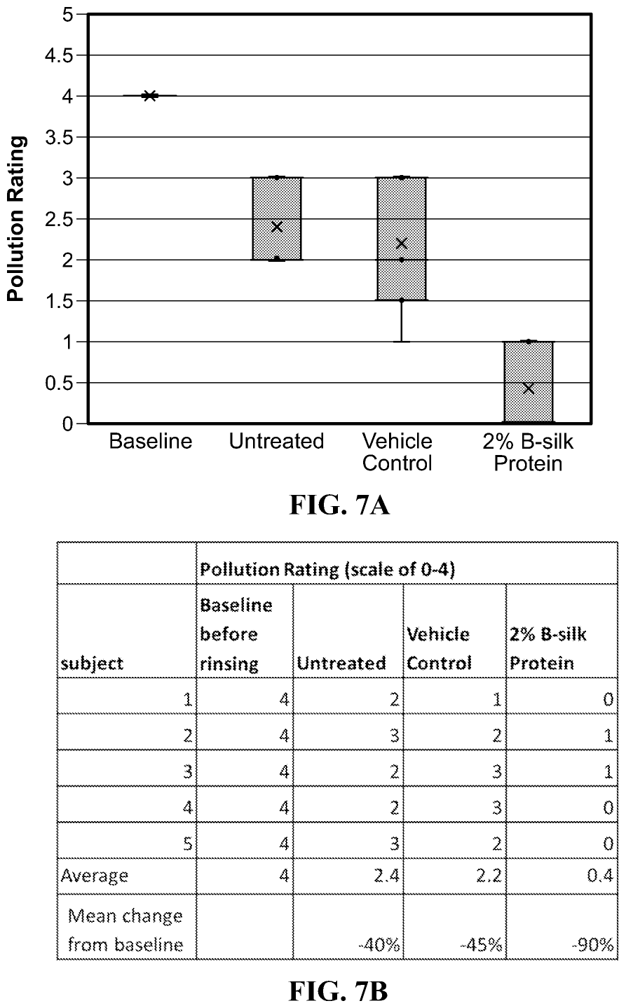

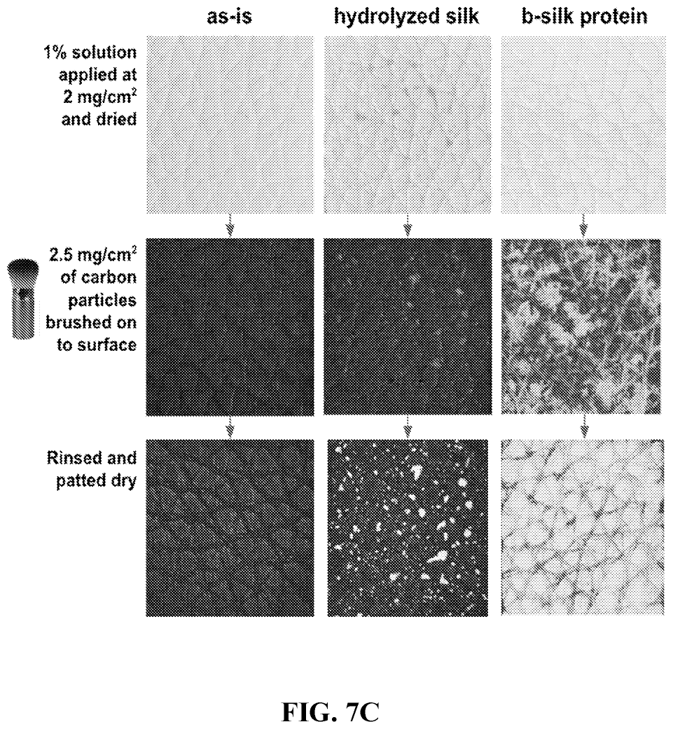

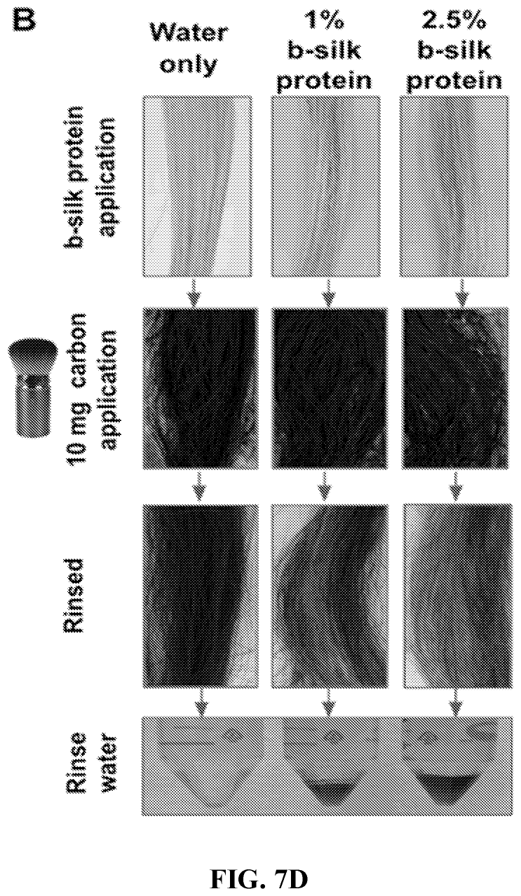

FIGS. 7A and 7B show the results of the pollution rating study to investigate the effects of an 18B powder solution on carbon particles. In this third-party, double blind, vehicle controlled clinical study, where 18B powder was suspended in DI water at 2 wt % 18B powder, the 18B powder solution exhibited a 90% improvement against visible carbon particles observed by the technician compared to baseline, with an average rating level of 0.4. These findings were in contrast to the untreated site and the vehicle control site, which exhibited a 40% and 45% improvement, respectively against visible carbon particles observed by the technician compared to baseline. The average rating level for the untreated site was 2.4 and for the vehicle control site was 2.2. Based on the results, 18B powder solution formed a breathable barrier on the skin and acted as a vital defense against environmental stressors.

FIG. 7C shows images of pollution washes performed on polyurethane material or faux skin using hydrolyzed silk and an 18B powder solution, as compared to a control. Specifically, 1 wt % 18B powder solution was applied on the faux skin at 2 mg/cm² and then dried. After, 2.5 mg/cm² of carbon particles were brushed on the surface. Lastly, the faux skin was rinsed and patted dry. Unlike hydrolyzed silk, 18B powder exhibited the ability to resist pollution adsorption. 18B powder coated onto a skin substitute, showed more long-range film formation, minimal pollution adsorption, and improved pollution wash-off properties.

FIG. 7D shows images of pollution washes performed on hair using 1 wt % and 2.5 wt % 18B powder solution, as compared to a control, and the resultant rinse water after the washings. 18B powder improved the removal of pollution from skin and hair when rinsed. Two solutions of 18B powder at 1 wt % and 2.5 wt % were applied to hair. Then, 10 mg of carbon was added. After rinsing, the rinse water was centrifuged at 16,000 RCF for 15 minutes and observed. It was evident from inspecting the hair coloration and the carbon particle pellet size in the rinse water that hair with 18B powder showed increased pollution removal with increasing 18B powder content.

### Example 7: Recombinant 18B Powder Formulated into a Cleanser

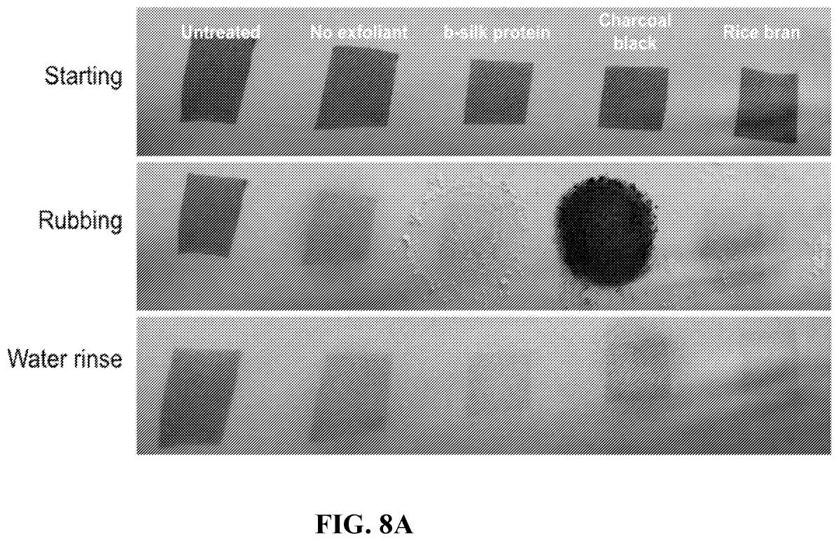

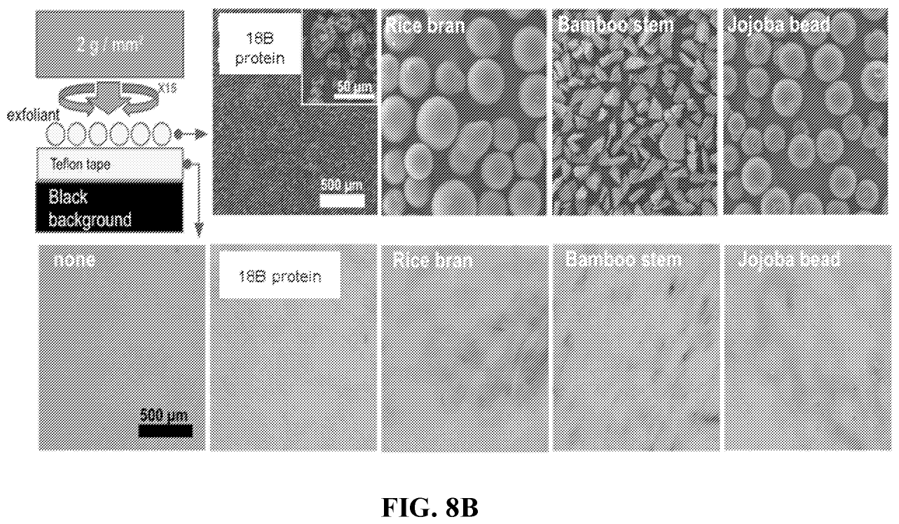

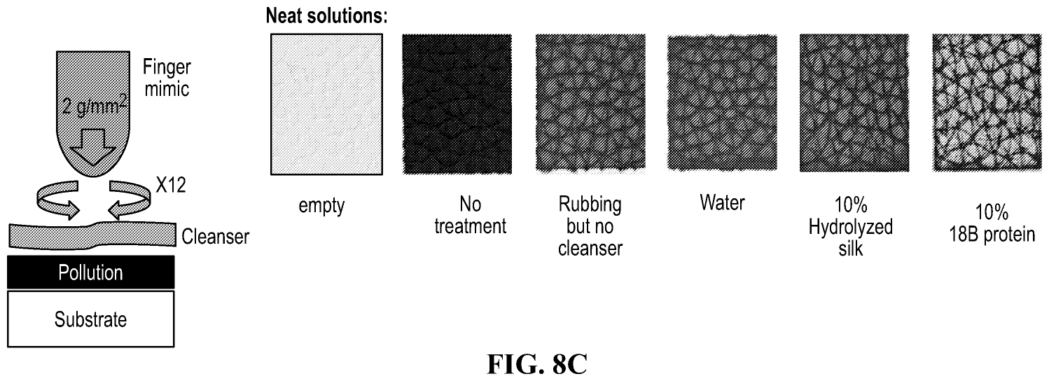

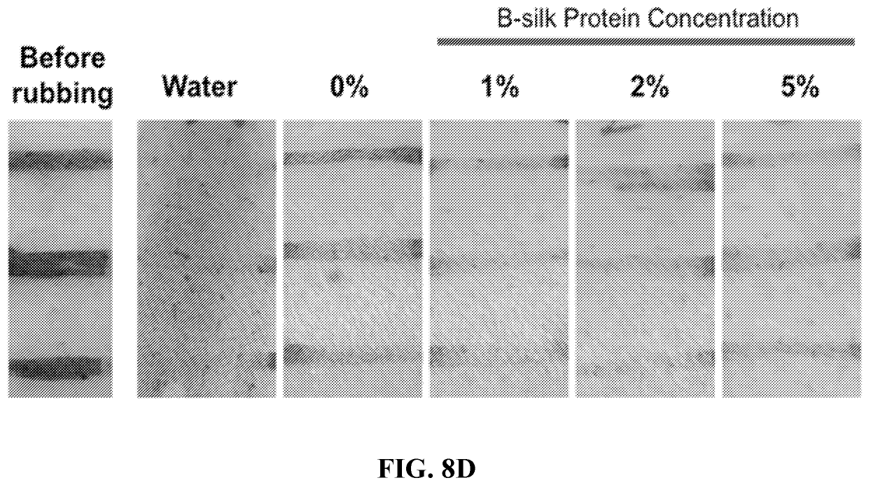

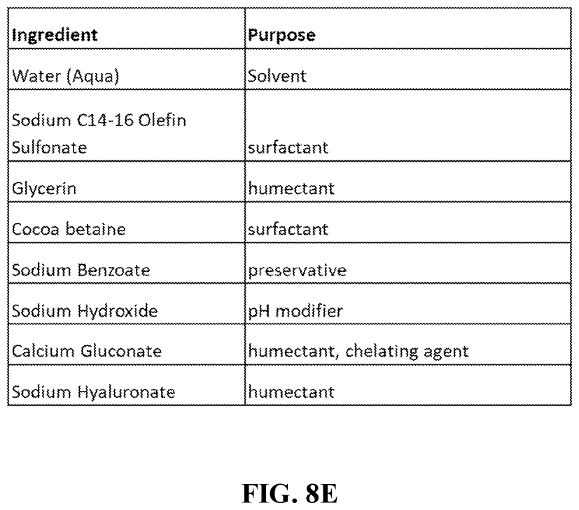

To investigate the cleansing effects of 18B powder, as produced according to the methods described in Example 1, a study was performed using black eyeshadow (Lancome), 18B powder, and standard cleansing agents. Black eyeshadow (Lancome) was applied evenly to the forearm area. A ⅜ teaspoon of exfoliant including 18B powder, charcoal black, and rice bran, was applied to the eye shadow. The area was rubbed with the ring finger 20 times in clockwise and counterclockwise motions. The sample labeled “No exfoliant” was also rubbed without any exfoliant, while the “untreated” sample was not rubbed. Each of the areas were then rinsed with tap water for 45 seconds. FIG. 8A shows various dry substances including 18B powder, charcoal black, and rice bran, rubbed on the skin over black eyeshadow and images after a water rinse. When rubbed on the skin in a dry state, 18B powder effectively removed make-up (e.g. black eyeshadow) compared to standard ingredients like charcoal black and rice bran.

To investigate the softness of 18B as a cleanser, the exfoliants were placed between a 2 g/mm² flat surface and a white Teflon tape so that the exfoliant entirely covered the surface of the Teflon tape (the surface of the Teflon tape was 1 cm in diameter and the amount of exfoliant used was approximately ¼ teaspoon), which covered a black background. Since Teflon is very soft, it stretched and thinned when it was exposed to something hard like an exfoliant, thereby exposing the black background. The surface of the Teflon tape was imaged with a light microscope in reflectance mode. FIG. 8B shows microscopic images of 18B powder used as an exfoliant on a skin substitute, as compared to a control and other standard ingredients including rice bran, bamboo stems, and jojoba beads. 18B powder was unique in that it effectively cleansed while also being incredibly soft. 18B powder was much less abrasive than standard ingredients as determined by much less black showing through the white Teflon.

To investigate use of 18B powder solutions as a cleansing solution, neat solutions were prepared by adding 18B powder to DI water. A 2 cm² area of faux leather (polyurethane material) was coated with carbon particles (5 μm in diameter). Excess particles were swept away. 200 μl of the solution was applied to the area and rubbed 14 times total in all four orthogonal directions. The samples were rinsed under flowing DI water for 45 seconds and patted dry. FIG. 8C shows a 10 wt % 18B powder solution used as a cleanser on a skin substitute, as compared to a control and hydrolyzed silk solutions. Specifically, the conditions tested included a negative (no pollution added) and positive control (pollution added with no cleansing or rubbing), water, rubbing but no cleanser, a 10 wt % hydrolyzed silk solution, and a 10 wt % 18B powder solution. Noticeably, compared to hydrolyzed silk, 18B powder more effectively removed pollution from the skin substitute.

To investigate cleansing effects of 18B powder in a base gel cleanser formulation, black eyeliner (Sephora) was applied to the forearm and dried for 10 minutes. The forearm was held under tap water for 45 seconds. 100 μl of the test article was applied to a 1 cm wide area over the eyeliner. The test article was rubbed up and down for a total of 14 times. Finally, the samples were rinsed under flowing DI water for 45 seconds. FIG. 8D shows various concentrations of an 18B powder solution used as cleansers, as compared to water without 18B powder. Specifically, 18B powder improved cleansing even after being added to a base gel cleanser formulation. In addition, the cleansing effectiveness did not necessarily scale with silk concentration. The 18B powder solutions used in this example varied in concentrations of 0 wt %, 1 wt %, 2 wt %, and 5 wt %. FIG. 8E shows the ingredient list for the 18B powder gel cleanser used in this example.

### Example 8: Anti-Aging Effects of Recombinant 18B Polypeptide Powder

To explore the anti-aging effects of 18B powder, as produced according to the methods described in Example 1, a clinical trial was performed. This study involved a third-party, double-blind, vehicle-controlled clinical trial (n=33) using expert grading, instrumental evaluation, and a subjective panelist questionnaire, as described further below. A 2 wt % 18B powder formulation was compared to a vehicle-only control. The formulation used for this study contained the following ingredients in addition to the 2 wt % 18B silk ingredient: water, caprylic/capric triglyceride, olive oil glycereth-8 esters, glycerin, coconut alkanes, methyl gluceth-20, Hydroxyethyl acrylate/sodium acryloyldimethyl taurate copolymer, tocopherol, dipotassium glycyrrhizate, coco-caprylate/caprate, pentylene glycol, chlorphenesin, caprylyl glycol, disodium EDTA, phenoxyethanol.

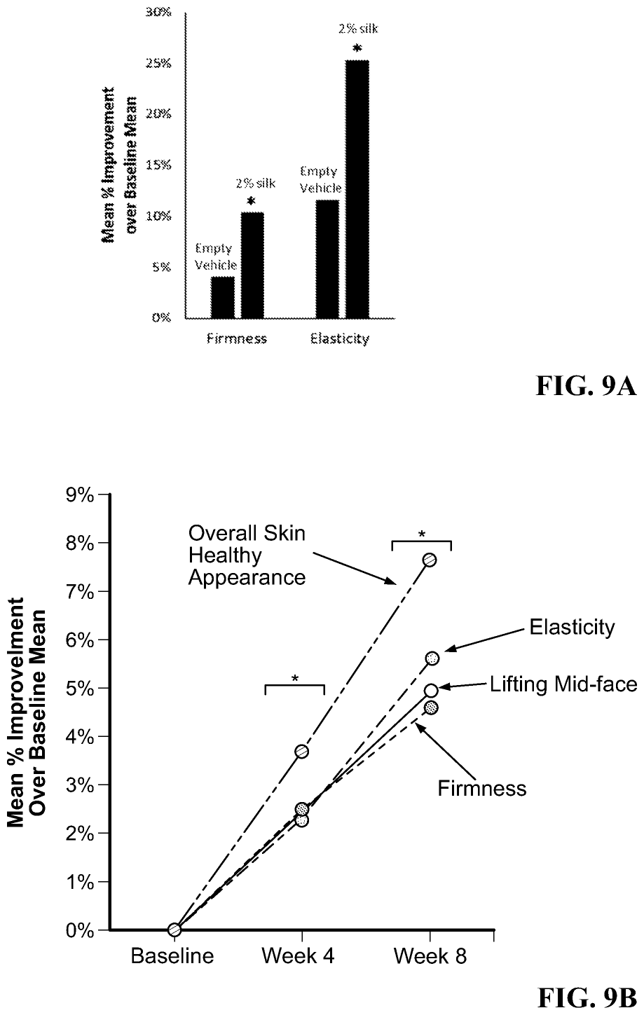

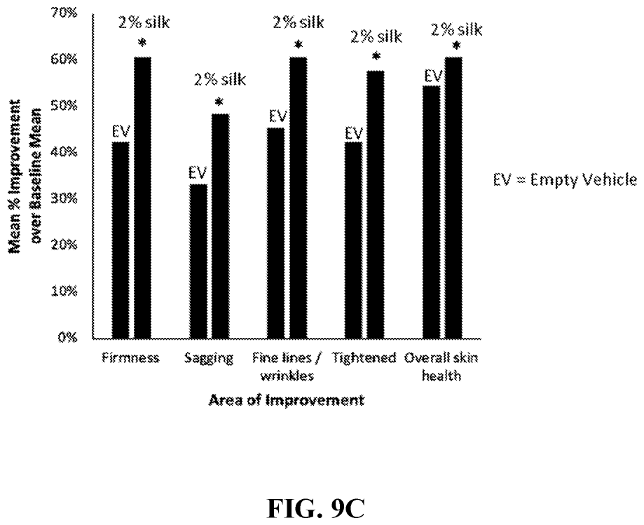

FIG. 9A shows a graph of mean percent improvement of 2 wt % 18B powder solution for firmness and elasticity of the skin. *=p<0.05 of 2 wt % sample compared to baseline measurement. Instrumental measurements using Cutometer measuring R0 for firmness and R5 for elasticity, showed statistical improvements over baseline at 12 weeks. R0 was a parameter that represented the passive behavior of the skin to force. R5 was a parameter that represented net elasticity. Skin firmness showed a 10% mean improvement over baseline for the 2 wt % 18B powder formulation. Skin elasticity showed a 25% mean improvement over baseline or the 2 wt % 18B powder formulation. The vehicle control did not show a statistical improvement over baseline. *=p<0.05 of 2 wt % 18B powder solution compared to baseline measurement.

FIG. 9B shows a graph of statistical improvement of 2 wt % 18B powder solution for lifting mid-face, elasticity, firmness, and overall skin healthy appearance over a period of 8 weeks. Expert grading showed statistical improvement compared to the empty vehicle control in lifting-mid-face, firmness, elasticity, and overall skin/healthy appearance. *=p<0.05 of 2 wt % sample compared to baseline measurement.

FIG. 9C shows skin results for a subjective panelist questionnaire after subjects used a 2 wt % 18B powder solution for 4 weeks. The subjective panelist questionnaire showed statistical improvement compared to the empty vehicle control at 4 weeks in the areas of firmness, sagging, fine lines and wrinkles, tightened skin, and overall health of skin. In the areas of firmness, sagging, fine lines and wrinkles, and tightened skin, there was about a 20% mean improvement. In the area of overall health of skin, there was about a 10% mean improvement.

This study was a 12-week, double-blind, vehicle controlled monadic evaluation of two facial skin treatments, a simple skin cream formulation (“empty vehicle”), and a simple skin cream formulation with 2 wt % silk content (“2% silk formulation”). The panel size was 33 people per sample. For the empty vehicle, the mean age was 59+/−6 years, and Fitzpatrick skin types were II, III, IV, and V. For the 2% silk formulation, the mean age was 58+/−6 years, and Fitzpatrick skin types were I, II, III, IV, and V. Instrumental assessments including Cutometer (MPA 580; Courage+Khazaka, Cologne Germany), on weeks 0 (“baseline”), 4, 8, and 12. The Cutometer MPA 580 (Courage+Khazaka, Germany) measured the viscoelastic properties of the skin by applying suction to the skin surface, drawing the skin into the aperture of the probe and determining the penetration depth using an optical measuring system. Skin elasticity was reported using the R5 (Ur/Ue) parameter, as the skin becomes more elastic, this value will increase. Skin firmness was reported using the R0 (Uf) parameter, as the skin becomes firmer this value will decrease. Clinical grading was performed at baseline and weeks 4, 8 and 12. All grading was performed in the same room for all subjects using overhead lighting as well as a lighted magnifying loop, as needed. Natural sunlight was blocked from the room to ensure the same lighting conditions at each time point. Visual Analog Scales (VAS) are commonly used in clinical research to measure intensity or frequency of various symptoms, subjective characteristics or attitudes that cannot be directly measured. VAS are a reliable scale and more sensitive to small changes than simple ordinal scales. When responding to a VAS item, the expert grader specified their level of agreement to a statement by indicating a position along a line (10 cm) between two end-points or anchor responses. Simple VAS were used to evaluate efficacy parameters in which the ends of a 10 cm horizontal line was defined as extreme limits orientated from the left (best) to the right (worst). Subjective questionnaires were used to gauge the subject's perception of the treatments and their effects on skin after 4, 8 and 12 weeks of treatment. Questions were asked for subjects' agreements to a statement with a five-point scale.

### Example 9: Wound Healing Effects of Recombinant 18B Polypeptide Powder

To assess wound healing effects of 18B powder, as produced according to the methods described in Example 1, wound scratch models were employed. Wound scratch model provided an in vitro qualitative estimation of the cell migration-inducing potential of a test material. In the first model, keratinocytes were used, which is the predominant type found in the epidermis of the skin. Normal neonatal human epidermal keratinocytes (HEK cat. #102-05n, Cell Applications, San Diego, CA) were grown in keratinocyte growth medium (KGM; optimal growth conditions) or in (10% KGM/90% DMEM; suboptimal growth conditions) in a 96 well plate to confluence. After, monolayers were scratched using a 10 μl pipette tip, and then were rinsed and incubated with the test material (18B powder diluted to 100 μg/mL in sterile distilled water) in duplicates for 48 hours. At the end of the experiment, cells were fixed in trichloroacetic acid and stained with sulforhodamine B. Microphotographs were taken with the EVOS 5000 imaging system (ThermoFisher Scientific, Waltham, MA). Scratch wound closure was analyzed with Celleste 5.0 software (Thermofisher).

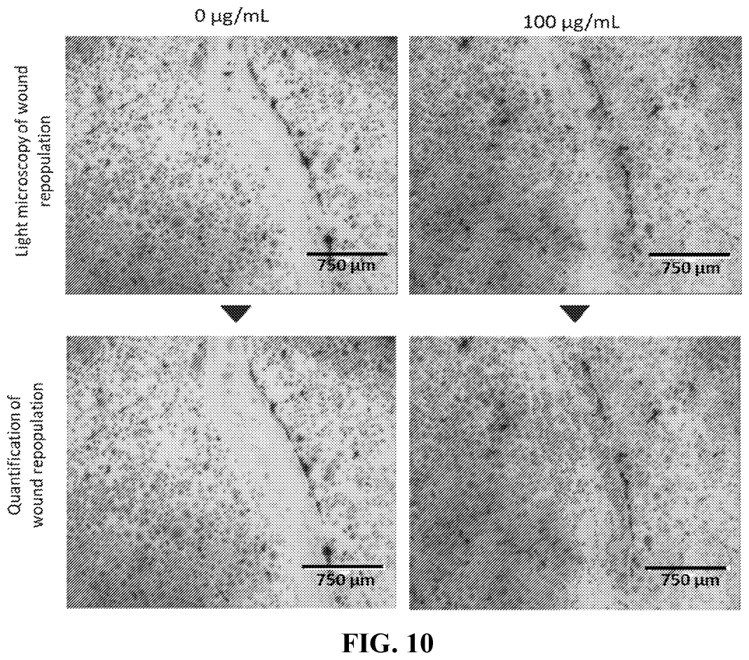

FIG. 10 shows light microscopy images of a keratinocyte wound scratch model 48 hours after the scratch was made and a computer-generated quantification of the wound closure after incubating cells with and without 100 μg/mL of 18B powder. The cells were incubated with and without 18B powder (100 μg/mL) and analyzed for extent of wound closure. Quantification of the wound closure showed an increased wound closure in the 18B powder-treated sample, indicated by the increased noise accumulation in the wound via computer generated quantification.

In the second model, fibroblasts were used. Normal neonatal human dermal fibroblasts (aHDF p. 4 cat. #106-05a, Cell Applications, San Diego, CA) were grown in DMEM (Invitrogen, Carlsbad, CA)+10% FBS (Sigma, St. Louis) and Pen/Strep/Fungizone solution (Lonza, Switzerland) in a 12 well plate to early subconfluence. The day of the experiment medium was changed to one with only 1 wt % fetal bovine serum, cell cultures were scratched using a 20 μl pipette tip, were rinsed and incubated with 18B powder diluted in sterile distilled water to 25 μg/mL and 50 μg/mL, in duplicates for 24 hours. Cells exposed to water and to 10 wt % fetal bovine serum were the negative and positive control, respectively. At the end of the experiment, cells were fixed in trichloroacetic acid and stained with sulforhodamine B. Microphotographs were taken with the EVOS 5000 imaging system (ThermoFisher Scientific, Waltham, MA). Scratch wound closure was analyzed with Celleste 5.0 software (Thermofisher).

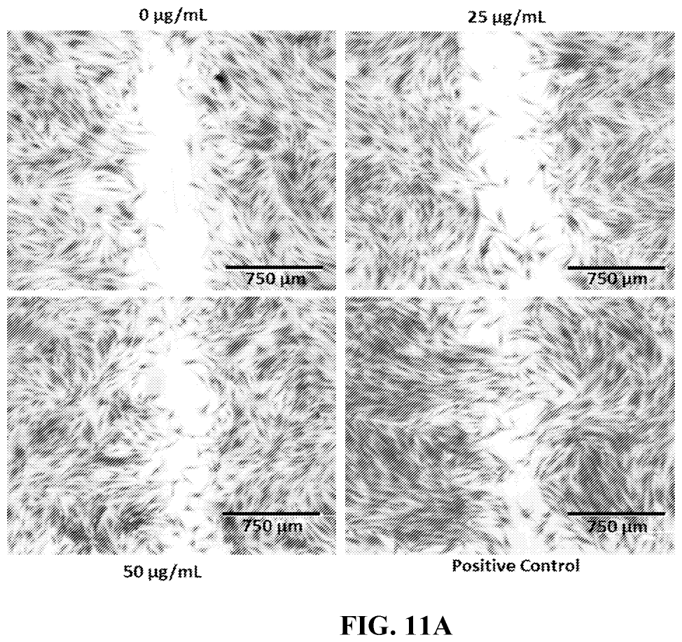

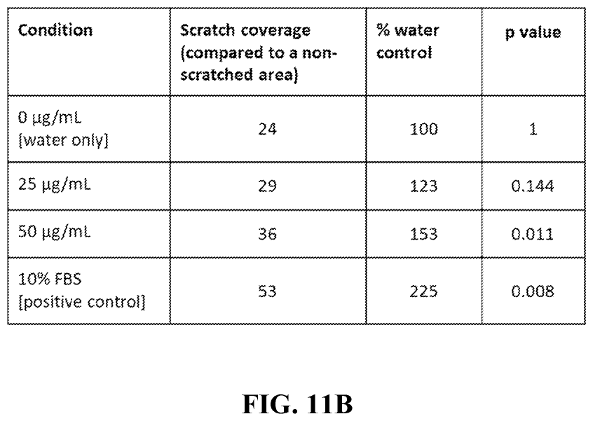

FIG. 11A shows light microscopy images of a fibroblast wound scratch model 24 hours after the scratch was made and quantification of the wound closure after incubating cells with and without various concentrations of 18B powder (25 μg/mL and 50 μg/mL), as compared to a positive control. The cells were incubated with and without 18B powder (25 μg/mL and 50 μg/mL) and analyzed for extent of wound closure.

FIG. 11B shows a quantification of the percent coverage of the wounded area by migrating fibroblasts after incubating cells with and without various concentrations of 18B powder (25 μg/mL and 50 μg/mL), as compared to a positive control, which yielded a scratch coverage of 53%, compared to water only at 24%. Quantification of the percentage of coverage of wounded area by migrating fibroblasts was performed with the Celleste software. The 50 μg/mL 18B powder sample showed about a 12% improved scratch coverage in the 18B powder-treated sample. A slight increase of 5% was measured in the 25 μg/mL 18B powder-treated sample. This model suggested a dose dependency in wound healing potential.

### Example 10: Additional Swelling Characteristics of Recombinant 18B Powder in Various Solvents

As described in Example 2, the powder particles exhibited differences in swelling depending on the solvent used to disperse the powder. Light microscopy images were taken by suspending powder in different solvents at 0.062% wt/wt levels. A droplet of the powder suspensions was loaded onto a glass slide and covered with a glass coverslip. Particle size data and light microscopy images were taken with the BeVision M1 particle analyzer equipped with a metallurgical microscope, programmable motorized stage, autofocus function, high-resolution CCD camera, and Bettersize particle sizing software. A circular scanning area was set with a radius of 0.5 cm using a 10× objective.

Deionized (DI at pH 6) water and PBS (Phosphate Buffered Saline at 7.2 pH) caused the powder to expand or swell similarly with 50% of particle diameters below 50 μm and max diameters at 118 μm and 116 μm, respectively. In contrast, pentylene glycol, silicone, and glycerin caused the particles to swell less with 100% of particle diameter below 75 μm. Olive oil exhibited a different behavior, with 50% of powder diameters below 30 μm; however, the max particle diameter was similar to that of particles in DI and PBS with 116 μm. These findings were compared to the dry, as-is powder in which 100% of the particle diameters were below 60 μm.

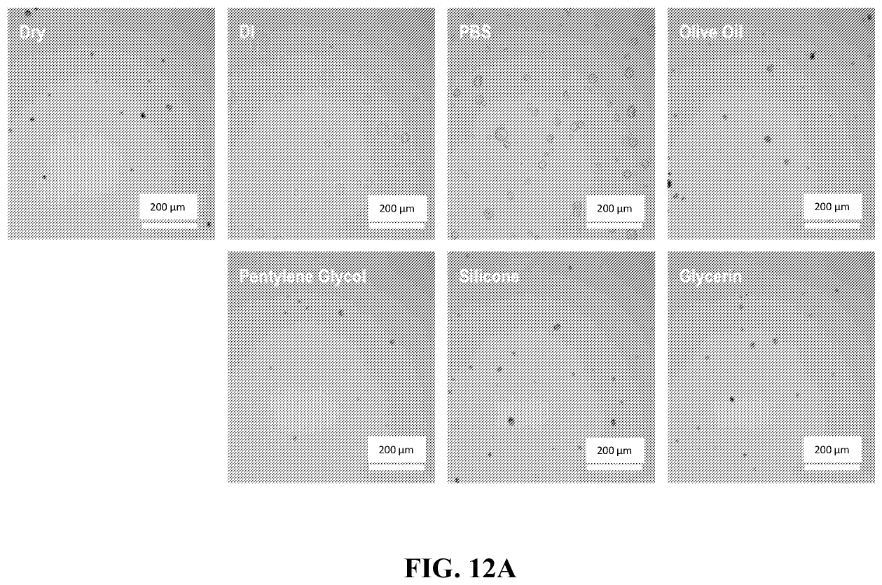

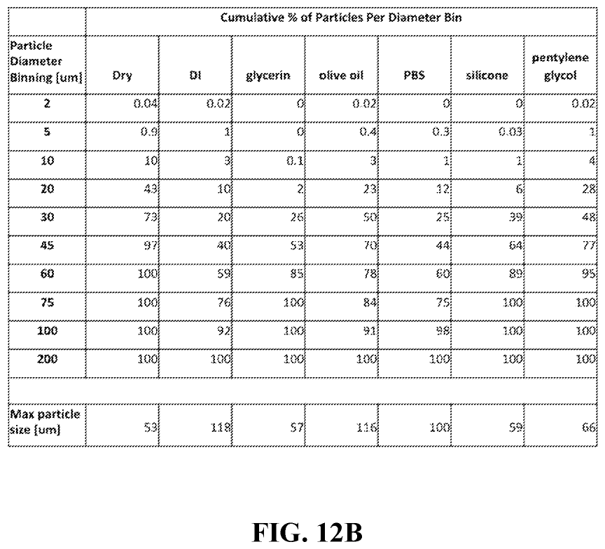

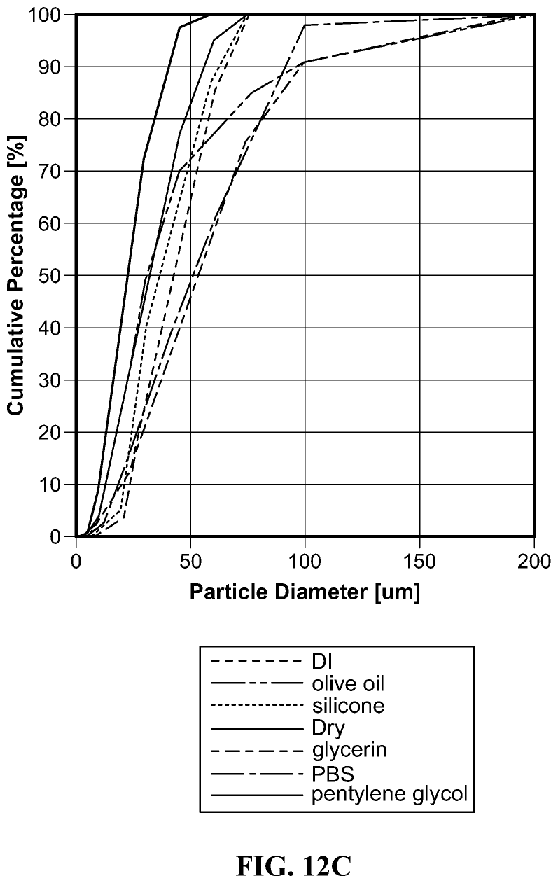

FIG. 12A shows representative powder morphology as viewed with light microscopy after resuspension in various different solvents used in beauty and personal care formulations.

FIG. 12B shows quantification of powder diameters in various solvents as determined by image analysis. Data was presented in tabular and graphical form as cumulative percentage of particles per diameter bin, as shown in FIG. 12C.

### Example 11: Solubility of Recombinant 18B Powder

Varying concentrations of recombinant 18B protein powder was dispersed in DI water at 1-10% wt. The solution was incubated for 24 hours at 22° C. The recombinant 18B powder was pelleted out with centrifugation at 16,000 RCF for 15 minutes at 4° C. The supernatant was poured off the pellet. Supernatant was dissolved in 5 M guanidine thiocyanate and injected onto a Yarra SEC-3000 SEC-HPLC column to separate constituents on the basis of molecular weight. Refractive index was used as the detection modality. The recombinant 18B (18B) aggregates, 18B monomer, low molecular weight (1-8 kDa) impurities, intermediate molecular weight impurities (8-50 kDa) and high molecular weight impurities (110-150 kDa) were quantified. Relevant composition was reported as mass percent. BSA was used as a general protein standard with the assumption that more than 90% of all proteins demonstrate dn/dc values, the response factor of refractive index, within about 7% of each other. Poly(ethylene oxide) was used as a retention time standard, and a BSA calibrator was used as a check standard to ensure consistent performance of the method.

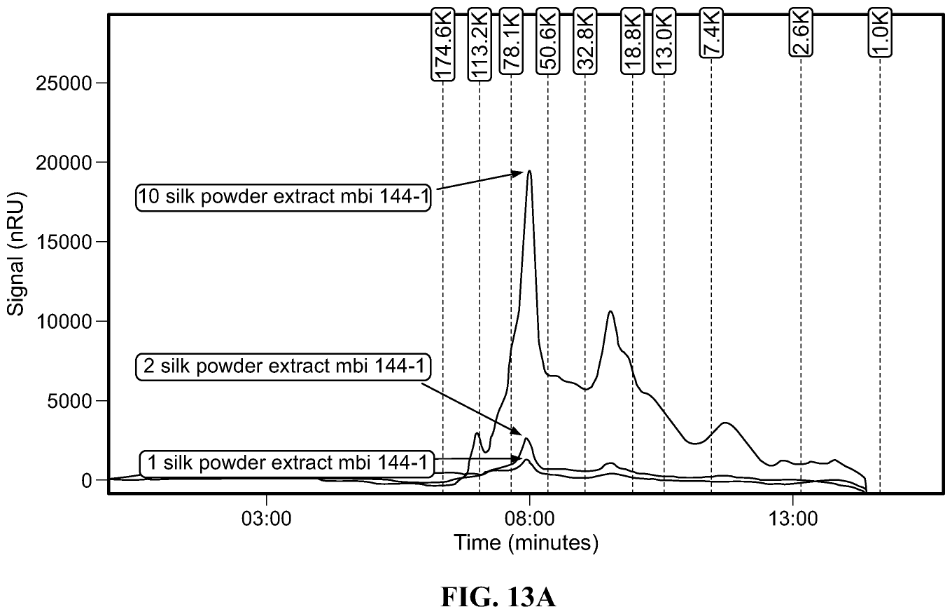

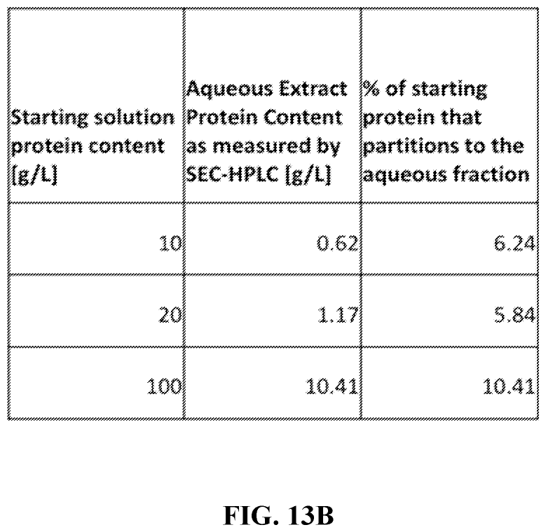

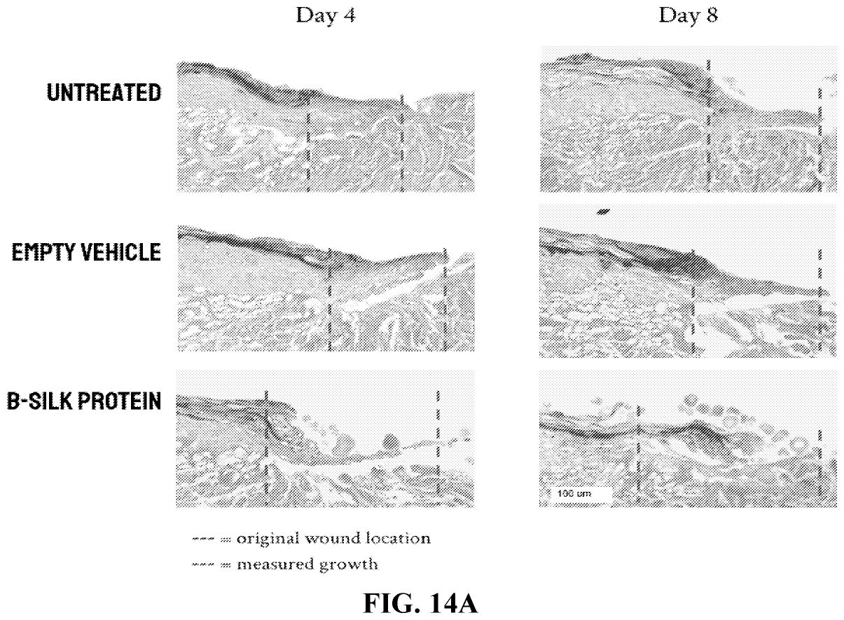

FIG. 13A shows quantification of the solubility of various recombinant 18B protein powder solutions as determined by size exclusion chromatography (SEC). Full-length 18B protein molecules were found in the region between 50.6 kDa and 78.1 kDa. 18B protein aggregates were found around 113.2 kDa and shorter length versions of the 18B protein were found between 2.6 kDa and 50.8 kDa. All of these protein species were used in the calculation for 18B protein solubility in the extract. The amount of the 18B protein measured in the aqueous extract scales with the solution concentration, indicating that no maximum solubility was reached. Note that 10% wt/wt 18B protein powder in DI water was roughly the upper limit of the suspension capability (i.e. above 10% wt/wt 18B protein in DI, the powder did not wet fully). FIG. 13B shows a table of the solubility results. The recombinant 18B protein powder exhibited limited solubility in DI water as determined by HPLC SEC. Less than 11% of the protein partitioned into the aqueous solvent.

### Example 12: Accelerated Wound Healing Effects of Recombinant 18B Powder

Human skin was obtained from an abdominoplasty obtained from a 41-year-old Caucasian woman with a phototype II based on the Fitzpatrick classification. A total of 21 human skin explants of an average diameter of 11 mm (±1 mm) and 21 rectangular skin explants of 10×15 mm size were prepared. The explants were kept in culture medium at 37° C. in a humid, 5% CO₂ atmosphere. On day 0, the mechanical wounds were performed in the center of each explant of the batches using a 2 mm diameter-punch. On day 0 after the wound, day 1, day 4 and day 6, the empty vehicle (PBS+0.9% Botanistat preservative) and the recombinant 18B protein sample (5% recombinant 18B protein in PBS+0.9% Botanistat preservative) or the empty vehicle sample (PBS+0.9% Botanistat preservative) were applied topically based on a surface area of 2 mg/cm² and were spread using a small spatula. The untreated control explants did not receive any treatment, except for the renewal of the culture medium. Half of the culture medium (1 ml) was renewed on day 1, day 4 and day 6. On day 0, three explants from each treatment condition were collected and cut in two parts. Half of the samples were fixed in buffered formalin solution, and half were frozen at −80° C. On day 4 and day 8, three explants from each batch were collected and processed in the same way. After fixation for 24 hours in buffered formalin, the samples were dehydrated and impregnated in paraffin using a Leica PEARL dehydration automat. The samples were then embedded using a Leica EG 1160 embedding station. 5 μm-thick sections were made using a Leica RM 2125 Minot-type microtome, and the sections were then mounted on Superfrost® histological glass slides.

The frozen samples were cut into 7-μm-thick sections using a Leica CM 3050 cryostat. Sections were then mounted on Superfrost® plus silanized glass slides. The microscopical observations were made using a Leica DMLB or Olympus BX43 microscope. Pictures were digitized with a numeric DP72 Olympus camera with CellSens storing software. Cell viability and wound closure measurements in the epidermal and dermal structures were performed after staining of paraffinized sections according to Masson's trichrome, Goldner variant.

As a result, recombinant 18B protein supported accelerated wound closure in an ex vivo human skin model by increasing cellular migration in the wound site. Human skin explants were wounded and treated with a recombinant 18B protein sample for 8 days.

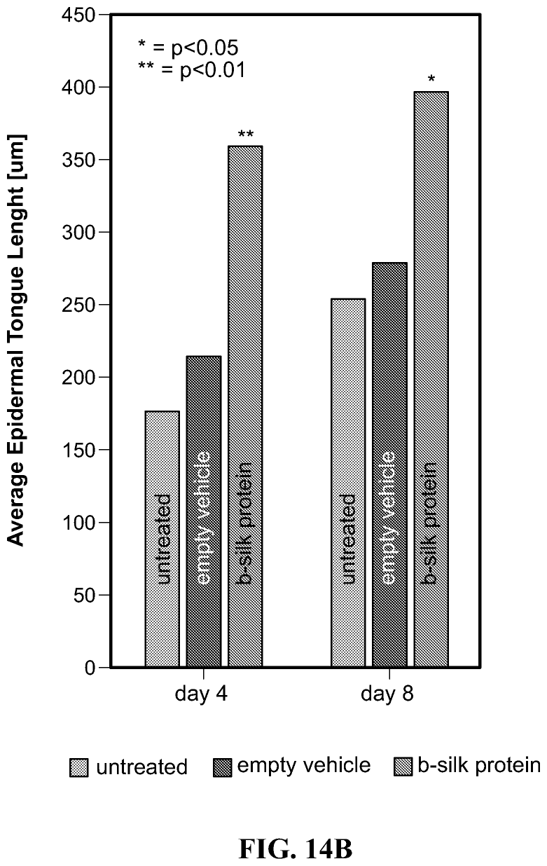

FIG. 14A shows histological cross-sections of the ex vivo tissues. It is shown that recombinant 18B protein outperformed the empty vehicle and untreated control for the extent of epidermal length at both day 4 and day 8 timepoints. FIG. 14B shows that recombinant 18B protein induced a 68% increase in epidermal tongue length on day 4 compared to the empty vehicle (**=p<0.01 compared to untreated and empty vehicle controls). On day 8, recombinant 18B protein induced a 42% increase in epidermal tongue length on day 8 compared to the empty vehicle (*=p<0.05 compared to untreated and empty vehicle controls).

### Example 13: Recombinant 18B Powder Reduces Basal Level of Oxidative Stress and Oxidative Stress Caused by Blue Light Irradiation

Twenty-seven (27) human skin explants of an average diameter of 12 mm (1 mm) were prepared on an abdominoplasty coming from a 52-year-old Caucasian woman with a phototype II based on the Fitzpatrick classification. The explants were kept in survival in culture medium at 37° C. in a humid, 5%—CO₂ atmosphere. The study was performed on the biopsies obtained from surgical residues after written informed consent from the donor. On day 0, day 1, and day 4 thirty minutes before blue light irradiation, the recombinant 18B protein sample (2% recombinant 18B protein in PBS+0.9% Botanistat preservative) or the empty vehicle sample (PBS+0.9% Botanistat preservative) were applied topically on the basis of a surface area of 2 mg/cm² and were spread using a small spatula. The untreated control explants did not receive any treatment except the renewal of culture medium. The culture medium was half renewed (1 mL per well) on day 1 and day 4. On day 4, the explants were slated for blue-light irradiation, put in 1 ml of HBSS (Hanks' balanced salt solution) and irradiated with blue light using the Solarbox® device for 3 hours at a dose of 63.75 J/cm². The untreated, non-irradiated control explants were kept in 1 mL of HBSS in darkness during the irradiation process. At the end of the irradiation, all batches were put back in culture medium. When samples were ready to be sacrificed, they were collected and cut in two parts. Half of the samples were fixed in buffered formalin and half were frozen at −80° C. After fixation for 24 hours in buffered formalin, the samples were dehydrated and impregnated in paraffin using a Leica PEARL dehydration automat. The samples were embedded using a Leica EG 1160 embedding station. 5 m-thick sections were made using a Leica RM 2125 Minot-type microtome, and the sections were mounted on Superfrost® histological glass slides. The microscopical observations were realized using a Leica DMLB, Olympus BX43 or an Olympus BX63 microscope. Pictures were digitized with a numeric DP72 or DP74 Olympus camera with celSens storing software. The cell viability of epidermal and dermal structures was observed on formol-fixed paraffin-embedded (FFPE) skin sections after Masson's trichrom staining, Goldner variant. The cell viability was assessed by microscopical observation. 8-OHdG immunostaining was performed on FFPE skin sections with a monoclonal anti-8-OHdG antibody (Gentaur, ref. 50-MOG, clone N45-1) diluted at 1:400 in PBS—BSA 0.3% and incubated overnight at room temperature using a Vectastain Kit Vector amplifier system avidin/biotin, and revealed by VIP, a substrate of peroxidase (Vector laboratories, Ref. SK-4600) giving a violet staining once oxidized. The immunostaining was performed manually, assessed by microscopical observation and semi-quantified by image analysis.

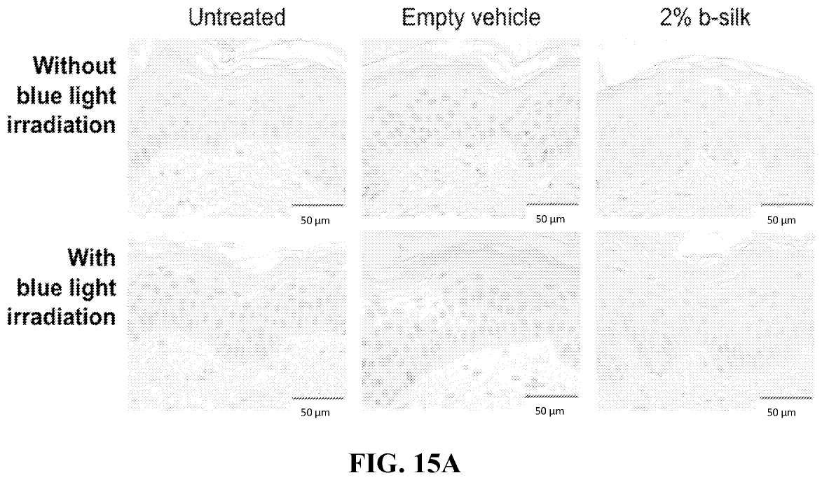

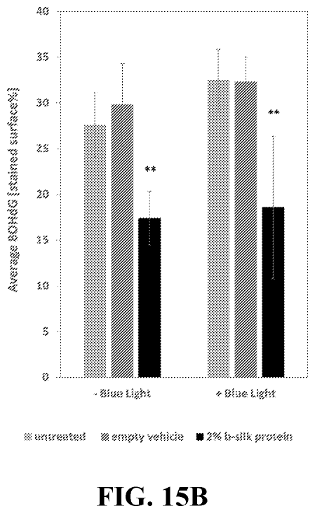

The semi-quantified image analysis was performed as follows: first, the stain was detected and the pixels that corresponded to the staining were selected—this was assigned to mask 1. Then, the selection of the ROI (i.e. the epidermal layer) was selected by drawing and assigned to another mask 2. Next, the overlap of masks (i.e. where the immunostaining and ROI overlap) was assigned to mask 3. Finally, the percentage of the epidermis (i.e. mask 2) covered by the staining (i.e. mask 3) was calculated and termed “stained surface %”. FIG. 15A shows that recombinant 18B protein reduced basal level of oxidative stress as measured by a decrease of a marker of nuclear oxidation (8-OHdG). Moreover, recombinant 18B protein also reduced blue light-induced nuclear oxidation (8-OHdG). Nuclear and mitochondrial DNA oxidation occurred most readily at guanine residues due to the high ionization potential of this base. 8-oxo-2′-deoxyguanosine (8-oxo-dG) or hydroxydesoxyguanosine (8-OHdG) is one of the predominant forms of free radical-induced oxidative lesions in humans. The interaction of hydroxyl radicals with the double bond at the C-8 position of the guanine base leads to the production of 8-OHdG. This stable oxidative modified DNA product has extensively been used to reflect the degree of oxidative damage to DNA.

FIG. 15B shows that a simple solution of 2% recombinant 18B protein (suspended in PBS and 0.9% Botanistat preservative) resulted in a 37% decrease in 8-OHdG staining compared to untreated sample and 42% compared to the empty vehicle (p<0.01). Blue irradiations induced an 18% increase in 8-OHdG staining (p<0.01) of the untreated control, while the sample treated with 2% recombinant 18B protein solution experienced a 43% decrease in 8-OHdG staining compared to blue light irradiated untreated control and 42% compared to the blue light irradiated empty vehicle (p<0.01).

### Example 14: Recombinant 18B Powder Attenuates Pollution Induced Oxidative Stress

Thirty-five (35) human skin explants of an average diameter of 12 mm (±1 mm) were prepared on an abdominoplasty coming from a 59-year-old Caucasian woman with phototype II-III based on the Fitzpatrick classification. The explants were kept in survival in culture medium at 37° C. in a humid, 5%—CO₂ atmosphere. The study was performed on biopsy obtained from surgical residues after written informed consent from the donor.

On day 0, day 3, and day 4 before pollutant exposure, the recombinant 18B protein sample (2% recombinant 18B protein in PBS+0.9% Botanistat preservative) or the empty vehicle sample (PBS+0.9% Botanistat preservative) were applied topically based on a surface area of 2 mg/cm² and spread using a small spatula. The untreated control explants did not receive any treatment except the renewal of culture medium. The culture medium was half renewed (1 mL per well) on day 1 and completely renewed (2 mL per well) on day 4 after pollutant exposure. On day 4, the explants slated for pollution exposure were placed on the PolluBox® system with 900 μl per well of HBSS, and were exposed by spraying a mixture of polycyclic aromatic hydrocarbons+heavy metals with 0.9% NaCl (150 μl of NaCl 0.9% per ml of pollutant solution) for 1.5 hours using 3 mL total of the entire pollution solution. The untreated control explants were kept in 1 mL of HBSS. At the end of the pollutant exposure, all explants were put back into 2 mL of fresh culture medium. When samples were ready to be sacrificed, they were collected and cut in three parts. One part was fixed in buffered formalin, the second part was frozen at −80° C., and the last part was put in RNA Later®. After fixation for 24 hours in buffered formalin, the samples were dehydrated and impregnated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The frozen samples were cut into 7-μm-thick sections using a Leica CM 3050 cryostat. Sections were then mounted on Superfrost® plus silanized glass slides. The microscopical observations were realized using a Leica DMLB, Olympus BX43 or an Olympus BX63 microscope. Pictures were digitized with a numeric DP72 or DP74 Olympus camera with celSens storing software. The cell viability of epidermal and dermal structures was observed on formol-fixed paraffin-embedded (FFPE) skin sections after Masson's trichrome staining, Goldner variant. The cell viability was assessed by microscopical observation. Nrf2 immunostaining was performed on FFPE skin sections with a monoclonal anti-phospho (S40) Nrf2 antibody (Abcam, ref. ab76026, clone EP1809Y) diluted at 1:400 in PBS—BSA 0.3%-Tween 20 (0.05%), for one hour at room temperature, using Vectastain Kit Vector amplifier system avidin/biotin, and revealed by VIP, a violet substrate of peroxidase (Vector Laboratories, ref. SK-4600).

The immunostaining was assessed by microscopical observation. IL-1α immunostaining was performed on FFPE skin sections with a monoclonal anti-IL-1α antibody (Novus Biologicals, NBP2-45400, clone OTI2F8) diluted at 1:200 in PBS—BSA 0.3%-Tween 20 (0.05%), for one hour at room temperature, using Vectastain Kit Vector amplifying system avidin/biotin, and revealed by VIP, a violet substrate of peroxidase (Vector Laboratories, ref SK-4600).

The immunostaining was assessed by microscopical observation. The immunostaining was performed manually, assessed by microscopical observation and semi-quantified by image analysis. The semi-quantified by image analysis was performed as follows: first, the stain was detected and the pixels that corresponded to the staining were selected—this was assigned to mask 1. Then, the selection of the ROI (i.e. the epidermal layer) was selected by drawing and assigned to another mask 2. Next, the overlap of masks (i.e. where the immunostaining and ROI overlap) was assigned to mask 3. Finally, the percentage of the epidermis (i.e. mask 2) covered by the staining (i.e. mask 3) was calculated and termed “stained surface %”.

As a result, recombinant 18B protein reduced pollution-induced expression of cellular antioxidation systems and inflammation. Under oxidative stress, cellular antioxidation systems are triggered. Namely, the nuclear factor erythroid 2-related factor 2 (Nrf2) is activated by phosphorylation and translocates from the cytoplasm to the nucleus. Once in the nucleus, Nrf2 binds to the DNA at the location of the Antioxidant Response Element (ARE) or Human Antioxidant Response Element (hARE), which is the master regulator of the total antioxidant system. Also, under oxidative stress, inflammation cascades are triggered. In the epidermis, the interleukins 1 (IL-1) is able to modulate keratinocyte proliferation, immune and anti-microbial responses, inflammation and lipid synthesis. In general, IL-1 is responsible for the production of inflammation, as well as the promotion of fever and sepsis. In this study, IL-la was studied.

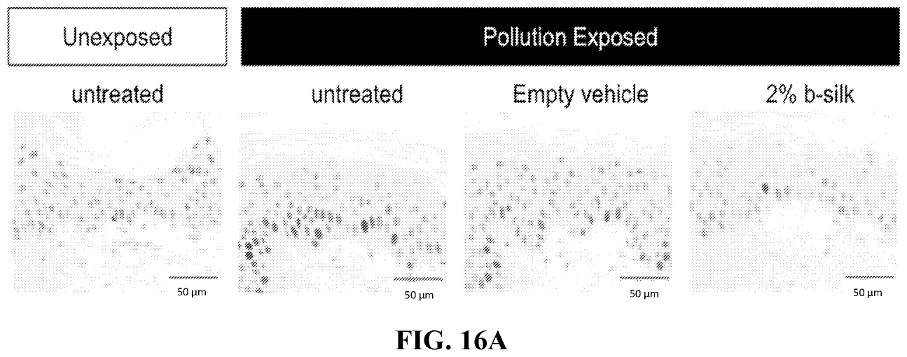

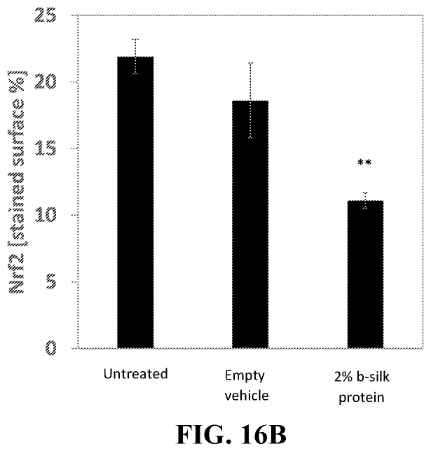

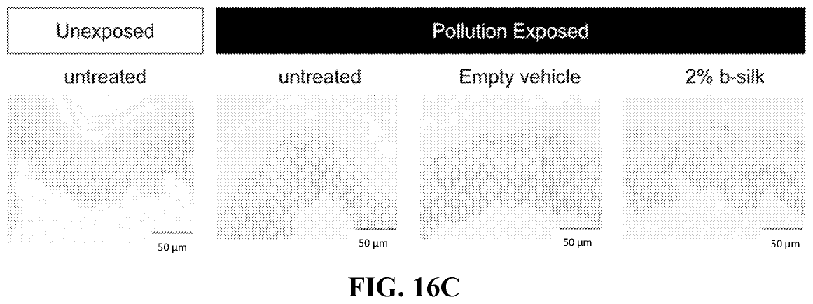

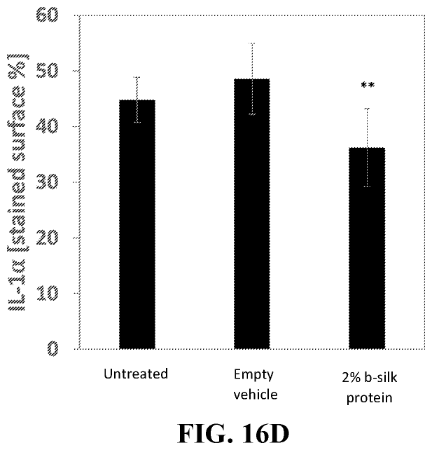

FIG. 16A shows histological cross-sections of ex-vivo tissues of untreated, empty vehicle, and 2% recombinant 18B protein samples with and without exposure to pollution. FIG. 16B shows that a solution of 2% recombinant 18B protein suspended in PBS and 0.9% Botanistat preservative resulted in a significant decrease in Nrf2 expression (stained surface %) when exposed to pollution, compared to the untreated (49% less, p<0.01) and the empty vehicle (40% less, **p<0.01) samples. Note that the untreated sample exposed to pollution exhibited a significant increase of 68% compared to the untreated, unexposed sample (p<0.01). Also, note that the empty vehicle decreased Nrf2 expression when exposed to pollution compared to the untreated (15% less, p<0.01) sample but to a much lesser degree than the 2% recombinant 18B protein samples.

FIG. 16C shows histological cross-sections of ex-vivo tissues of untreated, empty vehicle, and 2% recombinant 18B protein samples with and without exposure to pollution. FIG. 16D shows that compared to the empty vehicle, the 2% recombinant 18B samples induced a significant decrease in IL-la expression when exposed to pollution compared to the untreated (19% less, p<0.01) and the empty vehicle (26% less, **p<0.01) samples. Note that the untreated sample exposed to pollution exhibited a significant increase of 47% compared to the untreated, unexposed sample (p<0.01).

### Example 15: Recombinant 18B Powder Attenuates UVA/UVB Induced Oxidative Stress

Thirty (30) human skin explants of an average diameter of 11 mm (±1 mm) were prepared on an abdominoplasty coming from a 52-year-old Caucasian woman with phenotype II based on the Fitzpatrick classification. The explants were kept in survival in BEM culture medium (BIO-EC's Explants Medium) at 37° C. in a humid, 5%—CO₂ atmosphere. On day 0, day 1, and day 4 before UV irradiations, the recombinant 18B protein sample (2% recombinant 18B protein in PBS+0.9% Botanistat preservative) or the empty vehicle sample (PBS+0.9% Botanistat preservative) were applied topically based on a surface area of 2 mg/cm² and spread using a small spatula. The untreated control explants did not receive any treatment except the renewal of culture medium. The culture medium was half renewed (1 mL per well) on day 1 and completely renewed (2 mL per well) on day 4. On day 4, the “UVA” batches were irradiated using a UV simulator Vibert Lourmat RMX 3W with a dose of 18 J/cm² of UVA corresponding to 4 MED (minimal erythemal dose). The “UVB” batches were irradiated using a UV simulator Vibert Lourmat RMX 3W with a dose of 0.3 J/cm² of UVB corresponding to 2 MED (minimal erythemal dose). The unirradiated batches were kept in HBSS in the dark. At the end of the irradiation, all the explants were put back in 2 mL of culture medium. When samples were ready to be sacrificed, they were collected and cut in two parts. One part was fixed in buffered formalin, the second part was frozen at −80° C. After fixation for 24 hours in buffered formalin, the samples were dehydrated and impregnated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The frozen samples were cut into 7-μm-thick sections using a Leica CM 3050 cryostat. Sections were then mounted on Superfrost® plus silanized glass slides. The microscopical observations were realized using a Leica DMLB, Olympus BX43 or an Olympus BX63 microscope. Pictures were digitized with a numeric DP72 or DP74 Olympus camera with celSens storing software. The cell viability of epidermal and dermal structures was observed on formol-fixed paraffin-embedded (FFPE) skin sections after Masson's trichrome staining, Goldner variant. Nrf2 immunostaining was performed on FFPE skin sections with a monoclonal anti-phospho (S40) Nrf2 antibody (Abcam, ref. ab76026, clone EP1809Y) diluted at 1:400 in PBS—BSA 0.3%-Tween 20 (0.05%), for one hour at room temperature, using Vectastain Kit Vector amplifier system avidin/biotin, and revealed by VIP, a violet substrate of peroxidase (Vector Laboratories, ref. SK-4600). The immunostaining was performed using an automated slide processing system (Autostainer, Dako) and assessed by microscopical observation. Thymine dimers immunostaining was performed on FFPE skin sections with a monoclonal anti-thymine dimers antibody (Kamiya, ref. MC-062, clone KTM53) diluted at 1:1600 in PBS—BSA 0.3%-Tween 20 at 0.05% and incubated 1 hour at room temperature using a Vectastain Kit Vector amplifier system avidin/biotin, and revealed by VIP, a substrate of peroxidase (Vector laboratories, Ref. SK-4600) giving a violet signal once oxidized. The immunostaining was performed manually, assessed by microscopical observation and semi-quantified by image analysis.

The semi-quantified by image analysis was performed as follows: first, the stain was detected and the pixels that corresponded to the staining were selected—this was assigned to mask 1. Then, the selection of the ROI (i.e. the epidermal layer) was selected by drawing and assigned to another mask 2. Next, the overlap of masks (i.e. where the immunostaining and ROI overlap) was assigned to mask 3. Finally, the percentage of the epidermis (i.e. mask 2) covered by the staining (i.e. mask 3) was calculated and termed “stained surface %.” For quantifying the number of sunburned cells, the histology was counted for the number of cells with eosinophilic cytoplasm and an apoptotic nucleus.

As a result, recombinant 18B protein attenuated the negative effects associated with UVA/UVB exposure to the epidermis, namely cell viability, thymine dimer expression, and Nrf2 expression. Ultraviolet light is absorbed by a double bond in thymine and cytosine bases in DNA. This added energy opens the bond and allows it to react with a neighboring base. If the neighbor is another thymine or cytosine base, it can form a covalent bond between the two bases.

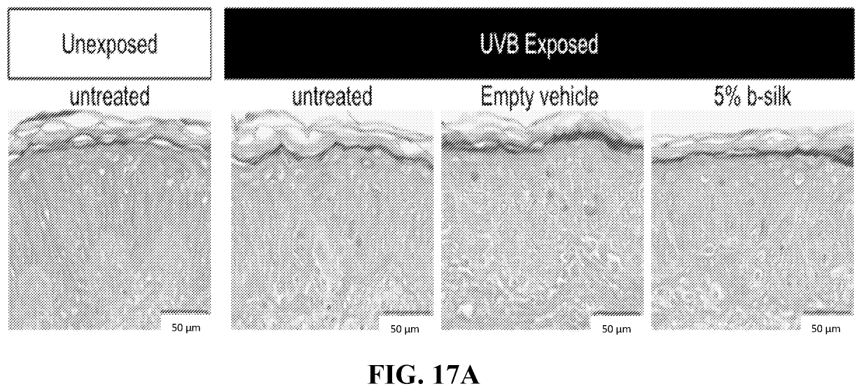

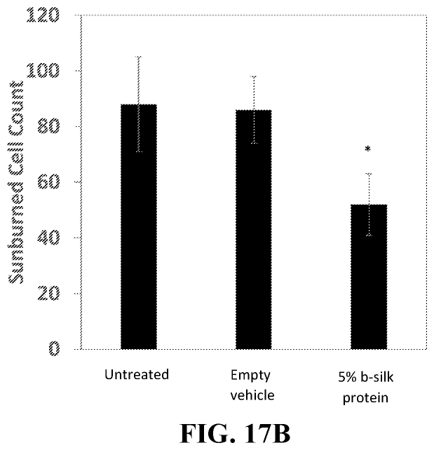

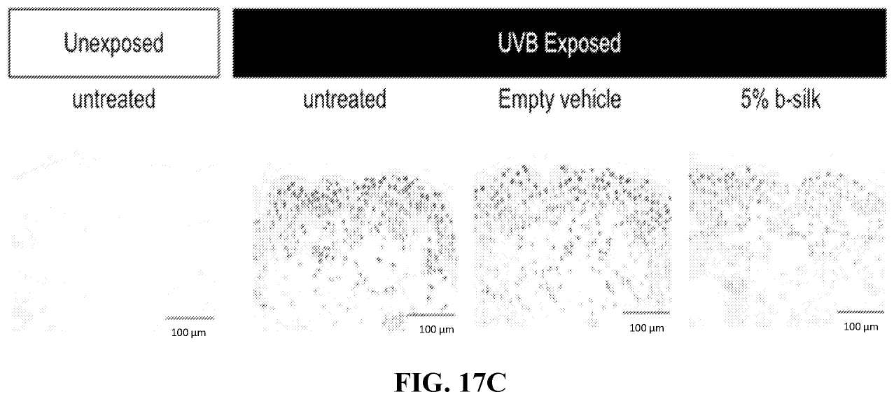

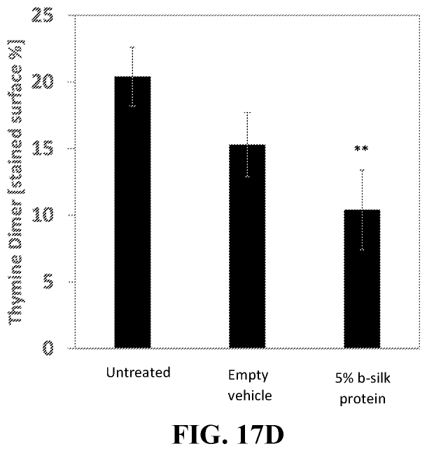

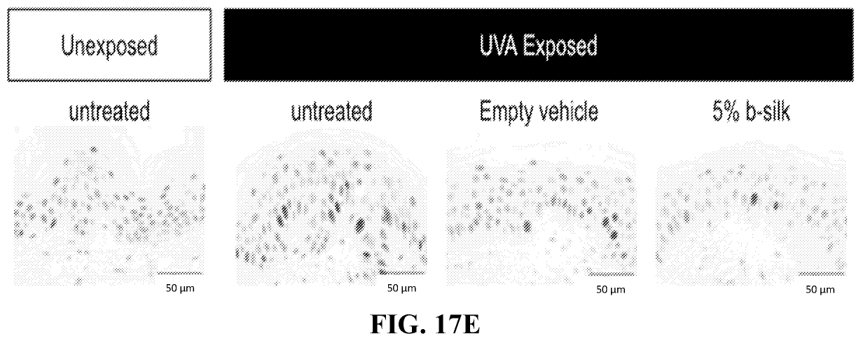

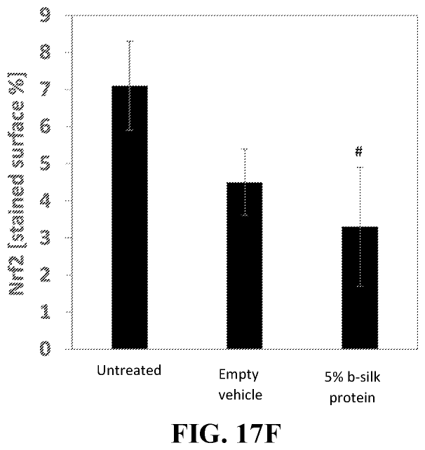

FIG. 17A shows histological cross-sections of ex-vivo tissues of untreated, empty vehicle, and 5% recombinant 18B protein samples with and without exposure to UVB. FIG. 17B shows that 5% recombinant 18B protein (suspended in PBS and 0.9% Botanistat preservative) resulted in fairly favorable cell viability when exposed to UVB compared to the untreated and empty vehicle samples. The recombinant 18B protein samples reduced the number of sunburned cells compared to the untreated (40% less, *=p<0.05) and empty vehicle controls (39% less, *=p<0.05); recombinant 18B protein slightly protected against the alterations induced by UVB exposure.

FIG. 17C shows histological cross-sections of ex-vivo tissues of untreated, empty vehicle, and 5% recombinant 18B protein samples with and without exposure to UVB. FIG. 17D shows that 5% recombinant 18B protein (suspended in PBS and 0.9% Botanistat preservative) resulted in attenuated expression of thymine dimers compared to the untreated and empty vehicle controls. The 5% recombinant 18B protein induced a significant decrease in thymine dimers expression of 49% compared to the untreated control and 32% compared to the empty vehicle (**=p<0.01). The untreated sample irradiated with UVB exhibited 20.4% of epidermis surface positive to thymine dimers immunostaining, compared to the untreated, unirradiated sample which had no expression of thymine dimers.

FIG. 17E shows histological cross-sections of ex-vivo tissues of untreated, empty vehicle, and 5% recombinant 18B protein samples with and without exposure to UVA. FIG. 17F shows 5% recombinant 18B protein (suspended in PBS and 0.9% Botanistat preservative) resulted in attenuated expression of Nrf2 staining after exposure to UVA. The UVA irradiation induced a significant increase of the expression of activated Nrf2 in the epidermis, compared to the unexposed sample (40% more, p<0.01). 5% recombinant 18B protein exhibited a decrease in Nrf2 expression upon UVA exposure compared to the empty vehicle (26%, *=p<0.1) and untreated controls (53%, p<0.01). Also, the empty vehicle decreased Nrf2 staining when exposed to UVA compared to the untreated (36% less, p<0.01) samples, but to a smaller degree than the 2% recombinant 18B protein samples.

### Example 16: Mattifying Effects of Recombinant 18B Powder

In a 9-subject study, subjects experienced a greater increase from baseline in glossiness for the empty vehicle formulation compared to the 2% 18B protein formulation in two-thirds of the subjects.

In a pre-study visit potentially qualifying volunteers arrived with a clean face (no makeup) and were visually screened for oily foreheads and, if they appeared to qualify, they completed the consent process. Subjects consisted of women aged 18 to 65 years old (inclusive) with phenotype I-II based on the Fitzpatrick classification and moderate-to-severe sebum on the forehead as measured by Sebumeter®. Candidates were recruited from a pool of healthy women who meet the inclusion/exclusion criteria. The inclusion criteria were as follows: a) female ages 18-65 years; b) Fitzpatrick Skin Type I-II; c) was able to read, understand, and sign the informed consent form; d) had moderate-to-severe sebum on the forehead as measured by Sebumeter®; e) was willing to arrive at their PSV/DOT visit with a clean face (no makeup or topical products applied since their last wash); f) agreed to not wear a hat, wig, or other head covering to the visit and wore or be provided with a headband to keep their hair off of their forehead during the visit; g) was willing and able to follow all study requirements and restrictions.

Exclusion criteria were as follows: a) was pregnant, nursing, or planning a pregnancy, as determined by interview; b) had any known sensitivities or allergies to skin care products, cosmetics, moisturizers, sunscreens, fragrances, or any ingredients in the IPs; had any tattoos, marks, scars, scratches, moles, or other blemishes on the test sites that would interfere with the study; d) had a skin condition on the face other than oily skin (e.g., psoriasis, eczema, etc.); e) had a history of a confirmed or suspected COVID-19 infection within 30 days prior to the study visit; f) had contact with a COVID-19-infected person or persons within 14 days prior to the study visit; g) individual or a member of the individual's household had traveled internationally within 14 days prior to the study visit; h) had experienced any of the following self-reported symptoms of COVID-19 within 2 weeks prior to the study visit; i) was currently participating in another study or is scheduled to participate in another study during this study period; j) was an employee, contractor, or immediate family member of Dermico Lab, the Principal Investigator, or the study sponsor; j) other condition or factor the Principal Investigator or his duly assigned representative believed may affect the skin response or the interpretation of the test results.

Baseline (BL) measurements of glossiness were collected from three (3) sites on the forehead. Two (2) of the sites (one above each eye) were treated in a randomized fashion while the center site above the bridge of the nose remained non-treated to serve as a control. Two products were tested an empty vehicle cream formulation and a 2% 18B protein formulation. The vehicle control contained the following ingredients: water (>50%), caprylic/capric triglyceride (5-15%), olive oil glycereth-8 esters (1-5%), glycerin (1-5%), hydroxyethyl acrylate/sodium acryloyldimethyl taurate copolymer (1-5%), phenoxyethanol (0.1-1%), caprylyl glycol (0.1-1%), chlorphenesin (0.1-1%), tocopherol (0.1-1%), disodium EDTA (0.01-0.1%).

The treatment procedure was as follows: Approximately 32 mg of test product was applied to the designated 4 cm×4 cm test site using a micropipette. The product was spread over the test site using a clean finger cot and massaged until fully absorbed. Subjects waited in a controlled environment for approximately 30 minutes and measurements of glossiness were repeated.

The gloss of the surface was expressed by measurement of direct reflection of light sent to this surface. In the GL 200 probe head, parallel white light was sent at a 0° angle to a mirror which reflected it at a 600 angle to the skin surface. Part of the light was directly reflected at the same angle and part of the light was absorbed by the surface, scattered and reflected at different angles. The directly reflected light reflected off another mirror to a light sensor. Diffusely scattered light was also measured by a different sensor oriented above and at a 0° angle to the skin. This diffuse scattered light allowed for a diffuse scattering correction (DSC) that attempted to limit or eliminate variability due to the structure, brightness, and color of different individuals' skin.

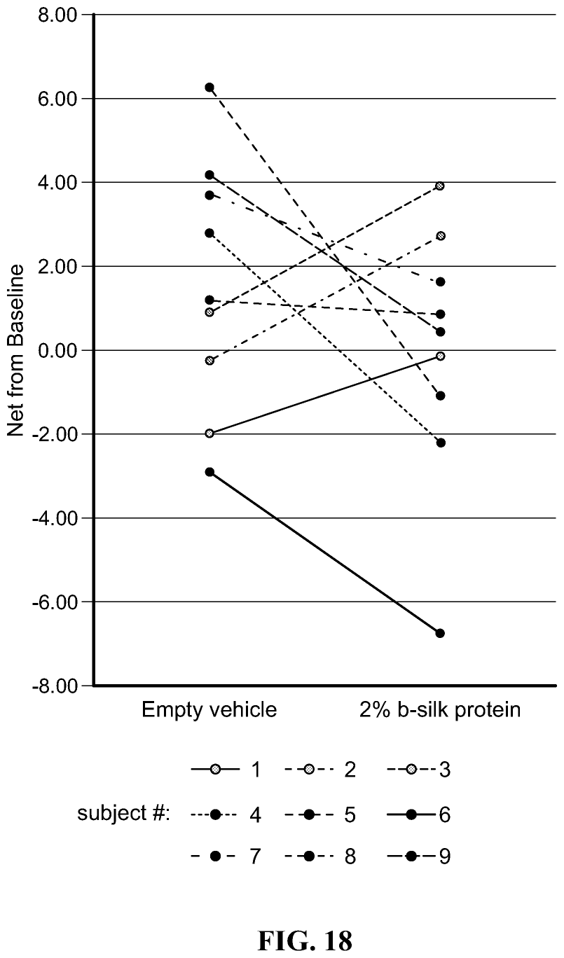

FIG. 18 shows that recombinant 18B powder has a mattifying effect on the skin when compared with an empty vehicle.

1. A method of making a silk-based composition, comprising:
mixing a recombinant silk particle and a solvent, wherein the recombinant silk particle comprises an outer shell and a hollow core, thereby forming said silk-based composition.

2. The method of claim 1, wherein said recombinant silk particle comprises an opening in said outer shell.

3. The method of claim 1, wherein the recombinant silk particle is in the form of a dry powder.

4. The method of claim 1, wherein the solvent comprises an aqueous solvent, an alcohol, an oil-based solvent, or a silicone.

5. The method of claim 1, wherein the solvent is selected from the group consisting of: water, glycerin, deionized water, olive oil, pentylene glycol, and silicone.

6. (canceled)

7. (canceled)

8. The method of claim 1, wherein the diameter of the outer shell is from 5 μm to 25 μm when the recombinant silk particle is dry and/or the diameter of the outer shell swells to up to 120 μm when mixed with the solvent.

9. (canceled)

10. The method of claim 1, wherein the outer shell thickness is less than 20% of the diameter of the recombinant silk particle.

11. (canceled)

12. The method of claim 11, wherein the recombinant silk particles are present in said composition at a concentration of from 1% to 10% wt/wt in said solvent.

13. (canceled)

14. The method of claim 1, wherein the recombinant silk particle comprises a polypeptide, the polypeptide comprising SEQ ID NO.: 2.

15.-19. (canceled)

20. The method of claim 1, further comprising spray drying a composition comprising a recombinant silk polypeptide to form a dry powder comprising said recombinant silk particle.

21. The method of claim 1, further comprising adding a dye to the silk-based composition or the recombinant silk particle.

22. The method of claim 1, further comprising adding a surfactant or humectant to the silk-based composition or the recombinant silk particle.

23.-28. (canceled)

29. The method of claim 1,
wherein the silk particle is a recombinant silk particle comprising a polypeptide comprising at least two concatenated repeat units of SEQ ID NO.: 2, and wherein the recombinant silk particle is a carrier for the solvent.

30. A method of making a silk-based solid or hydrogel, comprising:
mixing a recombinant silk particle comprising a hollow core and a solvent, wherein the recombinant silk particle functions as a carrier for the solvent, thereby forming a silk-based composition;
applying the silk-based composition to a surface; and
drying the silk-based composition to form the silk-based solid or hydrogel.

31.-33. (canceled)

34. The method of claim 30, wherein the silk-based solid or hydrogel is a bead or a film.

35.-40. (canceled)

41. The method of claim 1, further comprising mixing the silk-based composition into an emulsion to form a silk-based emulsion.

42. The method of claim 41, further comprising drying the silk-based emulsion to form a silk-based solid or hydrogel.

43.-53. (canceled)

54. A composition comprising a recombinant silk particle comprising an outer shell and a hollow core within said outer shell.

55.-100. (canceled)

101. A recombinant silk cosmetic or skincare product comprising a semi-solid, wherein the semi-solid comprises dispersed non-aggregated recombinant silk protein and a solvent.

102. (canceled)

103. (canceled)

104. A method of improving the appearance of skin comprising applying to the skin the composition of claim 54 to the skin.

105.-107. (canceled)

108. A method of cleansing a surface, comprising:
applying the composition of claim 54 to a surface to form a film or bead; and
removing the film or bead from the surface.

109.-114. (canceled)

115. A method of making a silk powder comprising recombinant silk particles, comprising:
obtaining a purified silk protein solution; and
forming a silk powder by spraying said solution with hot dry air until said silk protein solution is at or below 3% by weight moisture, wherein said silk powder comprises recombinant silk particles.

116.-127. (canceled)
